(19) United States
(12) Patent Application Publication
(10) Pub. No.: US 2024/0075122 A1
(43) Pub. Date: Mar. 7, 2024

(54) BURKHOLDERIA VACCINES AND THERAPEUTICS

(71) Applicant: University of Hawaii, Honolulu, HI (US)

(72) Inventors: Tung HOANG, Honolulu, HI (US); Ian A. MCMILLAN, Honolulu, HI (US); Yun HEACOCK-KANG, Honolulu, HI (US); Zhenxin SUN, Honolulu, HI (US); Jan ZARZYCKI-SIEK, Honolulu, HI (US); Michael H. NORRIS, Honolulu, HI (US)

(21) Appl. No.: 18/268,032

(22) PCT Filed: Dec. 30, 2021
(86) PCT No.: PCT/US2021/065668

§ 371 Date: Jun. 16, 2023

Related U.S. Application Data
(60) Provisional application No. 63/132,006, filed on Dec. 30, 2020.

(51) Int. Cl. A61K 39/02; A61P 31/04; C07K 16/12

(52) U.S. Cl. CPC A61K 39/0208; A61P 31/04; C07K 16/1203; A61K 2039/522; C07K 2317/569

(57) ABSTRACT

Virulence factors from Burkholderia species are provided by this invention as are antibodies specifically binding the same and methods of using the virulence factors and antibodies in methods of immunizing and treating or attenuating a Burkholderia infection.

### INTRODUCTION

This application claims benefit of priority to U.S. Provisional Patent Application Ser. No. 63/132,006, filed Dec. 30, 2020, the content of which is incorporated herein by reference in its entirety.

This invention was made with government support under grant nos. GM114737 and AI065359 awarded by the National Institutes of Health. The government has certain rights in this invention.

### BACKGROUND

Members of the genus Burkholderia are Gram-negative rod-shaped bacteria. Burkholderia pseudomallei (Bp) and B. mallei are primary pathogens of animals and humans with Bp being the causative agent of melioidosis, an infection for which the estimated global burden is 165,000 cases and the predicted mortality is 89,000 deaths per year. In addition to the Bp group, there is a group including 20 related bacterial species, referred to as the Burkholderia cepacia complex (Bcc), that have emerged as opportunistic pathogens capable of causing severe infections in cystic fibrosis (CF) and immunocompromised patients. Within the Bcc group, B. multivorans and B. cenocepacia most commonly cause infection in CF patients. Like melioidosis patients, patients infected with Bcc species demonstrate highly variable clinical presentations and outcomes. In some cases, patients infected with Bcc species experience a rapid decline of lung function, leading to a fatal necrotizing pneumonia. The sites of Burkholderia species infection mostly involve the lungs, bloodstream, skin, and soft tissue. The course of infection may differ depending on the bacterial strains, virulence factors, and host determinants.

While infecting a cell, Bp has a dynamic lifecycle that can be broken down into multiple stages: attachment to the host cell, host cell entry, vacuole escape, cytoplasmic replication, and protrusion towards neighboring cells culminating with the spread of infection. To establish infection in a wide range of cell types, Bp must possess a complex network of virulence factors/pathways to survive in these different environments. In this regard, there are a number of known virulence factors that have been characterized in Bp, including capsule (Cuccui et al. (2012) Infect. Immun. 80:1209-1221), lipopolysaccharide (Tuanyok et al. (2012) PLoS Negl. Trop. Dis. 6:e1453), type III and VI secretion systems (Stevens et al. (2002) Mol. Microbiol. 46:649-659; Shalom et al. (2007) Microbiology 153:2689-2699; Warawa & Woods (2005) FEMS Microbiol. Lett. 242:101-108; French et al. (2011) Proc. Natl. Acad. Sci. USA 108:12095-12100; Toesca et al. (2014) Infect. Immun. 82:1436-1444; Schwarz et al. (2014) Infect. Immun. 82:1445-1452), and BimA (Stevens et al. (2005) Mol. Microbiol. 56:40-53; Benanti et al. (2015) Cell 161:348-360). During its intracellular lifecycle, Bp attaches to host cells and is internalized by phagocytosis or an unknown mechanism (Jones et al. (1996) J. Bacteriol. 64:782-790), followed by vesicular escape using the Burkholderia secretion apparatus, a type III secretion system (T3SS_{Bsa}), to gain entry to the host cell cytoplasm (Stevens et al. (2002) Mol. Microbial. 46:649-659; Warawa & Woods (2005) FEMS Microbial. Lett. 242:101-108; French et al. (2011) Proc. Natl. Acad. Sci. USA 108:12095-12100). Bp uses BimA, which functions through molecular mimicry as an Ena/VASP analogue, to polymerize host cell actin (Benanti et al. (2015) Cell 161:348-360; Stevens et al. (2005) J. Bacteriol. 187:7857-7862) and its secondary flagella locus (French et al. (2011) Proc. Natl. Acad. Sci. USA 108:12095-12100) to move freely within the host cell cytoplasm. Spread to neighboring cells is then achieved by protruding and fusing host cell membranes with the virulence associated type VI secretion system leading to the formation of a multinucleated giant cell (MNGC)(Shalom et al. (2007) Microbiology 153:2689-2699; French et al. (2011) Proc. Natl. Acad. Sci. USA 108:12095-12100; Toesca et al. (2014) Infect. Immun. 82:1436-1444; Schwarz et al. (2014) Infect. Immun. 82:1445-1452). Although much of the Bp intracellular lifecycle has been elucidated, a number of hypothetical/putative proteins lack characterization (Winsor et al. (2008) Bioinformatics 24:2803-2804), suggesting a deficiency in the current working knowledge of Bp pathogenesis and physiology.

Bcc pathogenesis is complex in part due to genomic variations between species and strains, although several virulence factors have been identified. Bcc initiates infection through attachment to mucosal and epithelial surfaces mediated by protein and glycolipid receptors (Leitão et al. (2010) Appl. Microbiol. Biotechnol. 87:31-40; McClean & Callaghan (2009) J. Med. Microbiol. 58:1-12). There are several known Bcc factors that contribute to the attachment of epithelial surfaces including peptidoglycan associated lipoprotein (Pal)(Dennehy et al. (2016) Cell. Microbiol. 19:5), linocin (McClean et al. (2016) Infect. Immun. 84:1424-1437), OmpW (McClean et al. (2016) Infect. Immun. 84:1424-1437), a 22 kDa adhesion associated with the cable pili phenotype (Sajjan & Forstner (1993) Infect. Immun. 61:3157-3163; Sajjan, et al. (2000) J. Med. Microbiol. 49:875-885; Urban et al. (2005) Infect. Immun. 73:5426-5437), trimeric autotransporters (Mil-Homens & Fialho (2011) Front. Cell. Infect. Microbiol. 1:13; Pimenta et al. (2020) MicrobiologyOpen 9:e998), and O antigen lipopolysaccharide (LPS) (Saldias et al. (2009) J. Med. Microbiol. 58:1542-1548). LPS is also involved in inducing a strong immune response causing host cell damage and invasion by Bcc (Leitão et al. (2010) Appl. Microbiol. Biotechnol. 87:31-40; Hutchison et al. (2000) FEMS Immunol. Med. Microbiol. 27:73-77; Reddi et al. (2003) Pediat. Res. 54:297-305; Cieri et al. (2002) Infect. Immun. 70:1081-1086; Schwab et al. (2002) Infect. Immun. 70:4547-4555; Sajjan et al. (2006) Cell. Microbiol. 8:1456-1466). Bcc encodes several other virulence factors including ZmpA and ZmpB proteases that break down the extracellular matrix (Gingues et al. (2005) J. Bacteriol. 187:8247-8255; Kooi et al. (2006) Infect. Immun. 74:4083-4093), MgtC protease for macrophage survival and virulence in rat lungs (Maloney & Valvano (2006) Infect. Immun. 74:5477-5486), HtrA protease for resistance to osmotic/thermal stress (Flannagan et al. (2007) Infect. Immun. 75:1679-1689), a serine protease for liberation of iron from ferritin (Whitby et al. (2006) J. Med. Microbiol. 55:661-668), and lipase/phospholipase C for invasion and breakdown of lung surfactant (Mullen et al. (2007) Euro. J. Clin. Microbial. Infect. Dis. 26:869-877; Carvalho et al. (2007) APMIS 115:311-318). Additionally, clinical Bcc strains can be seen to have increased biofilm formation when isolated over time from the same patient and up-regulated biofilm related genes in response to quorum sensing molecules.

Given the diseases caused by Burkholderia species, there is a need for the identification of suitable targets for the generation of vaccines, therapeutics and diagnostics for these bacteria. The present invention meets this need in the art.

### SUMMARY OF THE INVENTION

Using single prokaryotic cell transcriptomics, virulence functions to several hypothetical Burkholderia proteins, described herein as Surface Attachment Proteins 1 and 2 (Sap1 and Sap2), have now been found. Accordingly, this invention provides a composition composed of (a) a Sap1 polypeptide having the amino acid sequence of SEQ ID NO:1, (b) a recombinant Sap2 polypeptide having the amino acid sequence of SEQ ID NO:10, or (c) a combination of (a) and (b), wherein the recombinant Sap1 polypeptide and/or recombinant Sap2 polypeptide is tagged and/or in admixture with an adjuvant. In some aspects, the Sap2 polypeptide further includes a signal peptide. In other aspects, the Sap1 polypeptide and/or Sap2 polypeptide composition is used in a method of immunizing against a Burkholderia infection.

This invention also provides a kit for diagnosing a Burkholderia infection which includes a recombinant Surface Attachment Protein immobilized on a substrate, wherein the Surface Attachment Protein is (a) a recombinant Sap1 polypeptide having the amino acid sequence of SEQ ID NO:1, (b) a recombinant Sap2 polypeptide having the amino acid sequence of SEQ ID NO:10, or (c) a combination of (a) and (b).

This invention further provides a composition, in particular a therapeutic composition, including a preservative and at least one Surface Attachment Protein antibody; wherein the antibody is: (a) an isolated Sap1 antibody having a variable domain including (i) a CDR1 of SEQ ID NO:14, (ii) a CDR2 of SEQ ID NO:18, and (iii) a CDR3 of SEQ ID NO:22; (b) an isolated Sap2 antibody having a variable domain including (i) a CDR1 of SEQ ID NO:26, (ii) a CDR2 of SEQ ID NO:29, and (iii) a CDR3 of SEQ ID NO:32; or (c) a combination of (a) and (b). In particular aspects, the variable domain of the Sap1 antibody includes (i) the CDR1 of SEQ ID NO:15, SEQ ID NO:16, or SEQ ID NO:17; (ii) the CDR2 of SEQ ID NO:19, SEQ ID NO:20, or SEQ ID NO:21; and (iii) the CDR3 of SEQ ID NO:23, SEQ ID NO:24, or SEQ ID NO:25. In certain aspects, the variable domain of the Sap2 antibody includes (i) the CDR1 of SEQ ID NO:27 or SEQ ID NO:28; (ii) the CDR2 of SEQ ID NO:30 or SEQ ID NO:31; and (iii) the CDR3 of SEQ ID NO:33 or SEQ ID NO:34.

A method of treating or attenuating a Burkholderia infection or Burkholderia-associated condition in a subject is also provided, which includes the step of administering to a subject in need of treatment an effective amount of a Sap1 antibody, a Sap2 antibody, or a combination thereof thereby treating or attenuating the Burkholderia infection or Burkholderia-associated condition in the subject. In one aspect, the antibody is a monoclonal antibody or nanobody. In another aspect, the Burkholderia-associated condition is melioidosis or pneumonia.

This invention further provides a live-attenuated Burkholderia pseudomallei that includes a deletion of nucleic acids encoding the BPSL0636 polypeptide of SEQ ID NO:35 and optionally the BPSS0015 polypeptide of SEQ ID NO:137, and a method of using the live-attenuated Burkholderia pseudomallei in a method of immunizing against a Burkholderia pseudomallei infection.

### BRIEF DESCRIPTION OF THE DRAWINGS

FIG. 1 shows overall infection defects of ΔBPSL0097, ΔBPSS1860, ΔBPSS1818, ΔBPSS0015 and ΔBPSL0636 mutant strains as evidenced by significant reductions in plaque diameters in HEK293T monolayers as compared to wild-type (WT) (n=3). Data represents means±s.e.m and analyzed via two-sided unpaired t-test. P values presented above relevant comparisons.

FIG. 2 shows the role for BPSL0097 and ΔPSS1860 in attachment of Bp to the surface of host cell lines RAW, HEK293T and HTB, as evidenced by reduced attachment efficiencies of ΔBPSL0097 and ΔBPSS1860 mutant strains compared to wild-type (WT) Bp (n=3). Data represents means±s.e.m and analyzed via two-sided unpaired t-test. P values presented above relevant comparisons.

FIG. 3 shows the monoclonal antibodies that specifically bind BPSL0097 (αSR1 #140) and BPSS1860 (αSR2 #1) reduce the ability of Bp to attach to HEK293T cells. The level of reduction compares to capsular polysaccharide-specific monoclonal antibody 4C4 and LPS-specific monoclonal antibody 407.

FIG. 4 shows that after vaccination and a single boost with Bp Sap1 and Sap2 subunit vaccines, mice elicited an immune response as evidenced by the production of Sap1- and Sap2-specific IgG.

FIG. 5 shows that after vaccination and a single boost with Bp Sap1 and Sap2 subunit vaccines, vaccinated mice exhibited attenuated signs of disease compared to control mice at three days post-infection, Clinical scores of 0=normal, 1=questionable illness, 2=mild but definitive illness, 3=moderate illness, 4=severe illness (moribund-euthanized), and 5=found dead. Statistical analysis carried out by unpaired t-test. ****<0.0001.

FIG. 6 shows reciprocal endpoint titers for Balb/c and C57Bl/6 mice vaccinated with a Sap1 plus Sap2 vaccine (labeled as Sap1 w/Sap2 or Sap2 w/Sap1), Sap1 only vaccine, Sap2 only vaccine, or adjuvant control (control). The limit of detection for the graph is 20 and marked by a dotted line. Statistical analysis was carried out via Mann-Whitney test and the lines represent the means with SEM. P values are as follow: ****<0.0001, ns>0.05 (not significant). All graphs represent an n=12 for each group. Sap1=BPSL0097, Sap2=BPSS1860.

FIG. 7 shows that K56-2 and LO6 Δsap1 mutant strains of Bcc exhibit a significant reduction in biofilm formation when compared to wild-type (WT) strains, and that complementation (Δsap1/comp) recovers this defect. Statistical analysis carried out by unpaired t-test and data in bar graphs represents the means±standard error of the mean (s.e.m.). P values are as follows: ***<0.001, ****<0.0001.

FIG. 8 shows that K56-2 and LO6 Δsap1 mutant strains of Bcc had a significant reduction in colony forming units (CFUs) within biofilm when compared to the wild-type strains and complemented (Δsap1/comp) strains. Statistical analysis carried out by unpaired t-test and data in bar graphs represents the means±standard error of the mean (s.e.m.). P value: *<0.05.

FIG. 9 shows the consensus sequence (SEQ ID NO:1) and high degree of amino acid sequence identity of Surface Attachment Protein 1 (Sap1) from B. mallei 6 (Bma; SEQ ID NO:2), B. pseudomallei 1026b (Bpi; SEQ ID NO:3), B. pseudomallei K96243 (BpK; SEQ ID NO:4), B. multivorans ATCC17616 (Bmu; SEQ ID NO:5), B. cenocepacia K56-2 (BcK; SEQ ID NO:6), B. cenocepacia H111 (BcH; SEQ ID NO:7), B. cenocepacia J2315 (BcJ; SEQ ID NO:8), and B. cepacia LO6 (BcL; SEQ ID NO:9). Underlined residues denote signal peptide sequences.

FIG. 10 shows the consensus sequence (SEQ ID NO:10) and high degree of amino acid sequence identity of Surface Attachment Protein 2 (Sap2) from B. pseudomallei 1026b (Bpi; SEQ ID NO:11), B. pseudomallei K96243 (BpK; SEQ ID NO:12), and B. mallei 6 (Bma; SEQ ID NO:13). Underlined residues denote signal peptide sequences.

### DETAILED DESCRIPTION OF THE INVENTION

A ‘TRANSITomic’ approach was used to profile transcriptomes of single Burkholderia pseudomallei cells as they transit through host cell infection at defined stages: vacuole entry; cytoplasmic escape and replication; and membrane protrusion, promoting cell-to-cell spread. The B. pseudomallei ‘TRANSITome’ revealed dynamic gene-expression flux during transit in host cells and identified genes that are required for pathogenesis. Several hypothetical proteins were exhibited virulence mechanisms, including attachment, cytoskeletal modulation, and autophagy evasion. Based upon these analyses, the present invention provides compositions for use in treating or attenuating Burkholderia infection.

Accordingly, the present invention provides a composition including an effective amount of an isolated, recombinant Sap1 polypeptide (also referred to herein as BPSIJ0097) or peptide thereof; an isolated, recombinant Sap2 polypeptide (also referred to herein as BPSS1860) or peptide thereof; or a combination thereof, wherein the recombinant Sap1 polypeptide and/or recombinant Sap2 polypeptide is tagged and/or in admixture with an adjuvant. In one aspect, the composition includes an isolated, recombinant Sap1 polypeptide or peptide thereof. In another aspect, the composition includes an isolated, recombinant Sap2 polypeptide or peptide thereof. In yet another aspect of the invention the composition includes both Sap1 and Sap2 polypeptide or peptides thereof.

In accordance with some aspects of the invention, suitable isolated Sap1 polypeptides or proteins include those derived from any clinically relevant species or strain of Burkholderia. The amino acid sequence of Sap1 proteins from various strains of Burkholderia that are suitable for the composition of the present invention are shown in FIG. 9 (i.e., SEQ ID Nos:2-9). SEQ ID NO:1 of FIG. 9 is a Sap1 consensus sequence demonstrating the high level of sequence identity across Sap1 proteins of various Burkholderia species. Accordingly, in one aspect of the present invention, the isolated Sap1 protein has an amino acid sequence of SEQ ID NO:1. In another aspect of the present invention, the isolated Sap1 protein has an amino acid sequence of any one of SEQ ID NOs:2-9 or a sequence having about 80-90% sequence similarity to the same, more preferably, about 95-99% sequence similarity.

The N-terminal amino acid residues of the full-length Sap1 represent the native secretion/signal sequence. Thus, the “mature” secreted form of Sap1 is represented by the 92 C-terminal amino acid residues in each of SEQ ID NOs:2-9. Correspondingly, the amino acid residues in each of SEQ ID NOs:2-9 are referred to as the “immature” form of Sap1. According to some aspects, the Sap1 polypeptide of this invention is the mature form. According to other aspects, the Sap1 polypeptide of this invention is the immature form.

In another aspect of the present invention, the composition includes an isolated immunogenic peptide of Sap1. Suitable Sap1 peptides are about 20 to about 80 amino acids in length. More preferably Sap1 peptides are between about 30-50 amino acids in length, more preferably between about 30-60 amino acids in length.

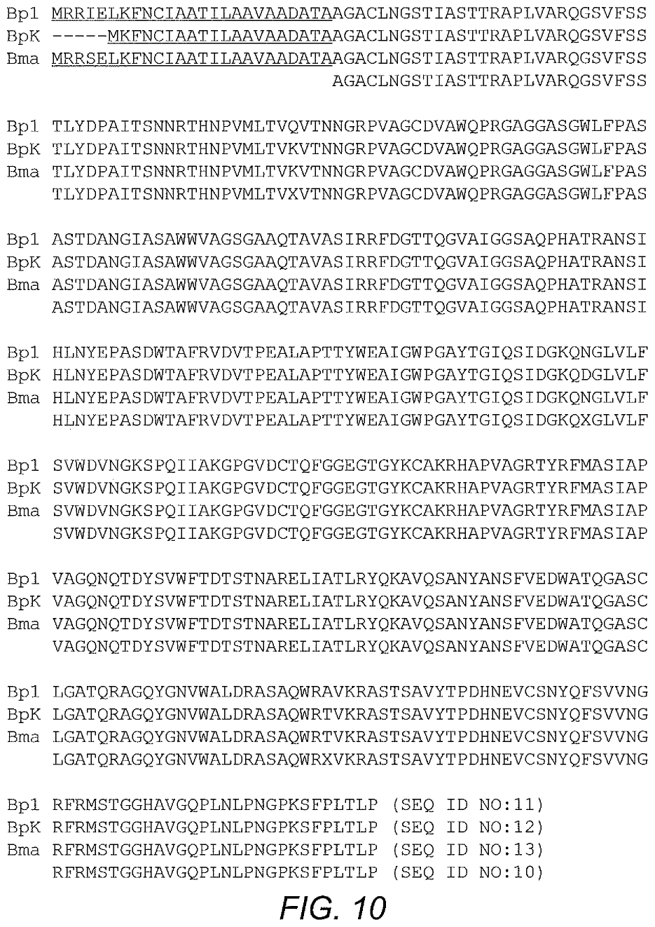

In accordance with other aspects of the invention, suitable isolated Sap2 polypeptides or proteins include those derived from any clinically relevant species or strain of Burkholderia. The amino acid sequence of Sap2 proteins from various strains of Burkholderia that are suitable for the composition of the present invention are shown in FIG. 10 (i.e., SEQ ID Nos:11-13). SEQ ID NO:10 of FIG. 10 is a Sap2 consensus sequence demonstrating the high level of sequence identity across Sap2 proteins of Burkholderia species. Accordingly, in one aspect of the present invention, the isolated Sap2 protein has an amino acid sequence of SEQ ID NO:10. In another aspect of the present invention, the isolated Sap2 protein has an amino acid sequence of any one of SEQ ID NOs:11-13 or a sequence having about 80-90% sequence similarity to the same, more preferably, about 95-99% sequence similarity.

The N-terminal amino acid residues of the full-length Sap2 represent the native secretion/signal sequence. Thus, the “mature” secreted form of Sap2 is represented by the 375 C-terminal amino acid residues in each of SEQ ID NOs:11-13. Correspondingly, the amino acid residues in each of SEQ ID NOs:11-13 are referred to as the “immature” form of Sap2. According to some aspects, the Sap2 polypeptide of this invention is the mature form. According to other aspects, the Sap2 polypeptide of this invention is the immature form.

In another aspect of the present invention, the composition includes an isolated immunogenic peptide of Sap2. Suitable Sap2 peptides are about 20 to about 350 amino acids in length. More preferably Sap2 peptides are between about 30-300 amino acids in length, more preferably between about 30-200 amino acids in length.

Unless indicated to the contrary, both the immature and the mature forms of native Sap1 and Sap2, and the sequences having less than 100% similarity with native Sap1 and Sap2 (i.e., native sequences and analogs alike, collectively referred to herein as “Sap1” and “Sap2”) may be used in the compositions and methods of the present invention.

Sap1 and Sap2 proteins and peptides of the invention may differ from the native polypeptides designated as SEQ ID NOs:2-9 and 11-13, respectively, in terms of one or more additional amino acid insertions, substitutions or deletions, e.g., one or more amino acid residues within SEQ ID NOs:2-9 and 11-13 may be substituted by another amino acid of a similar polarity, which acts as a functional equivalent, resulting in a silent alteration. That is to say, the change relative to the native sequence would not appreciably diminish the basic properties of native Sap1 or Sap2. Any such analog of Sap1 or Sap2 may be screened in accordance with the protocols disclosed herein (e.g., the cell attachment assay) to determine if it maintains native Sap1 or Sap2 activity. Substitutions within these Surface Attachment Proteins may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

Ideally, a highly purified Sap1/Sap2 preparation is used in the compositions and methods of this invention. Examples include Sap1 and Sap2 proteins or peptides purified from the species or strain of Burkholderia exemplified in FIG. 9 and FIG. 10. Methods of purifying Sap1 and Sap2 polypeptides are described herein. As used herein, an “isolated” protein or polypeptide refers to a protein or polypeptide that has been separated from other proteins, lipids, and nucleic acids with which it is naturally associated with. Purity can be measured by any appropriate standard method, for example, by column chromatography, polyacrylamide gel electrophoresis, of HPLC analysis. An isolated protein or polypeptide of the invention can be purified from a natural source, produced by recombinant DNA techniques, or by chemical methods.

For some uses of the proteins disclosed herein, it may be advantageous to use them in the form of fusion proteins. In some aspects, the Sap1 and/or Sap2 protein is fused at its N-terminus or its C-terminus to a protein, protein domain, or peptide, e.g., a tag, in particular an affinity or purification tag. Suitable tags include the peptides: WSHPQFEK (SEQ ID NO:131) available under the tradename STREP-TAG® II, EQKLISEEDL (SEQ ID NO:132) conventionally known as a myc-tag, DYKDDDDK (SEQ ID NO:133) available under the tradename FLAG®-tag, HHHHHH (SEQ ID NO:134) conventionally known as a His-tag, YPYDVPDYA (SEQ ID NO:135) conventionally known as an HA-tag, CCPGCC (SEQ ID NO:136) conventionally known as a TC-tag, or AAA conventionally known as a 3×Ala-tag; or proteins such as glutathione-S-transferase (GST), maltose binding protein (MBP), or chitin binding domain (CBD), which also allow for easy detection and/or easy purification of recombinant proteins. Further, proteins with chromogenic or fluorescent properties, such as green fluorescent protein (GFP) or yellow fluorescent protein (YFP), are also suitable tags of the present disclosure.

In another aspect of the invention, the isolated Sap1 and/or Sap2 protein or peptide thereof is linked or conjugated to an immunogenic carrier molecule. In some cases, the immunogenic carrier molecule may be covalently or non-covalently bound to the Sap1 or Sap2 protein or peptide. Exemplary immunogenic carrier molecules include, but are not limited to, bovine serum albumin, chicken egg ovalbumin, keyhole limpet hemocyanin, tetanus toxoid, diphtheria toxoid, thyroglobulin, a pneumococcal capsular polysaccharide, CRM197 (genetically detoxified form of diphtheria toxin), and a meningococcal outer membrane protein.

In aspects wherein the Sap1 and/or Sap2 composition is intended for use as a subunit vaccine, the Sap1 and/or Sap2 protein or peptide thereof is in admixture with an adjuvant. An “adjuvant,” as defined herein, is a substance that serves to enhance the immunogenicity of protein or peptide of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan.

Suitable adjuvants include, but are not limited to:
(1) aluminum salts (alum), such as aluminum hydroxide, aluminum phosphate, aluminum sulfate, etc.;
(2) calcium-based salts;
(3) silica;
(4) oil-in-water emulsion formulations (with or without other specific immunostimulating agents such as muramyl peptides or bacterial cell wall components), such as, for example,
   (a) MF59 (WO 90/14837), containing 5% squalene, 0.5% polysorbate 80, and 0.5% sorbitan trioleate (optionally containing various amounts of muramyl tripeptide phosphatidylethanolamine) formulated into submicron particles using a microfluidizer such as Model 110Y microfluidizer (Microfluidics, Newton, MA),
   (b) SAF, containing 10% squalene, 0.4% polysorbate 80, 5% pluronic-blocked polymer L121, and thr-MDP either microfluidized into a submicron emulsion or vortexed to generate a larger particle size emulsion,
   (c) RIBI™ adjuvant system (RAS), (Corixa, Hamilton, MT) containing 2% squalene, 0.2% polysorbate 80, and one or more bacterial cell wall components from the group consisting of 3-O-deacylated monophosphorylipid A (MPL™) described in U.S. Pat. No. 4,912,094, trehalose dimycolate (TDM), and cell wall skeleton (CWS), preferably MPL+CWS (DETOX™); and
   (d) a Montanide ISA;
(5) saponin adjuvants, such as those sold under the tradenames QUIL-A® or QS-21 STIMULON® (Antigenics, Framingham, MA) (see, e.g., U.S. Pat. No. 5,057,540), may be used or particles generated therefrom such as ISCOM (immunostimulating complexes formed by the combination of cholesterol, saponin, phospholipid, and amphipathic proteins) and ISCOMATRIX™ (having essentially the same structure as an ISCOM but without the protein);
(6) bacterial components (e.g., endotoxins, in particular superantigens, exotoxins and cell wall components) and lipopolysaccharides, synthetic lipid A analogs such as aminoalkyl glucosamine phosphate compounds (AGP), or derivatives or analogs thereof, which are available from Corixa, and described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxy-tetradecanoyl]-2-[(R)-3-tetradecanoyloxytetradecanoylamino]-b-D-glucopyranoside, which is also known as 529 (formerly known as RC529), which is formulated as an aqueous form or as a stable emulsion;
(7) synthetic polynucleotides such as oligonucleotides containing CpG motif(s) (U.S. Pat. No. 6,207,646);
(8) cytokines and chemokines (e.g., granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), IL-3, IL-4, IL-5, IL-6, IL-7, IL-8, IL-10, IL-12, IL-15, IL-18, interferon gamma, interferon gamma inducing factor I (IGIF), transforming growth factor beta, RANTES (regulated upon activation, normal T-cell expressed and presumably secreted), macrophage inflammatory proteins (e.g., MIP-1 alpha and MIP-1 beta), tumor necrosis factor (TNF), costimulatory molecules B7-1 and B7-2, and Leishmania elongation initiating factor (LEIF));
(9) complement, such as a trimer of complement component C3d;
(10) toll-like receptor agonists, e.g., TLR4 agonists such as glucopyranosyl lipid adjuvant (GLA);
(11) serum proteins, e.g., transferrin;
(12) viral coat proteins, e.g., rotavirus capsid VP6 protein; and
(13) block copolymer adjuvants, e.g., Hunter's TITERMAX® adjuvant (VAXCEL, Inc. Norcross, GA).

Muramyl peptides include, but are not limited to, N-acetyl-muramyl-L-threonyl-D-isoglutamine (thr-MDP), N-acetyl-normuramyl-L-alanine-2-(1′-2′dipalmitoyl-sn-glycero-3-hydroxyphosphoryloxy)-ethylamine (MTP-PE), etc.

In certain aspects, the adjuvant includes an aluminum salt. The aluminum salt adjuvant may be used to form an alum-precipitated vaccine or an alum-adsorbed vaccine. Aluminum-salt adjuvants are well-known in the art and are described, for example, in Harlow & Lane ((1988) Antibodies: A Laboratory Manual, Cold Spring Harbor Laboratory) and Nicklas ((1992) Res. Immunol. 143:489-493). The aluminum salt may include, but is not limited to, hydrated alumina, alumina hydrate, alumina trihydrate (ATH), aluminum hydrate, aluminum trihydrate, aluminum (III) hydroxide, aluminum hydroxyphosphate sulfate, Aluminum Phosphate Adjuvant (APA), amorphous alumina, trihydrated alumina, or trihydroxyaluminum.

In some aspects, the Sap1 and/or Sap2 protein compositions of the present invention are prepared by formulating Sap1 and/or Sap2 with a pharmaceutically acceptable carrier and optionally a pharmaceutically acceptable excipient. As used herein, the terms “pharmaceutically acceptable carrier” and “pharmaceutically acceptable excipient” (e.g., additives such as diluents, antioxidants, preservatives and solubilizing agents) are nontoxic to the cell or mammal being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present invention include antioxidants such as ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as polysorbate, polyethylene glycol (PEG), and poloxamers.

Sap1 and/or Sap2 protein compositions of the present invention may be prepared for storage by mixing the active ingredient(s) having the desired degree of purity with the pharmaceutically acceptable carrier and optional excipient and/or additional active agent, in the form of lyophilized formulations or aqueous solutions. The Sap1 and/or Sap2 protein compositions may be formulated as single dose vials, multi-dose vials or as pre-filled glass or plastic syringes.

Another aspect of the present invention relates to a method of immunizing against a Burkholderia infection in a subject. This method involves administering a Sap1 and/or Sap2 protein composition of the present invention, in an amount effective to immunize against Burkholderia infection in the subject. A suitable subject for treatment in accordance with this aspect of the present invention is a subject at risk of developing a Burkholderia infection. In accordance with this aspect of the invention, an effective amount of the Sap1 and/or Sap2 protein composition for administration to a subject is the amount necessary to generate a humoral (i.e., antibody-mediated) immune response. Preferably, administration of an effective amount of the composition induces a neutralizing immune response against Burkholderia in the subject, in particular a Burkholderia species that is a pathogen or opportunistic pathogen of a human or animal, e.g., B. mallei, B. pseudomallei, B. multivorans, B. cepacia or B. cenocepacia. To effectuate an effective immune response in a subject, the composition may further contain one or more additional Burkholderia antigens or an adjuvant as described supra. In an alternative aspect of the invention, the adjuvant is administered separately from the Sap1 and/or Sap2 protein composition to the subject, either before, after, or concurrent with administration of the Sap1 and/or Sap2 protein composition of the present invention. Modes of administration are known to a person skilled in the art and may be carried out parenterally, transmucosally, transdermally, intramuscularly, intravenously, intradermally, intranasally, subcutaneously, intraperitoneally, and formulated accordingly.

This invention further provides a serological assay and kit for diagnosing a Burkholderia infection and/or determining whether a subject has elicited an immune response to a Sap1 and/or Sap2 protein. Serological tests of this invention are based on monitoring or detecting the presence of anti-Sap1 and/or anti-Sap2 antibodies in a subject sample, e.g., blood, lymph, saliva, etc. In vitro methods of diagnosis/detection generally include contacting a subject sample (e.g., blood, serum, plasma, fractions of plasma, etc.) with an immobilized Sap1 and/or Sap2 protein and detecting specific binding between antibodies in the subject sample and the Sap1 and/or Sap2 proteins, either as a cocktail or as individual protein species, where the presence of specific binding is indicative of a prior, or current, infection and/or immune response to a vaccine. For example, diagnosis may use an ELISA technique or western blot, where a serum sample is diluted in PBS or other acceptable excipient, and incubated with the Sap1 and/or Sap2 proteins, where a positive result in the ELISA or a visual response in a western blot is indicative of reactivity. In a particular aspect, a conventional sandwich type assay is used. A sandwich assay is performed by first attaching the Sap1 and/or Sap2 proteins to an insoluble surface or support. The Sap1 and/or Sap2 proteins may be bound to the surface by any convenient means, depending upon the nature of the surface, either directly or through specific antibodies. The particular manner of binding is not crucial so long as it is compatible with the reagents and overall methods of the invention. They may be bound to the surfaces covalently or non-covalently, preferably non-covalently. Samples, fractions or aliquots thereof are then added to separately assayable supports (for example, separate wells of a microtiter plate) containing support-bound Sap1 and/or Sap2 proteins. Preferably, a series of standards, containing known concentrations of antibodies is assayed in parallel with the samples or aliquots thereof to serve as controls.

To facilitate the diagnosis of a Burkholderia infection and/or determine whether a subject has elicited an immune response to a Sap1 and/or Sap2 protein, the invention provides a kit. The kit of this invention includes a recombinant Surface Attachment Protein immobilized on a substrate, wherein the recombinant Surface Attachment Protein is a Sap1 polypeptide having the amino acid sequence of SEQ ID NO:1, preferably Sap1 polypeptides of SEQ ID NOs:2-9; a Sap2 polypeptide having the amino acid sequence of SEQ ID NO:10, preferably Sap2 polypeptides of SEQ ID NOs:11-13, or a combination of Sap1 and Sap2 proteins. Suitable substrates that may be used to immobilize the Sap1 and/or Sap2 polypeptides include beads, e.g., magnetic beads, membranes and microtiter plates. These are typically made of glass, plastic (e.g., polystyrene), polysaccharides, nylon or nitrocellulose. Microtiter plates are especially convenient because a large number of assays can be carried out simultaneously, using small amounts of reagents and samples. Preferably, the kit includes reagents, such as positive and negative controls; secondary antibodies for detection, where the provided secondary antibodies may be conjugated to an enzyme for detection; substrates for such enzymes; buffers and the like for performing assays. In certain aspects, the kit includes anti-SAP1 and/or anti-SAP2 antibodies (e.g., monoclonal antibodies) as positive controls.

Another aspect of the present invention relates to a composition including an effective amount of an antibody that specifically binds a Sap1 polypeptide, an antibody that specifically binds a Sap2 polypeptide, or a combination thereof, wherein the antibody is in admixture with a preservative and optionally a pharmaceutically acceptable carrier as described herein. As used herein, an “antibody” refers to an antibody, “antigen-binding fragment” of an antibody, or a genetically engineered polypeptide or glycoprotein that specifically binds an antigen to form a complex. Binding “specificity” refers to selective recognition of the antibody for a particular epitope of an antigen. The term “epitope” includes any protein determinant capable of specific binding to an immunoglobulin or T-cell receptor or otherwise interacting with a molecule. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen.

In one aspect, the antibody of the composition is an anti-Sap1 antibody or antigen-binding fragment thereof or an anti-Sap2 antibody or antigen-binding fragment thereof. In some aspects, the composition includes both Sap1 and Sap2 antibodies or antigen binding fragments thereof. Preferably, the composition includes one or more neutralizing Sap1 and/or Sap2 antibodies or antigen-binding fragments thereof. In yet another aspect, the anti-Sap1 and/or anti-Sap2 antibody composition is multivalent in that specifically binds more than one Burkholderia antigen. For example, the composition may include one or more antibodies that recognize one or more additional Burkholderia antigens, including LPS and/or CPS antigens.

For purposes of the present invention, the term “antibody” includes monoclonal antibodies, polyclonal antibodies, antibody fragments, genetically engineered forms of the antibodies, and combinations thereof. More specifically, the term “antibody” includes full length (i.e., naturally occurring or formed by normal immunoglobulin gene fragment recombinatorial processes) immunoglobulin molecules (e.g., an IgG antibody) and immunologically active fragments thereof (i.e., including the specific binding portion of the full-length immunoglobulin molecule), which again may be naturally occurring or synthetic in nature. Accordingly, the term “antibody fragment” or “antigen-binding fragment” includes a portion of an antibody such as F(ab′)₂, F(ab)₂, Fab′, Fab, Fv, scFv and engineered fragments including, e.g., domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains. Regardless of structure, an antibody fragment binds with the same antigen that is recognized by the full-length antibody, and, in the context of the present invention, specifically binds Sap1 or Sap2.

Monoclonal antibodies of the invention may be murine, human, humanized or chimeric. A humanized antibody is a recombinant protein in which the CDRs of an antibody from one species, e.g., a rodent, rabbit, dog, goat, horse, or chicken antibody (or any other suitable animal antibody), are transferred into human heavy and light variable domains. The constant domains of the antibody molecule are derived from those of a human antibody. Methods for making humanized antibodies are well known in the art. Yet other monoclonal antibodies of the present invention may be bispecific, e.g., they have specificity for both Sap1 and Sap2. Bispecific antibodies are preferably human or humanized. A suitable Sap1 antibody includes the SR1 #140 antibody disclosed herein. A suitable Sap2 antibody includes the SR2 #1 antibody disclosed herein.

The above-described antibodies can be obtained in accordance with standard techniques. For example, Sap1, Sap2, or an immunologically active fragment of Sap1 or Sap2 can be administered to a subject (e.g., a mammal such as a human or mouse). The proteins can be used by themselves as immunogens or they can be attached to a carrier protein or other objects, such as beads. After the mammal has produced antibodies, a mixture of antibody producing cells, such as splenocytes, are isolated, from which polyclonal antibodies may be obtained. Monoclonal antibodies may be produced by isolating individual antibody-producing cells from the mixture and immortalizing them by, for example, fusing them with tumor cells, such as myeloma cells. The resulting hybridomas are preserved in culture and the monoclonal antibodies are harvested from the culture medium. Methods of making and screening antibodies are well-known in the art.

In a particular aspect, the antibody of the composition is an anti-Sap1 nanobody, an anti-Sap2 nanobody, or a combination thereof. Recombinant nanobody production is based on specific nonclassic single-chain antibodies, existing naturally together with classic antibodies in Camelids (and some species of cartilaginous fishes). These specific molecules are composed of a dimer of only one short (without first constant CH1 region) heavy immunoglobulin chain and are fully functional in the absence of the light immunoglobulin chain. Only one variable domain (VHH, “nanoantibody”, “nanobody” or single-domain nanoantibody) of this antibody is necessary and sufficient for specific recognition and binding of antigen. Organization of variable domains (VHH) of nonclassic antibodies is largely similar to that of the variable domains (VH) of classic antibodies (human VH-domains of subclass IgG3 immunoglobulins have most evident homology with VH and VHH of Camelids). In both cases V-domains are composed of four conservative framework regions (FR), surrounding three hypervariable complementarity determining regions (CDRs). Methods of making and screening nanobodies are well-known in the art.

In particular aspects of this invention, the antibody composition of this invention includes an isolated Sap1 antibody having a variable domain including (i) a CDR1 with the amino acid sequence X₁RTX₂SX₃YAX₄G (SEQ ID NO:14), wherein X₁ is G or R, X₂ is I or F, X₃ is S or R, and X₄ is M or L, (ii) a CDR2 with the amino acid sequence AIX₁X₂X₃X₄X₅X₆TX₇YADSVSG (SEQ ID NO:18), wherein X₁ is N or S, X₂ is L, T or W, X₃ is S or G, X 4 is S or G, X₅ is S or G, X₆ is S, N or T, and X₇ is S, G or H, and (iii) a CDR3 with the amino acid sequence X₁X₂X₃X₄X₅W (SEQ ID NO:22), wherein X₁ is A, S or T, X₂ is D or N, X₃ is F or G, X₄ is S or A, and X₅ is S, N or Y; and/or an isolated Sap2 antibody having a variable domain including (i) a CDR1 with the amino acid sequence GSX₁FRFNAMG (SEQ ID NO:26), wherein X₁ is R or I, (ii) a CDR2 with the amino acid sequence X₁IX₂SGX₃STX₄YADSVKG (SEQ ID NO:29), wherein X₁ is S or D, X₂ is T or A, X₃ is D or G, and X₄ is V or N, and (iii) a CDR3 with the amino acid sequence QGSX₁RX₂PTX₃X₄EYX₅YW (SEQ ID NO:32), wherein X₁ is H or Y, X₂ is Y or R, X₃ is V or A, X₄ is F or Y, and X₅ is D or E.

In particular aspects, the variable domain of the Sap1 antibody includes (i) a CDR1 with the amino acid sequence GRTISSYAMG (SEQ ID NO: 15), GRTFSRYAMG (SEQ ID NO:16), or RRTFSSYALG (SEQ ID NO:17); (ii) a CDR2 with the amino acid sequence AINLSSSSTSYADSVKG (SEQ ID NO:19), AISTSGGNTGYADSVKG (SEQ ID NO:20), or AISWGGSTTHYADSVQG (SEQ ID NO:21); and (iii) a CDR3 with the amino acid sequence ADGSNW (SEQ ID NO:23), RRSVGSMSASNFASW (SEQ ID NO:24), or TNGAYW (SEQ ID NO:25). In other aspects, the variable domain of the Sap2 antibody includes (i) a CDR1 with the amino acid sequence GSRFRFNAMG (SEQ ID NO:27) or GSRFRFNAMG (SEQ ID NO:28); (ii) a CDR2 with the amino acid sequence SITSGDSTVYADSVKG (SEQ ID NO:30) or DIASGGSTNYADSVKG (SEQ ID NO:31); and (iii) a CDR3 with the amino acid sequence QGSHRYPTVFEYDYW (SEQ ID NO:33) or QGSYRRPTAYEYEYW (SEQ ID NO:34). In accordance with some aspects, the antibody is a monoclonal antibody. In accordance with other aspects, the antibody is a nanobody. Exemplary Sap1 nanobodies have amino acid sequences as set forth in SEQ ID NO:100-102. Exemplary Sap2 nanobodies have amino acid sequences as set forth in SEQ ID NO:103-104.

Ideally, Sap1 and/or Sap2 antibodies may inhibit or reduce Sap1 activity and Sap2 activity, respectively, e.g., cell attachment activity. In some embodiments, the Sap1 and/or Sap2 antibodies neutralize (e.g., substantially eliminate) Sap1 and Sap2 activity, respectively.

Another aspect of the present invention is directed to a method of treating or attenuating a Burkholderia infection in a subject. This method includes administering to a subject in need of treatment an effective amount of a Sap1 antibody (e.g., a monoclonal antibody or nanobody), a Sap2 antibody (e.g., a monoclonal antibody or nanobody), or a combination thereof, in an amount effective to treat or attenuate the Burkholderia infection in the subject. In accordance with this aspect of the invention, Burkholderia-associated conditions include, without limitation, melioidosis and pneumonia.

Administration of the antibody(ies) can be carried out by any conventional method including those described elsewhere herein. For purposes of this and other aspects of the invention, the target “subject” encompasses any animal, preferably a mammal, more preferably a human. In the context of administering a composition of the invention for purposes of treating or attenuating a Burkholderia infection in a subject, the target subject encompasses any subject that has been exposed to a pathogenic Burkholderia species (including opportunistic pathogens) and/or exhibited signs or symptoms of a Burkholderia infection. Such species of Burkholderia include, e.g., B. mallei, B. pseudomallei, B. multivorans, B. cepacia or B. cenocepacia, preferably B. pseudomallei or B. cenocepacia. Particularly susceptible subjects include infants, juveniles, adults or elderly adults exposed to a pathogenic Burkholderia species (including opportunistic pathogens), as well as immunocompromised juvenile, adults, and elderly adults, and subjects with diseases such as CF and diabetes mellitus. In the context of using compositions of the present invention to treat or attenuate a Burkholderia infection, either via active or passive vaccination, the concentration of Sap1 and Sap2 proteins or peptides or anti-Sap1 and anti-Sap2 antibodies in the composition are adequate to achieve the treatment or attenuation of Burkholderia infection, particularly the treatment or attenuation of Burkholderia in susceptible populations. In the context of using anti-Sap1 and anti-Sap2 antibody compositions to treat or attenuate a Burkholderia infection, the amounts of antibodies are capable of achieving a reduction in a number of symptoms, a decrease in the severity of at least one symptom, or a delay in the further progression of at least one symptom, or even a total alleviation of the infection.

Therapeutically effective amounts of Sap1 protein, Sap2 protein, anti-Sap1 and anti-Sap2 antibodies can be determined in accordance with standard procedures, which take numerous factors into account, including, for example, the concentrations of these active agents in the composition, the mode and frequency of administration, the severity of the Burkholderia infection to be treated (or attenuated), and subject details, such as age, weight and overall health and immune condition. General guidance can be found, for example, in the publications of the International Conference on Harmonization and in Remington's Pharmaceutical Sciences (Mack Publishing Company 1990), which is hereby incorporated by reference in its entirety. A clinician may administer Sap1 protein and/or Sap2 protein or anti-Sap1 and/or anti-Sap2 antibodies, until a dosage is reached that provides the desired or required prophylactic or therapeutic effect. The progress of this therapy can be easily monitored by conventional assays.

Therapeutically effective amounts of Sap1 protein and Sap2 protein for immunization may depend on whether adjuvant is co-administered, with higher dosages being required in the absence of adjuvant. The amount of Sap1 protein and Sap2 protein for administration sometimes varies from 1 μg to 500 μg per patient and more usually from 5 μg to 500 μg per injection for human administration. Occasionally, a higher dose of 1-2 mg per injection is used. Typically, about 10, 20, 50 or 100 μg is used for each human injection. The timing of injections can vary significantly from once a day, to once a year, to once a decade. Generally, an effective dosage can be monitored by obtaining a fluid sample from the subject, generally a blood serum sample, and determining the titer of antibody developed against the Sap1 and/or Sap2 protein or peptide, using methods well-known in the art and readily adaptable to the specific antigen to be measured. Ideally, a sample is taken prior to initial dosing and subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays.

Therapeutically effective amount of the Sap1 antibody and/or Sap2 antibody composition typically is at least 50 mg composition per kilogram of body weight (mg/kg), including at least 100 mg/kg, at least 150 mg/kg, at least 200 mg/kg, at least 250 mg/kg, at least 500 mg/kg, at least 750 mg/kg and at least 1000 mg/kg, per dose or on a daily basis. Dosages for monoclonal antibody or nanobody compositions might tend to be lower and may be at least about 5 mg/kg, at least about 10 mg/kg, at least about 15 mg/kg, at least about 20 mg/kg, or at least about 25 mg/kg. In some methods, two or more antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody in the subject. Alternatively, antibody can be administered as a sustained release formulation, in which case less frequent administration is required. Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, and preferably until the subject shows partial or complete amelioration of symptoms of disease.

The therapeutic compositions of the present invention can be administered as part of a combination therapy in conjunction with another active agent, depending upon the nature of the Burkholderia infection that is being treated. Such additional active agents include anti-infective agents, antibiotic agents, and antimicrobial agents. Representative anti-infective agents that may be useful in the present invention include vancomycin and lysostaphin. Representative antibiotic agents and antimicrobial agents that may be useful in the present invention include penicillinase-resistant penicillins, cephalosporins and carbapenems, including vancomycin, lysostaphin, penicillin G, ampicillin, oxacillin, nafcillin, cloxacillin, dicloxacillin, cephalothin, cefazolin, cephalexin, cephradine, cefamandole, cefoxitin, imipenem, meropenem, gentamycin, teicoplanin, lincomycin and clindamycin. Dosages of these antibiotics are well known in the art. The anti-inflammatory, anti-infective, antibiotic and/or antimicrobial agents may be combined prior to administration, or administered concurrently (as part of the same composition or by way of a different composition) or sequentially with the inventive therapeutic compositions of the present invention. In certain embodiments, the administering is repeated. The subject may be an infant, juvenile, adult, or elderly adult. The subject may also be an immuno-compromised juvenile, adult, or elderly adult.

Another aspect of the present invention relates to a method of identifying inhibitors of Sap1/2 activity. This method involves providing a cell population, a preparation containing Sap1 and Sap2, and a candidate Sap1/2 inhibitor. The cell population is exposed to the preparation containing Sap1 and Sap2 in the presence and absence of the candidate inhibitor, and Sap1/2-mediated cell attachment is measured in the presence and in the absence of the candidate inhibitor. The reduced level of cell attachment is indicative of an inhibitor of Sap1 and/or Sap2 activity. Inhibitors identified in such a screen are particularly useful in the prevention, treatment or attenuation of Burkholderia infection

The candidate compounds utilized in the assays described herein may be essentially any compound or composition suspected of being capable of affecting biological functions or interactions. The compound or composition may be part of a library of compounds or compositions. Alternatively, the compound or compositions may be designed specifically to interact or interfere with the biological activity of Sap1, Sap2, or Sap1 and Sap2 of the present invention.

In a further aspect, this invention provides a live-attenuated B. pseudomallei, the genome of which has a deletion of all or a portion of the nucleic acids encoding the BPSL0636 polypeptide of SEQ ID NO:35 so that a functional protein is not produced. In certain aspects, the genome of the live-attenuated B. pseudomallei has a deletion of all or a portion of the nucleic acids encoding the BPSL0636 polypeptide of SEQ ID NO:35 and a deletion of all or a portion of the nucleic acids encoding the BPSS0015 polypeptide of SEQ ID NO:137, i.e., a double knockout mutant. Deletion or knock out of the nucleic acids encoding the BPSL0636 polypeptide and optionally BPSS0015 polypeptide can be carried out as described in the Example 1 or using any other conventional method. Given the defect in invasion and complete attenuation of the BPSL0636 deletion mutant and BPSL0636/BPSS0015 double deletion mutant, these strains are particularly useful in prophylaxis. Accordingly, the invention further provides methods for immunizing against a B. pseudomallei infection by administering to a subject in need thereof a live-attenuated B. pseudomallei BPSL0636 deletion mutant or BPSL0636/BPSS0015 double deletion mutant.

The following non-limiting examples are provided to further illustrate the present invention.

### Example 1: B. pseudomallei Intracellular ‘TRANSITome’

### Materials and Methods

Bacterial Strains and Eukaryotic Cell Lines, Media, and Culturing Conditions. All manipulation of Bp was conducted in a CDC-approved and -registered BSL3 facility at the University of Hawai'i at Mānoa with all experiments approved by the Institutional Biosafety Committee and were performed using BSL3 practices following recommendations set forth in the BMBL, 5^{th} edition. Escherichia coli EPMax10B-lacI^{q}-pir⁵² was routinely used as a cloning strain. The Bp wild-type strains, K96243 and 1026b, and their derivatives were cultured in Luria Broth (LB) or 1×M9 minimal media supplemented with 20 mM glucose (MG). For selection of the glyphosate resistance gene (gat) in E. coli and Bp, MG medium supplemented with 0.3% (w/v) glyphosate was used. Murine macrophage cell line (RAW264.7, ATCC), human embryonic kidney cell line (HEK293T, ATCC), and human neuroblastoma cell line (SK-N-SH, ATCC) were used in this study for Bp infection. All cell lines were commercially available cell lines originally obtained from ATCC. The eukaryotic cell cultures were grown in Dulbecco's Modified Eagle Medium (DMEM, Hyclone) supplemented with 10% fetal bovine serum (FBS, Hyclone) at 37° C. with 5% CO₂, and the Antibiotic-Antimycotic reagent (Invitrogen) was added at 1× concentration for cell culture maintenance but omitted during Bp infection studies.

Molecular Reagents and Methods. All molecular reagents and methods of transcriptomic analysis were used as previously described (Kang et al. (2011) Genome Res. 21:925-935; Kang et al. (2015) Nat. Protoc. 10:974-984).

Macrophage Infection with Fluorescence-Tagged B. pseudomallei. The Bp wild-type strain K96243 was labeled with red- and yellow-fluorescence proteins for easy visualization during macrophage infection. Briefly, the pUC57-PS12-yfp (Norris et al. (2010) Appl. Environ. Microbiol. 76:7635-7640) was digested with NcoI, blunt-ended, and the yfp gene was ligated with the mini-Tn7-gat-rip (Norris et al. (2010) Appl. Environ. Microbial. 76:7635-7640) backbone digested with BamHI and blunt-ended. The yfp gene was in the same orientation as the rfp gene in the resulting plasmid and both genes were driven by a constitutive promoter in Bp, PC_{S12} (Barrett et al. (2008) Appl. Environ. Microbiol. 74:4498-4508). The mini-Tn7-gat-rfp-yfp plasmid was then conjugated into K96243 along with the helper plasmid pTNS3-asd_{Ec} (Kang et al. (2009) Appl. Environ. Microbiol. 75:4015-4027). Bp with insertion of the mini-Tn7 plasmid at the attTn7 site was confirmed by PCR according to known methods (Norris et al. (2009) Appl. Environ. Microbiol. 75:6062-6075). Stable expression of the RFP and YFP proteins was confirmed and used for macrophage infection studies.

The macrophage infection with Bp was carried as follows. First, the RAW 264.7 cells were seeded onto 0.17 mm PET membrane coated Membrane Slides (Carl Zeiss) that were pretreated with UV and then 150 μg/mL poly-L-lysine. The RFP-YFP-tagged Bp strain was grown to mid-log phase (OD₆₀₀˜0.8) and diluted to approximately 4×10⁵ CFU/mL in DMEM medium with 10% FBS. RAW264.7 cells were infected at a multiplicity of infection (MOI) of 0.2 for 30 minutes and extracellular Bp were washed away with 1× phosphate-buffered saline (PBS). Fresh DMEM medium supplemented with 10% FBS was then added to the membrane slides without antibiotics to ensure a valid comparison to the control. At 1, 2, 6 hours post-infection, correlating to the vacuole, cytoplasm, and protrusion stages, respectively, the membrane slides were washed with 1×PBS and immediately fixed with 1% (w/v) paraformaldehyde (PFA) for 5 minutes followed by 70% (v/v) ethanol for 30 minutes. To obtain the control Bp cells as the baseline for microarray comparison, the same diluted Bp culture as above was incubated in the DMEM medium with 10% FBS for 1 hour, harvested by centrifugation and resuspended in 1% (w/v) PFA for 5 minutes. The fixed bacteria were then smeared on to the membrane slides, treated with 70% (v/v) ethanol for 30 minutes.

To visualize Bp at different stages during macrophage infection, the macrophage plasma and vacuolar membranes were stained with FM™ 4-64FX lipophilic dye and actin was stained with Oregon Green 488 Phalloidin (Thermo Fisher Scientific). The fluorescent images were obtained on a Zeiss PALM laser catapulting system with 100× oil immersion objective. Single Bp cells from the various stages (vacuole, cytoplasmic replication, and protrusion) of infection were cut by the focused laser and catapulted with unfocused low-intensity laser into the 0.2 mL PCR tube lid containing lysis buffer (Kang et al. (2011) Genome Res. 21:925-935; Kang et al. (2015) Nat. Protoc. 10:974-984). Cells within the cytoplasm and protrusion stages carried actin tails but were differentiated by the time post-infection and visual location in respective intracellular niche. For the control condition (Bp grown in DMEM), a total of nine Bp cells were catapulted into the same PCR tube before processing. Single Bp cells and pooled control Bp cells were processed using previously described methods (Kang et al. (2011) Genome Res. 21:925-935; Kang et al. (2015) Nat. Protoc. 10:974-984).

Two-Color Microarray and Data Analysis. Transcriptomic analysis was carried out with the B. mallei/pseudomallei 70mer oligo arrays (J. Craig Venter Institute). Each Bp cell was estimated to contain less than 2 pg of total RNA (Kang et al. (2011) Genome Res. 21:925-935; Kang et al. (2015) Nat. Protoc. 10:974-984). Single Bp cells isolated directly from host were lysed, and the cDNA synthesis and amplification from single bacterium total RNA were performed (Kang et al. (2011) Genome Res. 21:925-935; Kang et al. (2015) Nat. Protoc. 10:974-984). The amplified cDNA were labeled with Cy3 or Cy5 dye and hybridized to the B. mallei/pseudomallei 70mer oligo arrays following established protocols (Kang et al. (2011) Genome Res. 21:925-935; Kang et al. (2015) Nat. Protoc. 10:974-984; Heacock-Kang et al. (2017) Mol. Microbiol. 106:976-985). Microarray slides were scanned in a GenePix 4000 microarray scanner with GenePix Pro software 5.1. Individual TIFF images from each channel were processed with Spotfinder software 3.2.1 to generate the raw data. The raw data were then normalized using MIDAS software 2.21 with low intensity filtering, LOWESS normalization, standard deviation regularization, and in-slide replicate analysis. Finally, the normalized data in technical replicates were combined, using MEV software 4.5.1, to generate gene fold-change data (P≤0.05) when comparing one single bacterium during macrophage infection to the in vitro control. The biological replicates for each infection stage were also merged using MEV software to identify the genes that were consistently up- or down-regulated through one-way ANOVA with multiple comparisons (P≤0.05). All heat maps were presented in a green-black-red color gradient; green color indicates up-regulation and red color indicates down-regulation, when the gene expression in each infection stage was compared to the control condition. Grey boxes represented the lack of expression data in a technical replicate for that gene. Reference genome of K96243 type strain was used and a total of 5797 genes were analyzed (Winsor et al. (2008) Bioinformatics 24:2803-2804).

Gene Assignment and Pathway Designation. Gene description, function prediction, and functional category assignment was assisted for some genes using Burkholderia Genome database (Winsor et al. (2008) Bioinformatics 24:2803-2804) and Kyoto Encyclopedia of Genes and Genomes (KEGG; Kanehisa & Goto (2000) Nucleic Acids Res. 28:27-30).

Real-Time RT-PCR. Validation of microarray data with Real-time RT-PCR was performed using TAQMAN® probe. Three housekeeping genes, BPSL0602, BPSL2502, and BPSS2061, that have consistent expression levels across all conditions tested were chosen based on microarray data, as well as published transcriptomic data (Ooi et al. (2013) PLoS Genet. 9:e1003795). BPSS1511, BPSL1528, and BPSL1064 showed differential expression in the TRANSITome data with up-regulation in the vacuole, cytoplasm, and protrusion, respectively, and similar expression results were observed using RT-PCR. Oligos for real-time RT-PCRs were designed using Integrated DNA Technologies Primer Quest software and are presented in Table 1.

TABLE 1

| | | SEQ ID |
|---|---|---|
| Oligo Name | Sequence (5′→3′) | NO: |
| BPSL1064-For | GCAACGCGATTTCTAC | 36 |
| BPSL1064-Probe | 56-FAM/AATTCCACGCATATCAAGGG/3BHQ_1 | 37 |
| BPSL1064-Rev | AAGGCGAACCGATAAT | 38 |
| BPSL1064-up | ACTGTTCTGGGAGCAG | 39 |
| BPSL1064-down | AGAGATCGGCGAGAAG | 40 |
| BPSL1528-For | AAAGTGCTCAATCAACG | 41 |
| BPSL1528-Probe | 56-FAM/AAACGACTATCGTTATGCGG/3BHQ_1 | 42 |
| BPSL1528-Rev | GAAGAACGTGCTGTAAC | 43 |
| BPSL1528-up | CGGCGAATTTCGACG | 44 |
| BPSL1528-down | TGATGTTCGCACGCTC | 45 |
| BPSS1511-For | CTCGAGCAACTGATCC | 46 |
| BPSS1511-Probe | 56-FAM/CTCTACGGCTTCGACGAATG/3BHQ_1 | 47 |
| BPSS1511-Rev | GTGATCGCCGAGATAG | 48 |
| BPSS1511-up | TGACGATCGAAGGCAG | 49 |
| BPSS1511-down | GCGACGAACGCATCC | 50 |
| BPSL0602-For | GAATACGCGATCAATCTG | 51 |
| BPSL0602-Probe | 56-FAM/ACTACGTGCTCGAACAGTTC/3BHQ_1 | 52 |
| BPSL0602-Rev | TCGGTGATCTCGAAAC | 53 |
| BPSL0602-up | ACATCGACCGCTGGGT | 54 |
| BPSL0602-down | GCCAGGTTCGCGATC | 55 |
| BPSL2502-For | CGTGCTGCTCTATTTG | 56 |
| BPSL2502-Probe | 56-FAM/CGACGAGCAGGATGATGAAC/3BHQ_1 | 57 |
| BPSL2502-Rev | GCGTAAACGGAATCAC | 58 |
| BPSL2502-up | GATCATGGTCGGCATC | 59 |
| BPSL2502-down | GTGCCGATCGACGAG | 60 |
| BPSS2061-For | TCAGGTCAAGCACATT | 61 |
| BPSS2061-Probe | 56-FAM/CACGACCTACTCCATCCTAA/3BHQ_1 | 62 |
| BPSS2061-Rev | CCGGACAGGTTGTTAT | 63 |
| BPSS2061-up | GCAGCAAACCATCCTC | 64 |
| BPSS2061-down | CCAAGTTCGGTCGCAG | 65 |

Each real-time PCR reaction contains 120 nM of each forward and reverse oligos, and 12 nM of probe. Real-time PCR was performed in the iCycler iQ (Bio-Rad) with the following steps: denaturation (95° C. for 10 minutes), 55 cycles of amplification and quantification (95° C. for 20 seconds and 65° C. for 45 seconds). Supermixes for all reactions were made and aliquoted into sub-supermixes for each gene assayed. Eight RT-PCR reactions were done for each gene and condition, performed for three single Bp cells as biological replicates. Data were processed and fold-changes were calculated following known methods (Peirson et al. (2003) Nucleic Acids Res. 31:e73).

Engineering of the Bp 1026b Virulence Factor Mutants. Chromosomal mutant knock-outs were attempted for 206 spatially upregulated Bp hypothetical genes in wild-type 1026b strain, using λ-RED recombineering as previously described (Kang et al. (2011) Nat. Protoc. 6:1085-1104). Mutations were generated by insertion of glyphosate resistance gene gat for selection, along with pheS gene for counter-selection for subsequent complementation (Kang et al. (2011) Nat. Protoc. 6:1085-1104). All mutants were verified using PCR with 191 of the 206 genes attempted being successfully mutated.

Engineering of the Bp 1026b Virulence Factor Complements. The 11 mutants that were determined to be defective in the intracellular replication assay (Table 4) were complemented and tested via intracellular replication assay. The complementation was done by reintroducing a single copy of the gene at their native loci in the corresponding mutant, using λ-RED recombineering as previously described and counter-selecting for the loss of pheS gene in the resulting complemented strain (Kang et al. (2011) Nat. Protoc. 6:1085-1104). Primers used in complementation are listed in Table 2. All complements were verified using PCR.

TABLE 2

| | | SEQ |
|---|---|---|
| Oligo Name | Sequence (5′→3′) | ID NO: |
| BPSL0097-up-pheS | GGATGCCCAGCAGAGCTTTG | 66 |
| | CGAATGGGAGACACGACATG | |
| BPSL0097-down-gat | GCTTGTACATCAGGATGTGG | 67 |
| | CGTTACTGCTTGATGAAGCG | |
| BPSL0636-up-pheS | GGATGCCCAGCAGAGCTTTG | 68 |
| | TGCACGAGGAAATCGAGCAG | |
| BPSL0636-down-gat | GCTTGTACATCAGGATGTGG | 69 |
| | TCGTTCAGTGGTGGTGGCCT | |
| BPSL1126-up-pheS | GGATGCCCAGCAGAGCTTTG | 70 |
| | AACGAACGTCACCCACGGTC | |
| BPSL1126-down-gat | GCTTGTACATCAGGATGTGG | 71 |
| | CGCTTCAACGCTTCGCGAAC | |
| BPSL1390-up-pheS | GGATGCCCAGCAGAGCTTTG | 72 |
| | TTCCCATCACATCGATGGCG | |
| BPSL1390-down-gat | GCTTGTACATCAGGATGTGG | 73 |
| | TATCTCACGCGCCGTTAACG | |
| BPSL1422-up-pheS | GGATGCCCAGCAGAGCTTTG | 74 |
| | TCGGCTAACTCAATGGGCAG | |
| BPSL1422-down-gat | GCTTGTACATCAGGATGTGG | 75 |
| | TCACGACGAGCAGCTCACCT | |
| BPSL2714-up-pheS | GGATGCCCAGCAGAGCTTTG | 76 |
| | CAAACGCCTAACGTTCTCCG | |
| BPSL2714-down-gat | GCTTGTACATCAGGATGTGG | 77 |
| | GTCAGGACAGCACGAGCTTC | |
| BPSS0015-up-pheS | GGATGCCCAGCAGAGCTTTG | 78 |
| | CTTTGCCGTTTCCCGTTTCG | |
| BPSS0015-down-gat | GCTTGTACATCAGGATGTGG | 79 |
| | GCCGTGCTCAAAGACGTTGC | |
| BPSS1265-up-pheS | GGATGCCCAGCAGAGCTTTG | 80 |
| | GACGGTGAGCGCCGTGGATG | |
| BPSS1265-down-gat | GCTTGTACATCAGGATGTGG | 81 |
| | TCGCCGCGCTAGCGCGATCT | |
| BPSS1780-up-pheS | GGATGCCCAGCAGAGCTTTG | 82 |
| | CCGAGACCCACGCATGCAAC | |
| BPSS1780-down-gat | GCTTGTACATCAGGATGTGG | 83 |
| | GACATTCAACGGCCGGATGC | |
| BPSS1818-up-pheS | GGATGCCCAGCAGAGCTTTG | 84 |
| | CGTTTCGGCGAGTGACCATG | |
| BPSS1818-down-gat | GCTTGTACATCAGGATGTGG | 85 |
| | ATTCGCGCTCAGCTCAGCAG | |
| BPSS1860-up-pheS | GGATGCCCAGCAGAGCTTTG | 86 |
| | GCGACGTATACCCGCGAATC | |
| BPSS1860-down-gat | GCTTGTACATCAGGATGTGG | 87 |
| | ACGCATCAGGGCAGCGTCAG | |
| BPSL0097-up-compHA | ATCCCCTGATTCCCTTTGTC | 88 |
| | AACAGCAATGGATATCAGCC | |
| | GTTGAAATTCGCCGCTATTG | |
| | CGGTC | |
| BPSL0097-dw-compHA | GATGTGCTTAAAAACTTACT | 89 |
| | CAATGGAATTAGCTCGAGTT | |
| | AGGTGTCACGCGTAGTCCGG | |
| | GACGTCGTACGGGTAGCCGC | |
| | CCTGCTTGATGAAGCGCGCGT | |
| BPSS1860-up-compHA | ATCCCCTGATTCCCTTTGTC | 90 |
| | AACAGCAATGGATATCAGCC | |
| | GTTGATTATTCGATCGAATC | |
| | CGCTTC | |
| BPSS1860-dw-compHA | GATGTGCTTAAAAACTTACT | 91 |
| | CAATGGAATTAGCTCGAGTT | |
| | AGGTGTCACGCGTAGTCCGG | |
| | GACGTCGTACGGGTAGCCGC | |
| | CGGGCAGCGTCAGCGGAAACG | |

Growth Curves of B. pseudomallei 1026b Mutants. Growth curves were initiated by growing the 1026b wild-type and various mutant strains overnight, then diluting 200× into fresh LB. The 96-well plate was incubated at 37° C. with shaking in the BioTek ELx808IU and measuring the OD₆₀₀ every 30 minutes for 30 hours. Each growth curve was done in triplicate and average was presented with the standard error of the mean (s.e.m).

Purification/Immunoblot of BPSL0097 and BPSS1860. BPSL0097 and BPSS1860 were purified using nickel affinity chromatography. BPSL0097 was purified with an N-terminal His6-tag from pViet (Hoang et al. (1999) Gene 237:361-371) in E. coli ER2566 codon plus strain under native conditions, and BPSS1860 was purified with an N-terminal His₆-tag from pViet (Hoang et al. (1999) Gene 237:361-371) in E. coli ER2566 codon plus strain under denatured conditions with 8 M urea. Purified BPSL0097 and BPSS1860 were run on a SDS-PAGE gel and blotted to a PVDF membrane. Immunoblots were carried out using the Western Breeze blocker/diluent and protocol (Thermo Fisher). A pool of melioidosis patient sera (n=7) and naïve sera (n=2) was de-identified and used to probe the blotted PVDF membrane (1:40 dilution). A secondary goat anti-human Ig-HRP antibody (1:10,000 dilution, Invitrogen) and NOVEX™ ECL Chemiluminescent Substrate Reagent Kit (Thermo Fisher) was used for detection.

Cell Infection Assays. Intracellular replication assays were carried out using a modified aminoglycoside protection assay. RAW264.7 cells were seeded at ˜80% confluence, infected at an MOI of 1 for 1 hour, washed with 1×PBS, and then fresh DMEM+FBS containing 750 μg/mL amikacin and 750 μg/mL kanamycin was added. At various time points post-infection, monolayers were washed two times with 1×PBS, lysed with 0.2% TRITON™ X-100 in PBS, and dilutions of lysates were plated onto LB to enumerate intracellular bacteria. Attachment assays were carried out with various cell lines at an MOI of 1. For attachment efficiency tests, infection was initiated similarly as the intracellular replication assay, and at 1 hour post-infection (hpi) the bacteria-containing medium was removed, the monolayers were washed 3 times with PBS, and lysed with 0.2% TRITON™ X-100 in PBS, diluted, plated onto LB to enumerate attached bacteria. The attachment efficiency was determined by dividing the attached number by the initial number of infecting bacteria. All Experiments were carried out in triplicate and error bars represent the s.e.m.

Plaque assays were carried out in confluent monolayers of HEK293T cells in 24-well plates. Bp 1026b strains were used to infect monolayers at an MOI of 1. After one hour, monolayers were washed with 1×PBS and overlaid with DMEM+FBS supplemented with 1.2% low gelling temperature agarose sold under the tradename SEAPLAQUE® (Lonza) and 750 μg/mL amikacin and 750 μg/mL kanamycin. At 24 hpi, monolayers were fixed with 4% PFA in 1×PBS for 45 minutes. Monolayers were stained with a 1% crystal violet solution for ease of viewing. Plaques were viewed with a Zeiss AxioObserver D1 and the accompanying AxioVision 64 bit 4.9.1 software was used to measure plaque diameter. Plaque assays were carried out in triplicate and 10 to 20 plaques per replicate were measured for comparison.

Chemical modulation of autophagy was carried out as previously described with minor modifications (Cullinane et al. (2008) Autophagy 4:744-753). Briefly, RAW264.7 cells were treated with either 4 μM rapamycin or 10 mM 3-methyladenine 1 hpi and bacterial cell counts determined at 24 hpi as described above.

Immunofluorescence and Transmission Electron Microscopy. The BPSL0097 and BPSS1860 mutant strains were complemented with translational HA (human influenza hemagglutinin)-tagged BPSL0097, or BPSS1860, respectively. Mutants and the complemented strains were stained with a primary antibody of mouse anti-HA conjugated to fluorescent dye sold under the tradename ALEXAFLUOR® 594 (1:200 dilution, Thermo Fisher Scientific), followed with secondary goat-anti-mouse antibody conjugated to fluorescent dye sold under the tradename ALEXAFLUOR® 488-10 nm colloidal gold conjugate (1:50 dilution, Thermo Fisher Scientific). Labeled bacterial cells were visualized using a Zeiss D1 observer fluorescence microscope and a 120 kV Hitachi HT7700 digital transmission electron microscope.

For visualization of the BPSS0015 mutant infected macrophage cells, 60-mm tissue culture dishes were treated with 150 μg/mL poly-L-lysine and RAW264.7 murine macrophages were seeded and allowed to attach overnight. Wild-type Bp 1026b and the BPSS0015 mutant strains were used to infect the monolayers as described above. At 24 hpi, the media was removed and the monolayers were fixed for 2 hours with 2.5% glutaraldehyde in 0.1 M sodium cacodylate buffer at pH 7.4. The dishes were washed twice with 0.1 M cacodylate buffer for 20 minutes each. Samples were post-fixed in 1% osmium tetroxide in 0.1 M cacodylate buffer for 1 hour then dehydrated in a graded ethanol series. Epoxy resin was used to infiltrate samples and allowed to polymerize at 60° C. for 2 days. Samples were visualized using a 120 kV Hitachi HT7700 digital transmission electron microscope. Images were captured using an AMT XR-41 2048×2048 pixel bottom-mount high-resolution camera.

For visualization of microtubule-associated protein light chain 3 (LC3) colocalization to the BPSS0015 mutant or wild-type Bp, HEK293T cells were transfected with pEGFP-LC3 (Addgene) using transfection reagent sold under the tradename LIPOFECTAMINE® 2000 (Invitrogen) following the manufacturers protocol. Wild-type Bp 1026b and BPSS0015 mutant strains were used to infect, as described above, the monolayers stably expressing LC3-GFP. At 24 hpi, monolayers were fixed with 1% PFA for 1 hour, permeabilized with 0.2% TRITON™ X-100, stained with DAPI and FM™ 4-64FX lipophilic dyes, and visualized using Zeiss D1 observer fluorescence microscope.

For visualization of BPSS1818 mutant-infected macrophage cells, the infected monolayer was stained by red plasma membrane stain, or DAPI and β-tubulin antibody conjugated with a fluorescent dye sold under the tradename ALEXAFLUOR® 594 (1:50 dilution, Cell Signaling Technology). Images were captured with an AxioObserer D1 and accompanying Axiovision 4.9.1 software. Multi-color fluorescent images were captured with the multichannel fluorescence acquisition module of the Axiovision software. Images were deconvolved using the image) plugin Iterative Deconvolve 3D.

Live-Cell Time-Lapse Imaging. Light microscopy of infected cell monolayers was carried out as described (Norris et al. (2010) Appl. Environ. Microbiol. 76:7635-7640), except for a few modifications. Glass bottom 12-well plates were obtained from MatTek Corporation and treated with 150 μg/mL poly-L-lysine. Monolayers were seeded and infected with bacteria in 200 μL of DMEM+FBS at an MOI of 10. After 1 hour, the medium containing bacteria was removed, the monolayers were washed two times with 1×PBS, and DMEM containing 750 μg/mL amikacin and 750 μg/mL kanamycin was added for the remainder of the experiment. Live-cell imaging was taken on an Olympus microscope equipped with the Weather Station incubation system at 37° C. with 5% CO₂. Images were captured at 1 frame/5 minutes for 24 hours. Images were compiled into videos using ImageJ.

Animal Studies. BALB/c mice between 4 and 6 weeks of age were purchased from Charles River Laboratory. Animals were anesthetized with 100 mg/kg ketamine plus 10 mg/kg xylazine and infected with Bp via the intranasal (i.n.) route (Propst et al. (2010) Infect. Immun. 78:3136-3143). Groups of mice (n=5) were challenged with a dose of 4,500 CFU (5× LD₅₀ for wild-type Bp 1026b), monitored daily for disease symptoms, and euthanized according to pre-determined humane endpoints. The lungs, liver, and spleen of surviving mice were removed, homogenized, and serial diluted to determine bacterial burdens. Statistical differences in survival times were determined by Kaplan-Meier curves followed by the log-rank test.

Ethics. The animal studies described in this manuscript were conducted in compliance with the NIH (National Institutes of Health) Guide for the Care and Use of Laboratory Animals and approved by the Institutional Animal Care and Use Committee at the University of Hawai'i at Mānoa. Melioidosis patient sera samples used in immunoblot analysis were de-identified. Blood samples were collected following informed consent under ethics approval provided by Townsville Hospital and Health Services, Australia.

Statistics and Reproducibility. Microarray data were analyzed in MEV software 4.5.1. All other statistical analyses were completed in Prism software 6.0. All experiments were done in triplicate unless otherwise noted, and all the data points were graphed for quantitative analyses. All qualitative microscopy experiments were done in triplicate. All experiments were repeated independently at least once, and replications were successful.

### Results and Discussion

Bp Gene Expression Flux in Host Cells. Bp transiting through the host experiences various environmental niches, starting from host cell entry into an intracellular vacuole, escaping from the vacuole into the host cell cytoplasm, and finally protruding toward neighboring host cells, spreading the infection (French et al. (2011) Proc. Natl. Acad. Sci. USA 108:12095-12100). Therefore, it was hypothesized that, as Bp transits through its intracellular lifecycle, gene-expression is altered to accommodate each unique environmental niche. To probe this hypothesis, laser capture microdissection (LCM; Emmert-Buck et al. (1996) Science 274:998-1001) and total transcript amplification (Kang et al. (2015) Nat. Protoc. 10:974-984) were used to isolate single Bp cells at each stage of intracellular infection and determine their transcriptional profiles, hereafter referred to as the Bp TRANSITome. Comparing single Bp cells isolated from various stages of intracellular infection to those grown in vitro (via microarray analysis, it was observed that 1,953 genes were differentially expressed in a stage-specific manner. Many genes showed niche-specific expression, indicating dynamic global control of functions at each stage of infection. Biological triplicates from each stage of infection showed high reproducibility for many genes, supporting the validity of this approach to analyze the gene expression of intracellular pathogens. Genes showing high reproducibility likely represent conserved functions while genes showing high variability between biological triplicates could be distinguished as a class of genes to study due to variable expression during intracellular infection. Microarray data was validated via qRT-PCR on three selected stage specific genes and three housekeeping genes, exhibiting high correlation to validate this method for the investigation of intracellular pathogens.

Known virulence factors showed specific expression patterns during the three defined stages of Bp intracellular infection. For example, Bp uses its T3SS_{Bsa} to escape the vacuole to gain entry into the host cell cytoplasm (Gong et al. (2011) PLoS One 6:e17852) and this was confirmed by the up-regulation of numerous type III secretion system genes during the vacuole stage of infection (e.g., BPSS1539, BPSS1401, BPSS1620, BPSS1551, BPSS1544, BPSS1535, BPSS1616, BPSS1592, and BPSS1618). All type VI secretion systems present in the Bp genome (i.e., T6SS-1, T6SS-2, T6SS-3, T6SS-4, T6SS-5, and T6SS-6) were differentially expressed compared to Bp grown in vitro, indicating that they are important for maintenance of intracellular infection beyond the known function of host cell membrane fusion (Warawa & Woods (2005) FEMS Microbiol. Lett. 242:101-108; Toesca et al. (2014) Infect. Immun. 82:1436-1444; Schwarz et al. (2014) Infect. Immun. 82:1445-1452). While only portions of each type VI secretion system showed expression in the TRANSITome, including a lack of Hcp1 (BPSS1498) expression, it was posited that this was due to the limited temporal resolution. Other significant pathways including flagella and chemotaxis genes (BPSL0269, BPSL0275, BPSL0281, BPSL3309, BPSL3319, BPSL0860, BPSL0268, BPSL3299, BPSL3300, BPSL3302, BPSL3303, BPSL3305, BPSL3306, and BPSL3307), pilus and fimbriae genes (BPSS2187, BPSS2189, BPSS2190, BPSS0092, BPSS1895, BPSS0091, BPSS0120, BPSS1893, BPSS1899, BPSS1599, BPSS1600, BPSS1891, BPSS2028, BPSS3173, BPSS2193, BPSS1897, BPSS1593, and BPSS2198), aerobic metabolism genes (BPSL1220, BPSL1223, BPSL1214, BPSL1606, BPSL1221, BPSL1219, and BPSL1222) and anaerobic energy metabolism genes (BPSL2307, BPSL1604, BPSL2311, BPSL2310, BPSL2312, BPSL2313, and BPSS1158), phosphate transport genes (BPSL1360, BPSL1361, BPSL1362, BPSL1363, BPSL1364, and BPSS1566), and potential virulence factors (BPSS0769, BPSL1431, BPSS0746, BPSL1064, BPSL1176, BPSS2052, BPSL1903, BPSL1375, BPSL2264, BPSL0393, BPSL1411, BPSL1637, BPSL0803, BPSS1795, BPSS2341, BPSS1740, BPSS1728, and BPSS1741) showed significant differential regulation throughout the Bp TRANSITome highlighting their potential importance during infection. Notably there were many genes of unknown functions were differentially regulated throughout the Bp TRANSITome.

TRANSITome Reveals Uncharacterized Virulence Factors. The Bp TRANSITome is composed of approximately 30% of genes annotated as hypothetical or putative, having no assigned function. These genes were investigated for their contribution to Bp intracellular infection. Specifically, 206 genes were targeted for knockout and 191 in-frame deletional mutants of were successfully created for the genes showing distinct expression patterns during the Bp intracellular lifecycle. To assess their role in intracellular infection, these hypothetical/putative genes were deleted in the naturally competent prototype strain Bp 1026b using λ-red recombineering (Kang et al. (2011) Nat. Protoc. 6:1085-1104). Of the hypothetical genes that showed stage-specific expression, approximately 100 genes showed high expression in the vesicular stage of infection, while approximately 55 genes showed up-regulation during cytoplasmic replication, and approximately 74 genes were highly expressed during the process of spreading by protrusion towards neighboring cells. The stage-specific expression of these hypothetical proteins indicates that they contribute to different processes during intracellular infection.

The 191 mutants were screened with a qualitative cell fusion assay in RAW264.7 murine macrophages, identifying 11 mutants that showed reduced cell fusion compared to wild-type Bp. Seven of the attenuated mutants were in genes up-regulated during the vacuole stage of infection (i.e., BPSL0097, BPSL0636, BPSL1126, BPSL1390, BPSL2714, BPSS1265, and BPSS1780), two mutants in genes highly expressed in the cytoplasm (i.e., BPSL1422 and BPSS1818), and two highly expressed during the protrusion stage of infection (i.e., BPSS0015 and BPSS1860). Of these 11 genes, four, i.e., BPSL0097, BPSL0636, BPSL1126, and BPSL1422, have not been identified in the literature while seven have had some reference. BpeT and BpeS, regulators of the RND efflux pump BpeEF-OprC, responsible for co-trimoxizole resistance, also control expression of BPSL1390, BPSL2714 and BPSS1780 are co-expressed with 165 other genes under similar conditions. In addition, a homolog of BPSS1780 was detected in purified outer membrane fractions from B. mallei (Schell, et al. (2011) J. Proteome Res. 10:2417-2424). BPSS0015 is expressed in early stationary phase culture of Bp, but further characterization of the function of this protein has not been carried out (Wongtrakoongate, et al. (2011) J. Biomed. Biotechnol. 2011:530926-530926). BPSS1265 was one of forty-nine genes deleted during clinical treatment of melioidosis but no characterization of the function of this gene was undertaken (Chantratita et al. (2011) Proc. Natl. Acad. Sci. USA 108:17165-17170). BPSS1818 was down-regulated in a N-acylhomoserine lactone synthase mutant strain of Bp indicating that it is potentially tied to quorum sensing regulation (Ooi et al. (2013) PLoS Genet. 9:e1003795). BPSS1860 has been previously identified as part of the Bp core secretome suggesting an extracellular function (Vander Broek et al. (2015) Mol. Cell Proteomics 14:905-916). All references to these genes were peripheral suggesting a more in-depth study should be undertaken. Therefore, a detailed functional characterization of all 11 genes identified through the Bp TRANSITome was undertaken.

The involvement of these hypothetical proteins during the course of host cell infection was assessed via a quantitative attachment and invasion (Table 3), and intracellular replication (Table 4) assays.

TABLE 3

| | | |
|---|---|---|
| | Attachment Frequency¹ | Invasion frequency¹ |
| Gene ID | (P value)² | (P value)² |

| | | | | |
|---|---|---|---|---|
| BPSL0097 | 5 ± 3 | (0.0070) | 55 ± 10 | (0.1145) |
| BPSL0636 | 60 ± 18 | (0.2046) | 25 ± 10 | (0.0285) |
| BPSL1126 | 41 ± 8 | (0.0432) | 90 ± 15 | (0.7096) |
| BPSL1390 | 29 ± 5 | (0.0204) | 285 ± 100 | (0.1435) |
| BPSL1422 | 33 ± 6 | (0.0260) | 40 ± 10 | (0.0550) |
| BPSL2714 | 44 ± 6 | (0.0431) | 70 ± 5 | (0.2193) |
| BPSS0015 | 52 ± 22 | (0.1740) | 45 ± 9 | (0.0651) |
| BPSS1265 | 122 ± 3 | (0.3113) | 150 ± 9 | (0.0835) |
| BPSS1780 | 35 ± 10 | (0.0372) | 100 ± 36 | (1.000) |
| BPSS1818 | 46 ± 4 | (0.0451) | 165 ± 48 | (0.2810) |
| BPSS1860 | 10 ± 2 | (0.0084) | 110 ± 20 | (0.7415) |

¹Values represent mean percent of wildtype Bp 1026b attachment, invasion, or intracellular replication at specified times plus or minus the standard error mean.
²Unpaired t test (two-sided) comparing each mutant to wildtype Bp 1026b. Significant values are bolded.

TABLE 4

Mutant intracellular replication¹ (P value)²

| Gene ID | 2 hpi | 4 hpi | 16 hpi | 24 hpi |
|---|---|---|---|---|
| BPSL0097 | 17 ± 12 | 50 ± 25 | 97 ± 25 | 16 ± 1 |
| | (0.0196) | (0.2302) | (0.9264) | (<0.0001) |
| BPSL0636 | 94 ± 8 | 175 ± 66 | 101 ± 12 | 71 ± 28 |
| | (0.7891) | (0.3486) | (0.9171) | (0.3739) |
| BPSL1126 | 37 ± 5 | 0 ± 0 | 93 ± 9 | 108 ± 6 |
| | (0.0290) | (0.0161) | (0,6915) | (0.3340) |
| BPSL1390 | 71 ± 34 | 75 ± 43 | 107 ± 1 | 100 ± 5 |
| | (0.4970) | (0.6433) | (0.5136) | (0.9027) |
| BPSL1422 | 25 ± 2 | 25 ± 25 | 69 ± 14 | 95 ± 7 |
| | (0.0153) | (0.1012) | (0.1586) | (0.6401) |
| BPSL2714 | 94 ± 25 | 25 ± 25 | 75 ± 13 | 18 ± 0 |
| | (0.8615) | (0.1012) | (0.2182) | (<0.0001) |
| BPSS0015 | 44 ± 15 | 625 ± 195 | 138 ± 10 | 15 ± 1 |
| | (0.0783) | (0.0560) | (0.0628) | (<0.0001) |
| BPSS1265 | 144 ± 27 | 200 ± 66 | 115 ± 6 | 21 ± 2 |
| | (0.2434) | (0.2302) | (0.3048) | (0.0001) |
| BPSS1780 | 196 ± 25 | 275 ± 50 | 132 ± 34 | 17 ± 1 |
| | (0.0366) | (0.0352) | (0.4235) | (<0.0001) |
| BPSS1818 | 85 ± 14 | 150 ± 87 | 114 ± 16 | 16 ± 1 |
| | (0.5401) | (0.6087) | (0.4997) | (<0.0001) |
| BPSS1860 | 79 ± 8 | 200 ± 25 | 82 ± 13 | 23 ± 2 |
| | (0.3475) | (0.0474) | (0.3657) | (0.0001) |

¹Values represent mean percent of wildtype Bp 1026b attachment, invasion, or intracellular replication at specified times plus or minus the standard error mean.
²Unpaired t test (two-sided) comparing each mutant to wildtype Bp 1026b. Significant values are bolded.

The BPSL0097 and BPSS1860 mutants showed drastic decreases in attachment at five and ten percent of wild-type, respectively, while other mutants showed moderate defects in this process (Table 3). The BPSL0636 mutant was the only strain to show a significant defect in invasion with a 75 percent decrease compared to wild-type (Table 3). Six mutants, BPSL0097, BPSL1126, BPSL1390, BPSL1422, BPSL2714, and BPSS0015, showed defects in intracellular replication between 2 and 6 hours post-infection, ranging from 17-75 percent wild-type replication while eight mutants, BPSL0097, BPSL0636, BPSL2714, BPSS0015, BPSS1265, BPSS1780, BPSS1818, and BPSS1860, showed 15-71 percent wild-type replication at 24 hours post-infection (Table 4). Significant defects were observed at 24 hours post-infection for the BPSL0097, BPSL2714, BPSS0015, BPSS1265, BPSS1780, BPSS1818, and BPSS1860 mutants (Table 4). All 11 hypothetical genes have hundreds of orthologs in Burkholderia strains signifying that they have conserved functions. In vitro growth and complementation analyses showed that the defects during intracellular replication were not caused by reduced in vitro fitness or polar effect of these mutations, which validates these genes as virulence factors.

To further evaluate the roles of these genes in Bp pathogenesis, multiple established infection models were employed including HEK293T plaque formation, live cell imaging of RAW264.7 cell infection, and acute melioidosis infection in BALB/c mice (French et al. (2011) Proc. Natl. Acad. Sci. USA 108:12095-12100; Propst et al. (2010) Infect. Immun. 78:3136-3143). Five of the mutants, BPSL1126, BPSL1390, BPSL1422, BPSL2714, and BPSS1780, showed disease progression comparable to wild-type Bp, indicating that even though they showed attenuation in the cell infection models, these genes were not required for pathogenesis in vivo. The BPSS1265 mutant showed delayed morbidity with 60% survival of mice over the duration of the study, indicating that this gene could be important for acute infection but attenuation does not extend to chronic forms of murine melioidosis. The BPSL0636 mutant showed significant attenuation in cell culture, marked by a significant reduction in plaque diameter during infection of HEK293T cells (FIG. 1). In RAW264.7 cells, it was observed that the BPSL0636 mutant replicated to wild-type Bp levels during the early stages of infection, but a major delay was detected in the spread and/or fusion to host cells during late stages of infection (Tables 3 and 4). In addition to attenuation in two drastically different cell models, mice infected with the BPSL0636 mutant survived the entire length of the in vivo study, highlighting the importance of this gene in Bp pathogenesis. Eighty percent of surviving mice completely cleared the BPSL0636 mutant, while a single mouse had residual infection in the lungs. This indicated that the BPSL0636 mutant could serve as a base strain for potential live-attenuated vaccines against melioidosis. Beyond the defect in invasion and complete attenuation of the BPSL0636 mutant in BALB/c mice, there was no obvious indication as to the pathogenic mechanism of this gene during infection. As described below, several other mutants, BPSL0097, BPSS1860, BPSS1818, and BPSS0015, showed similar levels of attenuation in multiple cell lines and BALB/c mice, and additional experiments alluded to their possible pathogenic mechanisms during infection.

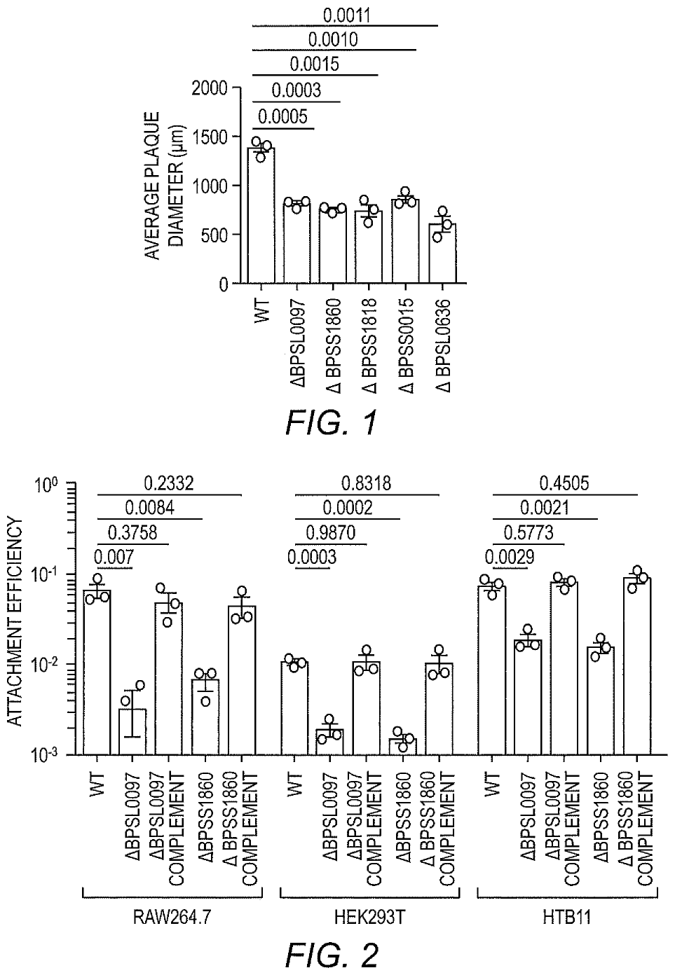

Discovery of Two Attachment Proteins. Mutants of BPSL0097 and BPSS1860 showed an attenuated phenotype when tested in HEK293T cells (FIG. 1, indicated by reduced plaque numbers and sizes) and the BALB/c model of melioidosis (100% and 80% survival, respectively). These genes were upregulated in the initial stage of cell infection and constitutively expressed throughout the Bp TRANSITome, respectively. As attachment to host cells occurs at the initial stages of infection before internalization, it was determined whether these predicted outer membrane proteins have a role during this process. Analysis of the predicted coding regions of BPSL0097 and BPSS1860 indicated that each has signal sequences indicating that they are secreted proteins and likely presented on the cell surface (Almagro Armenteros et al. (2019) Nat. Biotechnol. 37:420-423). In conjunction with this observation, mutants of BPSL0097 and BPSS1860 exhibited a significant reduction in attachment efficiency in three cell lines, RAW264.7, HEK293T, and HTB11 cells, strongly indicating an attachment function associated with these two genes (FIG. 2). A double mutant attaches at four percent of wild-type Bp, a further reduction from the five or ten percent of single BPSL0097 or BPSS1860 mutants (P=0.0512, P-0.0101, respectively, comparing the double to single mutants) indicating that other Bp attachment factors are present. Although the loss of both BPSL0097 and BPSS1860 did not result in a complete abolishment of Bp attachment to host cells, the additional reduction in attachment efficiency in the double mutant indicates that these adhesins operate separately. To validate that these proteins are involved in attachment, wild-type Bp was compared to each mutant via live-cell imaging. Wild-type Bp moved through the extracellular milieu and remained in contact for an extended period of time when encountering host cells. In contrast, BPSL0097 or BPSS1860 mutants came into contact with host cells for a limited period of time, further validating these proteins as host cell attachment factors. While these observations indicate that both proteins are essential for the initial stages of attachment, the role of BPSS1860 during the later stages has not been determined as it is expressed throughout the TRANSITome.

To confirm the prediction that BPSL0097 and BPSS1860 are presented on the outer surface of Bp, complemented strains expressing hemagglutinin (HA) tagged fusion proteins (BPSL0097::BPSL0097-HA or BPSS1860::BPSS1860-HA) were generated. The HA-tagged fusion strains were stained positively, via immunofluorescence (IF) with an anti-HA antibody, confirming that these proteins are located on the surface of Bp. Because BPSL0097-HA appeared to be located on the periphery of the bacterium and exhibited higher fluorescence signals on the poles, immunogold labeling (IG) and transmission electron microscopy (TEM) was conducted to gain a better resolution of each protein's distribution on the bacterial surface. BPSL0097-HA showed localization limited to the poles of Bp, largely agreeing with the IF result. On the contrary, BPSS1860-HA showed an even distribution across the bacterial surface via IF and this was validated by IG TEM. Since these two surface proteins are required for full pathogenesis in vivo, their immunogenicity was further evaluated in clinical melioidosis via immunoblot against serum from patients with melioidosis. While BPSL0097 showed no reaction to patient sera, purified BPSS1860 was recognized specifically by pooled patient sera demonstrating its use as a diagnostic target. The lack of immunogenicity to BPSL0097 was likely due to low presentation on the bacterial cell surface, lack of immunogenic protein sequence, or a representation of the number of sera samples pooled (n=7). Taken together, the data presented here highlights the discovery of these attachment proteins, BPSL0097 and BPSS1860, as virulence factors independently required for the progression of Bp infection.

BPSS1818 Modulates Host Cell Tubulin. Highly expressed in the cytoplasm, a mutant of BPSS1818 also showed changes in phenotype during in vitro and in vivo infections. Specifically, fused RAW264.7 cells infected with the BPSS1818 mutant showed extended host cell cytoskeleton and distended MNGCs compared to wild-type Bp. In addition, RAW264.7 cells infected with the BPSS1818 mutant showed elongated microtubules with pronounced fibers when magnified. In vitro growth and complementation analyses showed that these defects were not caused by reduced in vitro fitness, secondary mutation, or polar effect, indicating that mutation of BPSS1818 was responsible for the observed phenotypes. The mutant of BPSS1818 was highly attenuated in RAW264.7 cells (Tables 3 and 4) as well as in HEK293T cells (FIG. 1). More importantly, the BPSS1818 mutant was 100% attenuated in BALB/c mice, indicating that it is essential for in vivo pathogenesis. To better investigate the associated pathogenic function of this gene, in vitro infection models were used. During infection of RAW264.7 cells with the BPSS1818 mutant, a major phenotypic change in the overall monolayer morphology was observed when compared to cells infected with wild-type Bp. Cells infected with the BPSS1818 mutant appeared to be varied in overall cytoskeletal structure after extensive host cell fusion, and the ‘stretched-out’ MNGCs were unable to collapse into a spherical structure. This finding indicated that BPSS1818 modulates the host cell cytoskeleton leading to this phenotype in the absence of BPSS1818. Modulation of the host cell cytoskeleton components myosin, actin, and tubulin by BPSS1818 was determined by IF. There were no morphological changes in myosin and actin filaments, between RAW264.7 macrophage cells infected with the BPSS1818 mutants and wild-type Bp. On the other hand, RAW264.7 cells infected with the BPSS1818 mutant showed variations in the morphology of tubulin, as noted by elongated polymers when compared to the wild-type Bp infected host cells. BPSS1818 is a predicted inner membrane protein indicating that the modulation of tubulin is indirect and possibly requires other components. Overall, the data support the contention that BPSS1818, which indirectly modulates host cell tubulin, is required for full pathogenesis of Bp. Bp has been shown previously to modulate host cell actin (Benanti et al. (2015) Cell 161:348-360), but this appears to be the first report showing that Bp affects tubulin during infection of the host cell.

Bp Evades Autophagy Clearance via BPSS0015. The Bp TRANSITome revealed that the BPSS0015 gene was highly expressed in the protrusion stage during host cell infection, indicating its importance for later steps of the infection lifecycle. In vitro growth and complementation analyses showed that these defects were not caused by reduced in vitro fitness, secondary mutation, or polar effect, indicating that mutation in BPSS0015 was responsible for the observed phenotypes. A mutant of this gene showed a significant decrease in intracellular replication and plaque sizes in RAW264.7 and HEK293T cells (FIG. 1), respectively. When used to infect BALB/c mice at a lethal dose, all mice survived during the entire study period, indicating that the BPSS0015 gene is an essential virulence determinant for Bp pathogenesis in vivo. Unlike mutants of BPSS1818, BPSL0636, and BPSS1860, the BPSS0015 mutant was able to persist and disseminate to the spleen and liver of surviving mice. Upon closer examination using the cell infection model, the BPSS0015 mutant appeared to be trapped in membrane-bound structures. To confirm this observation, RAW264.7 cells infected with wild-type Bp and the BPSS0015 mutant were processed for TEM. The BPSS0015 mutant was encompassed by single and double membrane-bound vacuoles, properties of autophagy clearance, while wild-type Bp are not associated with any membrane-bound structures within the cytoplasm. A common marker of autophagy, LC3 (Kabeya et al. (2000) EMBO J. 19:5720-5728), was then chosen to determine whether BPSS0015 was linked to autophagy evasion. HEK293T cells stably expressing LC3-GFP were infected with the BPSS0015 mutant and wild-type Bp to assess variations in co-localization with the host cell LC3. While wild-type Bp did not associate with the LC3-GFP puncta, the BPSS0015 mutant showed co-localization. Although a previous report showed that wild-type Bp does associate with LC3 in RAW264.7 macrophages at very low levels during the first six hours of infection (Cullinane et al. (2008) Autophagy 4:744-753), that association was not observed in HEK293T cells at 24 hours post-infection. In addition, type three secretion system effectors, BopA and BipD, were previously identified in the reduction of LC3-associated phagocytosis during early stages of infection (Gong et al. (2011) PLoS One 6:e17852), indicating that Bp possesses multiple mechanisms to avoid intracellular clearance during various stages of host cell transit. These data taken together indicate that the BPSS0015 mutant is unable to avoid host cell autophagy clearance during the late stages of infection. To demonstrate this, rapamycin and 3-methyladenine were used to control the level of host cell autophagy (Cullinane et al. (2008) Autophagy 4:744-753). Rapamycin, a stimulator of host cell autophagy, reduced the intracellular burden of wild-type Bp and the BPSS0015 mutant compared to the control infection. An infection supplemented with 3-methyladenine, a suppressor of host cell autophagy, showed no effect on wild-type Bp intracellular replication, further supporting the ability of wild-type Bp to avoid host cell autophagy clearance. On the contrary, the BPSS0015 mutant aided by 3-methyladenine was able to recover its ability to replicate within the host cells. Taken together, the data indicate that Bp is able to avoid host cell autophagy during the late stages of infection and that the BPSS0015 gene is involved in this autophagy evasion mechanism. Although previous studies have shown that during initial stages of infection (<6 hours post-infection) Bp avoids LC3-associated phagocytosis (Gong et al. (2011) PLoS One 6:e17852; Cullinane et al. (2008) Autophagy 4:744-753), this is the first investigation to identify a gene involved in Bp evasion of autophagy during late stages of infection.

Model of Bp Intracellular Pathogenesis. The intracellular TRANSITome of Bp was investigated. Thousands of known and unknown genes and pathways undergo dynamic gene-expression flux as Bp transits through distinct environmental niches in the host cells. The Bp TRANSITome led to the discovery of several virulence factors that are required for complete Bp pathogenesis. Comprehensive screens of 191 mutants and functional characterizations determined potential functions for some of these virulence factors during Bp infection of host cells. Based on the data presented, functions to four of these virulence factors were assigned: BPSL0097 and BPSS1860 are surface attachment proteins, BPSS1818 is a modulator of host cell tubulin, and BPSS0015 is a factor involved in evasion of host cell autophagy. These virulence determinants have the potential to be exploited as therapeutic targets and vaccines against melioidosis.

### Example 2: Subunit Vaccines, Monoclonal Antibodies and Nanobodies

As described herein, two virulence factors, BPSL0097 (also referred to herein as surface attachment protein 1 or Sap1) and BPSS1860 (also referred to herein as surface attachment protein 2 or Sap2), were identified and characterized for their role in cellular infection. These proteins are surface antigens used for host-cell attachment (FIG. 2); are conserved in a number of Burkholderia species including Bp, B. mallei and B. cenocepacia, based on all available genomic sequences in NCBI and Burkholderia Genome Database (FIG. 9 and FIG. 10); expressed during infection; and required for complete pathogenesis (FIG. 1). See Example 1. Accordingly, these virulence factors were selected for the development of melioidosis subunit vaccines and monoclonal antibodies (mAb)/nanobodies (Nb) immunotherapies because they are expressed on the surface of Burkholderia bacteria during infection, required for complete pathogenesis, and highly conserved in across Burkholderia species.

Construct Optimization for BPSL0097 Expression. BPSL0097 (SEQ ID NO:4) is a membrane-associated protein with high GC content DNA sequence. To purify BPSL0097 in high quantity and purity, a number of modifications were tried and the following modifications were found to provide for ideal expression and purification. In particular, the DNA sequence encoding BPSL0097 was optimized for E. coli protein expression systems and set forth herein in SEQ ID NO:92; the signal peptide (MKRTGLFLALTGGIVAFSVAQA; SEQ ID NO:93) was excluded; and a minimum of three additional Alanines were added to the C-terminus of BPSL0097 for the final step of purification and successful cleavage from Intein-CBD protein. The sequence of the purified protein is set forth herein in SEQ ID NO:94.

BPSL0097-3×Ala Protein Purification Protocol. pTXB1-BPSL0097-3×Ala plasmid (SEQ ID NO:95) was isolated from strain E3264. This plasmid (200-300 ng) was transformed into chemical competent 5R2566/pLysS_RIPL (strain 53072) E. coli and cells were plated on LB+Ap100+Sp25+Ch125 (100 μg/mL ampicillin, 25 μg/mL streptomycin, and 25 μg/mL chloramphenicol). After an overnight incubation at 37° C., 10-20 colonies were picked and inoculated into 4 mL LB+Ap100+Sp25+Ch125 liquid media. Cultures were allowed to grow to mid-log phase, OD₆₀₀˜1.0, and a small amount of culture was added into one L pre-warmed LB medium with a proper concentration of antibiotics, LB+Ap100+Sp25+Ch125. The culture was incubated at 37° C. with agitation (225 rpm) until the culture reached OD₆₀₀ 0.6-0.7. Subsequently, the temperature was reduced to 20° C. and 0.25 mM IPTG (isopropyl β-D-1-thiogalactopyranoside) was added once the culture reached OD₆₀₀ 0.8-0.9. After an 18-hour induction, cells were pelleted at 8300 rpm for 8 minutes at 4° C. and re-suspended in 30 mL column buffer+1 mM phenylmethylsulfonyl fluoride (PMSF)+0.2% TRITON™ X-100+(optional) 0.5 mM EDTA. The suspension was frozen at −80° C. and the cells were subjected to multiple cycles of freeze-and-thaw to lyse the cells. Sonication was also performed to facilitate cell lysis. DNaseI (10 μg/mL) and MgCl₂ (10 mM) were added to the lysed cells and the mixture was centrifuged at 27,000 rpm for 2 hours at 4° C. Approximately, 10 L of E. coli culture yielded approximately 10-11 mg of BPSL0097 protein. Protein was purified by column chromatography.

Construct Optimization for BPSS1860 Expression. BPSS1860 (SEQ ID NO:12) is a membrane-associated protein with high GC content DNA sequence. To purify soluble BPSS1860, a number of modifications were tried and the following modifications were found to provide for ideal expression and purification. In particular, the DNA sequence encoding BPSS1860 included its own ribosomal binding site and signal peptide (MKFNCIAATILAAVAADATA; SEQ ID NO:96) and is set forth herein in SEQ ID NO:97; and the 6×His-2×Gly tag was needed at the C-terminus of BPSS1860 for the final step of purification, utilizing immobilized metal (divalent nickel) affinity chromatography. The sequence of the purified protein is set forth herein in SEQ ID NO:98.

pMLBAD-BPSS1860-C-His×6 Protein Purification Protocol. pMLBAD-BPSS1860-C-His×6 plasmid (SEQ ID NO:99) was isolated from strain E3040. The plasmid (200-300 ng) was transformed into chemical competent E. coli strain E3072 and cells were plated on LB+Tp100+Sp25+Ch125. Cell culture and induction conditions were the same as those described for BPSL0097. After an 18-hour induction, cells were pelleted at 8300 rpm for 8 minutes at 4° C. and re-suspended in 120 mL column buffer+1 mM PMSF+0.5% TRITON™ X-100+0.1 mg/mL lysozyme. The suspension was frozen at −80° C. and the cells were subjected to multiple cycles of freeze-and-thaw to lyse the cells. Sonication was also performed to facilitate cell lysis. DNaseI (10 μg/mL) and MgCl₂ (10 mM) were added to the lysed cells and the mixture was centrifuged at 27,000 rpm for 2 hours at 4° C. Approximately, 1 L of E. coli culture yielded approximately 0.5 mg of BPSS1860 protein. Protein was purified by column chromatography.

Primary and Booster Vaccine Formulation. Vaccine formulations were prepared as provided in Table 5.

TABLE 5

| Component | Adjuvant Control | Vaccine | |
|---|---|---|---|
| MPLA (1 mg/mL) | 120 μl | 120 | μl |
| Alum (10 mg/mL) | 231 μl | 231 | μl |
| BPSL0097-3xAla (8 mg/mL) | — | 37.5 | μl |
| BPSS1860-His (1.55 mg/mL) | — | 193.5 | μl |
| 1x PBS | 349 μl | 128 | μl |
| Total Volume | 700 μl | 700 | μl |

MPLA, Monophosphoryl-Lipid A

The mixtures were pipetted up and down for 5 minutes to mix and allow the Alum to absorb antigens. Subsequently, 100 μl of adjuvant control or vaccine were administered via i.p. to each mouse.

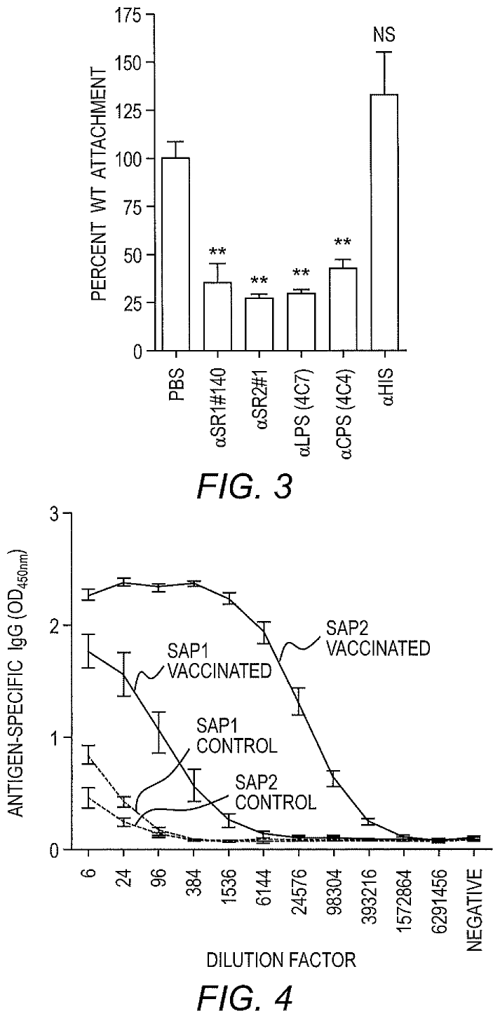

Generation of Antibodies. Monoclonal antibodies (mAbs) and nanobodies (Nbs) were created to assess the viability of Sap1/Sap2-based immunotherapies. First, one mAb against Sap1 (αSR1 #140) and one mAb against Sap2 (αSR2 #1) were generated and binding affinity was demonstrated via immunoblot, which showed specificity of each antibody toward its respective target. To evaluate their potential viability as an immunotherapy, the ability of each mAb to block Bp attachment to HEK293T cells was evaluated. Bp attachment was reduced by either mAb to less than 50% indicating the therapeutic potential of Sap1/Sap2-based immunotherapies (FIG. 3). The observed reduction in attachment provided by αSR1 #140 and αSR2 #1 was comparable to other mAbs targeting other Bp surface structures (FIG. 3, capsular polysaccharide-specific 4C4 and LPS-specific 4C7 monoclonal antibodies; Nualnoi et al. (2017) Am. J. Trop. Med. Hyg. 96(2):358-367). These results indicate the potential of Sap1/Sap2-based mAb immunotherapies to defend against Bp infection.

Nbs are known for their small size, ease of purification, and ability to bind masked epitopes. Therefore, three Nbs targeting Sap1 and two Nbs targeting Sap2 were generated against purified Sap1 and Sap2 (Table 6). These Nbs were shown to bind to their respective targets with a high degree of specificity.

TABLE 6

| | | SEQ ID |
|---|---|---|
| Nanobody | Sequence | NO: |
| NbSap1_1F | MKYLLPTAAAGLLLLAAQPAMAQVQLQESGGGLVQA | 100 |
| | GGSLRLSCEATGRTISSYAMGWFRQAPGKEREFVAA | |
| | INLSSSSTSYADSVKGRFTISRDNAKNTVYLQMNSL | |
| | KPEDTAVYTCADGSNWGQGTQVTVSS | |
| NbSap1_1H | MKYLLPTAAAGLLLLAAQPAMAQVQLQESGGGLVQA | 101 |
| | GGSLRLSCAASGRTFSRYAMGWFRQAPGKEREFVAA | |
| | ISTSGGNTGYADSVKGRFTISRDNAKNTVYYLEMTS | |
| | LKPEDTAVYYCNARRSVGSMSASNFASWGQGTQVTV | |
| | SS | |
| NbSap1_E12 | MKYLLPTAAAGLLLLAAQPAMAQVQLQESGGGLVQA | 102 |
| | GGSLSLSCTASRRTFSSYALGWFRQAPGKEREFVAA | |
| | ISWGGSTTHYADSVQGRFAISRDNAKNTVYYLQMNN | |
| | LKPEDTAVYYCTNGAYWGQGTQVTVSS | |
| NbSap2_D7 | MKYLLPTAAAGLLLLAAQPAMAQVQLQESGGGLVQA | 103 |
| | GGSLRLSCAASGSRFRENAMGWYRQAPGKQRELVAS | |
| | ITSGDSTVYADSVKGRFTISRHNANNTAYAYLQMNN | |
| | LKAEDTAVYYCRHQGSHRYPTVFEYDYWGQGTQVTV | |
| | SS | |
| NbSap2_F7 | MAQVQLQESGGGSVQAGGSLRLSCAASGSIFRENAM | 104 |
| | GWYRQAPGKQREFVADIASGGSTNYADSVKGRFAIS | |
| | RDNAKNTVYYLQMNNLKAEDTAVYYCRHQGSYRRPT | |
| | AYEYEYWGQGTQVTVSS | |

CDRs are underlined

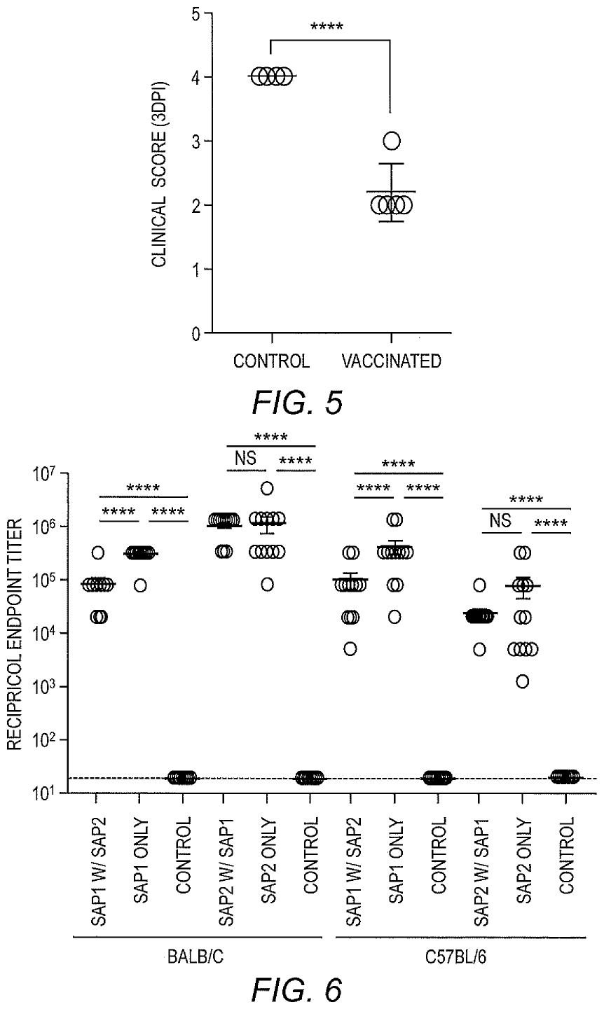

Subunit Vaccines. In addition to Sap1/Sap2-based immunotherapies, Sap1/Sap2-based subunit vaccines were developed. The vaccine composition containing equal concentrations of Sap1 and Sap2 proteins as well as Alum plus MPLA as an adjuvant were administered to BALB/c mice in a primary vaccination followed by a homologous booster two weeks later. Following the booster, mice were bled and sera tested for Sap1- or Sap2-specific IgG via ELISA. All vaccinated mice showed significant Sap1- and Sap2-specific IgG responses when compared to control mice (FIG. 4). Mice were challenged with a lethal dose of Bp 1026b two weeks after the boost and clinical signs and symptoms were tracked for 30 days. At three days post-challenge, all control mice showed a significant degree of clinical symptoms compounded by significant weight loss and were deemed moribund (FIG. 5). Organs of control mice confirmed significant Bp colonization of the lungs and spreading of the infection to other organs. Although vaccinated mice showed weight loss, they also showed less severe clinical symptoms and quickly recovered by day 5 post-challenge. Vaccinated mice survived the entire length of the study highlighting the significance of the Sap1/Sap2-based subunit vaccine. Significantly, vaccinated mice showed very little Bp colonization in the lungs, liver, and spleen with one mouse showing sterile immunity.

To demonstrate the effectiveness of each protein alone as a subunit vaccine, mice were immunized with a Sap1 only subunit vaccine or a Sap2 only subunit vaccine. In particular, Balb/c mice were vaccinated with a Sap1 plus Sap2 vaccine, Sap1 only vaccine, Sap2 only vaccine, or adjuvant control. Antigen-specific IgG titers were determined via ELISA and it was observed that Balb/c mice showed robust IgG responses targeting Sap1 in both vaccine formulations. By comparison, control mice showed no response against Sap1.

Unlike most commercial clinical assays, positive standards are often unavailable at the beginning of an experimental immunization study or when anti-sera are raised against unknown substances. Endpoint titer allow for a quantitative analysis of an unknown sample using negative instead of positive standards. The endpoint titer of a sample is defined as the reciprocal of the highest dilution that gives a positive reaction. To determine whether a reaction is positive or negative, an absorbance cutoff value must be defined. Readings above the cutoff are considered positive while readings at or below the cutoff are negative.

Reciprocal endpoint titers showed a significant difference between Sap1-specific IgG in the control and both vaccine formulations (Sap1 plus Sap2, and Sap1 only) (FIG. 6). In addition, there was a significant difference in Sap1-specific IgG between the Sap1 plus Sap2 vaccine and the Sap1 only vaccine (FIG. 6). Like Sap1, Balb/c mice showed a robust IgG response targeting Sap2 in both vaccine formulations (Sap2 plus Sap1, and Sap2 only). Again, control mice showed no response against Sap2. Reciprocal endpoint titers showed significant difference between Sap2-specific IgG in the control and both vaccine formulations (Sap1 plus Sap2, and Sap1 only) (FIG. 6).

To demonstrate that the observed antibody response was not specific to Balb/c mice, C57Bl/6 mice were vaccinated with a Sap1 plus Sap2 vaccine, Sap1 only vaccine, Sap2 only vaccine, or adjuvant control and antigen specific (Sap1 or Sap2) IgG titers were determined via ELISA. C57Bl/6 mice also exhibited a robust IgG response targeting Sap1 in both vaccine formulations, whereas control mice showed no response against Sap1. Reciprocal endpoint titers showed a significant difference between Sap1-specific IgG in the control and both vaccine formulations (Sap1 plus Sap2, and Sap1 only)(FIG. 6). In addition, there was a significant difference in Sap1-specific IgG between the Sap1 plus Sap2 vaccine and the Sap1 only vaccine (FIG. 6). C57Bl/6 mice further showed a robust IgG response targeting Sap2 in both vaccine formulations. Control mice showed no response against Sap2. Reciprocal endpoint titers showed a significant difference between Sap2-specific IgG in the control and both vaccine formulations (Sap1 plus Sap2, and Sap1 only).

These results demonstrate the use of Sap1/Sap2-based subunit vaccines to induce an immune response against Bp thereby providing complete protection against lethal Bp challenge in the highly susceptible BALB/c model of murine melioidosis.

### Example 3: Sap1 is Required for Pathogenesis of Burkholderia cepacia Complex Strains

### Materials and Methods

Bacterial Strains, Plasmids, Cell Lines, and Growth Conditions. All strains and vectors used and generated in this study are listed in Tables 7 and 8.

TABLE 7

| | | | |
|---|---|---|---|
| Strain | Lab ID | Genotype/Description | Source |

Bcc

| | | | |
|---|---|---|---|
| B. cepacia LO6 | P0754 | Prototroph; cystic | TomLin et al. |
| | | fibrosis isolate | (2001) Can. |
| | | | J. Microbiol. |
| | | | 47: 949-954 |
| LO6 Δsap1 | E4484 | Δsap1::FRT-Tp^{r}- | This work |
| | | pheS/attTn7::miniTn7- | |
| | | gat-rfp | |
| LO6 Δsap1/comp | E4485 | Δsap1::FRT-Tp^{r}- | This work |
| | | pheS/attTn7::miniTn7- | |
| | | Cm-tel-gfp-sap1_L06 | |
| B. cenocepacia | E1554 | Prototroph; cystic | P. Sokol |
| K56-2 | | fibrosis isolate | |
| K56-2 Δsap1 | E3237 | Δsap1::FRT-Tp^{r}- | This work |
| | | pheS/attTn7::miniTn7- | |
| | | gat-rfp | |
| K56-2 | E4486 | Δsap1::FRT-Tp^{r}- | This work |
| Δsap1/comp | | pheS/attTn7::miniTn7- | |
| | | Cm-tel-gfp-sap1_K56 | |

E. coli

| | | | |
|---|---|---|---|
| DH5α | E0272 | F-φ80dlacZΔM15 | Lab collection |
| | | Δ (lacZYA-argF) U169 | |
| | | endA1 recA1 | |
| | | hsdR17 (rK − mK+) | |
| | | supE44 thi-1 ΔgyrA96 | |
| | | relA1 | |
| EP-Max10B- | E1354 | Kan^{r}, Gm^{r}, F⁻ λ⁻ mcrA | Lab collection |
| pir116 Δasd | | Δ (mrr-hsdRMS-mcrBC) | |
| Δtrp::Gm^{r} mob- | | φ80dlacZ ΔM15 | |
| Kan^{r} | | ΔlacX74 deoR recA1 | |
| | | endA1 araD139 Δ (ara, | |
| | | leu) 7697 galU | |
| | | galKrpsL nupG Tn- | |
| | | pir116-FRT2 | |
| | | Δasd::wFRT | |
| | | Δtrp::Gm^{r}-FRT5 | |
| | | mob[recA::RP4-2 | |
| | | Tc::Mu-Kan^{r}] | |
| EP-Max10B-lacI^{q} | E1869 | F⁻ λ⁻ mcrA Δ (mrr- | Lab collection |
| pir | | hsdRMS-mcrBC) | |
| | | φ80dlacZ ΔM15 | |
| | | ΔlacX74 deoR recAl | |
| | | endAl araD139 Δ (ara, | |
| | | leu) 7697 galU | |
| | | galKrpsL nupG lacI^{q}- | |
| | | FRT8 pir-FRT4 | |
| EP-Max10B- | E2072 | F⁻ λ⁻ mcrA Δ (mrr- | Lab collection |
| ΔdapA::Gm^{r}- | | hsdRMS-mcrBC) | |
| lacI^{q}-pir leu + | | φ80dlacZ ΔM15 | |
| mob-Kan^{r} | | ΔlacX74 deoR recA1 | |
| | | endA1 galU galKrpsL | |
| | | nupG ΔdapA::lacI^{q}- | |
| | | FRT8 pir-Gm^{r} leu + | |
| | | mob[recA::RP4-2 | |
| | | Tc::Mu-Kan^{r}] | |

TABLE 8

| Plasmids | Lab ID | Relevant properties | Source |
|---|---|---|---|
| pRK2013 | E0272 | Kan^{r}, helper plasmid | Figurski & |
| | | encoding conjugative | Helinski (1979) |
| | | proteins | Proc. Natl. Acad. |
| | | | Sci. USA |
| | | | 76: 1648-1652 |
| pPS854-FRT- | E0855 | Ap^{r}; Cm^{r}; plasmid with | Lab collection |
| Cm^{r} | | Cm^{r}-FRT-cassette | |
| pwFRT-P_{CS12}- | E1584 | Tel^{r}; P_{CS12}-Tel^{r} cassette | Barrett, et al. |
| Tel^{r} | | flanked by wild-type | (2008) |
| | | FRT sequences | Appl. Environ. |
| | | | Microbiol. |
| | | | 74: 4498-4508 |
| mini-Tn7- | E2462 | GS^{r}, mini-Tn7-gat | Norris et al. |
| gat-gfp | | harboring gfp | (2009) |
| mini-Tn7- | E2326 | GS^{r}, mini-Tn7-gat | Appl. Environ. |
| gat-rfp | | harboring rfp | Microbiol. |
| | | | 75: 6062-6075 |
| mini-Tn7- | E4487 | Cm^{r}; Tel^{r}; mini-Tn7 | This work |
| Cm-tel-gfp- | | harboring gfp and K56 | |
| sap1_K56 | | sap1 | |
| mini-Tn7- | E4488 | Cm^{r}; Tel^{r}; mini-Tn7 | This work |
| Cm-tel-gfp- | | harboring gfp and LO6 | |
| sap1_LO6 | | sap1 | |
| pTNS3-asd_{Ec} | E2237 | Suicidal helper plasmid | Kang et al. |
| | | containing E. coli asd | (2009) |
| | | and transposase for the | Appl. Environ. |
| | | Tn7 site-specific | Microbiol. |
| | | transposition system | 75: 4015-4027 |
| pKaKa5 | E3217 | Tet^{r}; broad-host-range | Heacock-Kang |
| | | A-red helper plasmid | et al. (2018) |
| | | based on Tet resistance | Sci. Rep. |
| | | that also confers DNA | 8: 12422 |
| | | uptake | |
| pFRT1-Tp^{r}- | E2964 | Tp^{r}, Ap^{r}, FRT1 flanked | Barrett, et al. |
| pheS | | Tp^{r}-pheS cassette | (2008) |
| | | | Appl. Environ. |
| | | | Microbiol. |
| | | | 74: 4498-4508 |

Bcc and E. coli were cultured using LB medium (Difco) or 1×M9 minimal medium supplemented with 20 mM glucose, 500 μM MgSO₄, and 25 μM CaCl₂) (MG), and following additives when appropriate: trimethoprim (Tp; 100 μg/mL for E. coli, 200 μg/mL for K56-2, 300 μg/mL for LO6), tetracycline (Tet; 10 μg/mL for E. coli, 50 μg/mL for K56-2, 150 μg/mL for LO6), tellurite (Tel; 10 μg/mL for E. coli, 125 μg/mL for Bcc), chloramphenicol (Cm; 25 μg/mL for E. coli, 150 μg/mL for Bcc), glyphosate (GS; 0.1% v/v for E. coli, 0.4% v/v for Bcc), chlorinated phenylalanine (cPhe, 0.1% w/v), and diaminopimelic acid (DAP; 100 μg/mL). Derivatives of E. coli strain EP-Max 10B (BioRad), E1869 and E1354 were routinely used for cloning or plasmid mobilization into Bcc as previously described (Kang et al. (2011) Nat. Protoc. 6:1085-1104; Norris et al. (2009) Appl. Environ. Microbiol. 75:6062-6075).

Human lung epithelial cell line A549 and murine macrophage cell line RAW264.7 were cultured in DMEM containing 4.5 g/L glucose with 4.0 mM L-glutamine (HyClone). All cultures were supplemented with 10% (v/v) heat-inactivated standard FBS (HyClone) and antibiotic/antimycotic solution (100 U/mL penicillin, 100 μg/mL streptomycin, and 250 ng/mL amphotericin B; HyClone). Cells were maintained at 37° C. and 5% CO₂ in a humidified incubator. Cell lines were maintained at 50-80% confluence at which point RAW264.7 macrophages were passaged using a cell scraper and A549 cells were passaged with 0.25% trypsin-EDTA (Gibson). Cell lines were maintained in CORNING™ flasks and plates with CELLBIND® surfaces. Cell concentrations were determined using the Scepter handheld automated cell counter (Millipore).

General Molecular Methods. Oligonucleotides were synthesized through Integrated DNA Technology and are listed in Table 9. Ail molecular methods and their components utilized were employed as previously described (Kang et al. (2011) Nat. Protoc. 6:1085-1104).

TABLE 9

| | | SEQ ID |
|---|---|---|
| Oligo ID; Oligo name | Sequence (5′→3′) | NO: |
| 876; TN7L | ATTAGCTTACGACGCTACACCC | 105 |
| 3093; V_C0817 pheS-gat | GATCCACAGCGCAACGGCCCGGCATG | 106 |
| dw | CGCCGGGCCGTGATCGGTTCAGCTGG | |
| | CACGACAG | |
| 3094; V_C0817 pheS-gat | AAACACATAGCTAACCCAGCCTGGTG | 107 |
| up | CCCGCAAGCCTGGAGACAGGGCGATT | |
| | AAGTTGGG | |
| 3095; V_C0817 pheS-M13 | TGCGCGATCCACAGC | 108 |
| dw END | | |
| 3096; V_C0817 pheS-gat | TCACCTTTAAACACATAGCTAAC | 109 |
| up END | | |
| 3097; V_C0817 pheS-gat | GCATTCGGTTCGGTCGG | 110 |
| dw screen | | |
| 3098; V_C0817 pheS-gat | TGTGCTTGCTATCGTTTACCG | 111 |
| up screen | | |
| 3099; glmS1 Bc K56-2 | GTGTGAAACCACTTCGTCTTG | 112 |
| 3100; glmS2 Bc K56-2 | GAAGATCGTGCTCGGCGAAATG | 113 |
| 3101; glmS3 Bc K56-2 | TTCCTGCGTTCGGTGCCAGTCG | 114 |
| 3102; glmS4 Bc K56-2 | CCGAGCTGCTGAAGAACACC | 115 |
| 3166; BamHI V_C0817 | TATATGGATCCGCGGGAATGGACG | 116 |
| miniTn7 | | |
| 3167; PstI V_C0817 | ATATCTGCAGTGGAATCGTTTCGGAT | 117 |
| miniTn7 | G | |
| 3169; LO6 PstI V_C0817 | ATATCTGCAGCGATAAATTCATATCG | 118 |
| miniTn7 | TTTCGGC | |
| 3170; LO6 BamHI | TATATGGATCCGCGCGTTACTGCTTG | 119 |
| V_C0817 miniTn7 | AT | |
| 3214; HindIII V_C0817 | ATATAAGCTTGGAATCGTTTCGGATG | 120 |
| mT7gRFP | | |
| 3215; SpeI V_C0817 | TATATACTAGTCGCGGGAATGGACG | 121 |
| mT7gRFP | | |
| 3216; LO6 HindIII | ATATAAGCTTCGATAAATTCATATCG | 122 |
| V_C0817 mT7gRFP | TTTCGGC | |
| 3217; LO6 SpeI V_C0817 | TATATACTAGTCGCGCGTTACTGCTT | 123 |
| mT7gRFP | GAT | |
| 3297; KpnI V_C0817 dn | TATATGGTACCGCGGGAATGGACG | 124 |
| 3298; KpnI V_C0817 up | ATATGGTACCTGGAATCGTTTCGGAT | 125 |
| | G | |
| 3299; LO6 KpnI V_C0817 | TATATGGTACCGCGCGTTACTGCTTG | 126 |
| dn | | |
| 3300; LO6 KpnI V_C0817 | ATATGGTACCGATAAATTCATATCGT | 127 |
| up | TTCGGC | |
| 3499; LO6 glmS1 | GGTACCGACGTCGACAAGC | 128 |
| 3500; LO6 glmS2 | CGACAAGCCGAGGAATCTGG | 129 |
| 3501; LO6 glmS3 | GCTGCTCGCGTATCACACC | 130 |

Construction of Mutant and Complementation Strains. Bcc strains K56-2 and LO6 were mutated using λ-RED recombineering (Heacock-Kang et al. (2018) Sci. Rep. 8:12422; Kang et al. (2011) Nat. Protoc. 6:1085-1104). Briefly, pKaKa5 was tri-parentally conjugated into each Bcc strain assisted by pRK2013 and selected on MG with appropriate concentrations of tetracycline. For mutant construction, pFRT-Tp-pheS was used as PCR template with oligos 3093-3096 and 1 μg of the product was co-incubated with Bcc strains containing pKaKa5 at room temperature for 30 minutes. The co-incubation mixture was recovered in 4 mL of LB for 4 hours shaking at 225 rpm, after which the culture was harvested and plated on LB+Tp200 (K56-2) or LB+Tp300 (LO6). Resulting colonies were purified on their respective media and screened for proper deletion via PCR. Verified mutants were tagged using the mini-Tn7-gat-rfp plasmid as described (Norris et al. (2010) Appl. Environ. Microbial. 76:7635-7640). Successful integrations were verified with PCR of the four glmS sites and fluorescent microscopy.

Complementation also used the mini-Tn7 based integration method (Norris et al. (2010) Appl. Environ. Microbial. 76:7635-7640; Kang et al. (2009) Appl. Environ. Microbial. 75:4015-4027). Briefly, Bcc genomic DNA was used as template for PCR with respective primers (3297-3300) to amplify the sap1 gene, as well as introducing KpnI digestion sites on either side to be cloned into mini-Tn7-gat-gfp linearized by KpnI. The resulting plasmids, mini-Tn7-gat-gfp-sap1_K56 and mini-Tn7-gat-gfp-sap1_LO6, were then digested with PstI and XhoI to replace the gat gene with a tellurite resistance cassette to differentiate strains in competition assays. Additionally, a Cm^{r} cassette was cloned in by BspHI and BglII digestion of pPS854-ERT-Cm^{r}. Restriction digest verified plasmid size and orientation. Genomic integration of the complementation vectors mini-Tn7-Cm-tel-gfp-sap1_K56 and mini-Tn7-Cm-tel-gfp-sap1_LO6 was done as previously described (Norris et al. (2010) Appl. Environ. Microbiol. 76:7635-7640) and verified by PCR and florescent microscopy.

Growth Characterization of Mutants and Complementation Strains. Wild-type, mutant, and complement strains were grown to stationary phase, sub-cultured 1:200 into 250 mL of LB in a 500 mL flask and grown at 37° C. and 225 rpm. Aliquots of 250 μL were pulled at designated times, added to 750 μL ddH₂O in cuvettes, and absorbance was read via spectrophotometer at 600 nm.

Attachment Assays. The attachment assay was carried out by dilution of bacterial strains in PBS and plated on LB. Colonies were counted to determine the number of colony forming units (CFU) used to initiate the infection and calculate attachment efficiencies. The dilutions were used to infect the cell cultures in 24-well CELLBIND® plates at an MOT of 10:1. After 30 minutes, the bacteria-containing medium was removed and the monolayers were washed 3 times with pre-warmed PBS. Monolayers were lysed with 0.2% TRITON™ X-100 in PBS, diluted, plated onto LB and incubated at 37° C. for 48 hours. Colonies were enumerated and attachment efficiency was determined by dividing the attached number by the initial number of bacteria. The experiment was carried out in triplicate, normalized to each wild-type control, and the numbers represent the average of all three replicates with the error bars representing the standard errors of the mean (s.e.m.). An unpaired t-test was used to determine the significance of attachment efficiencies between the wild-type, mutants, and complements.

Biofilm Crystal Violet Assay. Quantification of biofilm production was performed as previously described with modifications (Merritt et al. (2005) John Wiley & Sons, Inc.; O'Toole (2011) J. Visual. Exp. 2437). Briefly, bacterial strains were grown to late-log, sub-cultured at 1:200, and 125 μL dispensed into Costar SEROCLUSTER™ 96-well microtiter plates (CORNING™) with each strain being replicated ten times. Sterile media served as a negative control. The outside perimeter of the 96-well plates was filled with ddH₂O to minimize evaporation from wells. After 18 hours, wells were rinsed with ddH₂O and patted dry. At this point, half of the wells were filled with 150 μL of 1% (w/v) crystal violet for 15 minutes, rinsed, and allowed to air dry. The other wells were filled with 150 μL of 0.1% (w/v) SDS resuspension mixture and mixed via repeated pipetting and scraping of the sides. The resulting resuspensions were then serially diluted and plated on LB to enumerate CFUs. The crystal violet-stained wells were solubilized with 200 μL of a modified biofilm dissolving solution of 80% ethanol+10% SDS (w/v), and 150 μL transferred to a flat-bottomed 96-well microtiter plate and read for absorbance at 550 nm (Tram et al. (2013) Adv. Microbiol. 3(2):200-204). An unpaired t-test was used to determine the significance of differences in biomass and CFUs between the wild-type, mutants, and complements.

D. melanogaster in vivo Competition Study. D. melanogaster studies were performed as described previously with modifications (Castonguay-Vanier et al. (2011) PLoS ONE 5:e11467). Briefly, wild-type Oregon R flies were maintained on standard cornmeal sucrose medium. Mutant and complement strains were washed in a 5% sucrose+PBS solution and adjusted to a final 1:1 ratio totaling 2.5×10¹⁰ CFU/mL. From this, 150 μL was added to Whatman filters atop 5 mL of 5% sucrose agar and allowed to dry at room temperature for 30 minutes. Twelve, 1- to 3-day old male flies were starved for 3 hours and added to each vial, with three vials for a total of 36 flies used per Bcc strain combination. As a control, the mixtures were also plated on 5% sucrose agar without flies to monitor the ratio over time. At two and four days post-inoculation, three flies from each vial were taken for CFU enumeration, with each vial's flies being treated separately. Serial dilutions of the fly homogenate were plated onto LB (CFUₜₒₜₐₗ) or LB with chloramphenicol and tellurite (CFU_{complement}). CFUₘᵤₜₐₙₜ was determined from the difference of CFUₜₒₜₐₗ and CFU_{complement} the ratio of which to yields the in vivo competitive index (CI) (CFUₘᵤₜₐₙₜ/CFU_{complement}).

Imaging of the Gastrointestinal Tract and Crop. Additional flies from the above CI experiments were sacrificed; the crops and gastrointestinal tract (GI) were carefully removed under a dissection microscope and cured in PROLONG™ Gold Antifade reagent (Invitrogen) as previously described (Heacock-Kang et al. (2017) Mol. Microbiol. 106:976-985). A cover slip was placed over the organs and pressed flat to remove air bubbles and sealed with clear nail polish applied along the edges. After letting cure for 30 minutes, the slides were imaged using a Zeiss Observer D1 with AxioCam MRc5 and accompanying Axiovision 4.9.1 software.

C. elegans Survival Studies. Each bacterial strain was inoculated on three plates with nematode growth media (NGM) and allowed to grow as lawns for 36 hours; fifteen L4 age-synchronized C. elegans were added to each plate for a total of 45 worms for each Bcc strain (Tan et al. (1999) Proc. Natl. Acad. Sci. USA 96:2408-2413). E. coli OP50 was used as a food source as well as a negative control. Observations were recorded daily. The experiment was conducted twice with comparable results.

BALB/c Mice Infection Studies. Frozen aliquots of the strains were plated to determine accurate CFUs. Bacteria used for inoculations were washed in PBS, diluted to the desired concentration, and plated at the start of the study to accurately determine the number of CFUs used. Six-week-old female BALB/c mice were purchased from Jackson Laboratories. Before challenge, the mice were anesthetized by intraperitoneal injection of 100 mg/kg ketamine and 10 mg/kg xylazine. For the CI studies, 40 μL of the 1:1 mutant/complement (7×10⁷ CFU/mouse) strain mixture was inoculated intratracheally using the BioLITE Intubation System (Braintree Scientific), with PBS as negative control. Five mice were used per group. The same 1:1 mixture was also plated for comparative CFU changes as a further control. After 3 or 5 days, mice were euthanized after which their lungs were harvested and homogenized in 5 mL PBS. Serial dilutions of the homogenate were plated onto LB (CFUₜₒₜₐₗ) or LB with chloramphenicol and tellurite (CFU_{complement}). CFUₘᵤₜₐₙₜ Was determined from the difference of CFUₜₒₜₐₗ and CFU_{complement} the ratio of which to yields the in vivo CI (CFUₘᵤₜₐₙₜ/CFU_{complement}).

For survival studies, mice were inoculated via the intratracheal route with 3×10⁸ CFU/mouse with five mice used per strain. One mouse in LO6 Δsap1 group died during intubation and was censored from the experiment. Survival was monitored for 10 days, and moribund mice were euthanized according to predetermined criteria for end point. At the end of 10-day post-infection, surviving mice were euthanized, and the lungs, livers, and spleens were homogenized in 5 mL PBS for serial dilution and CFU enumeration.

### Results and Discussion

Sap1 is Conserved in Burkholderia Species Including Bcc K56-2 and Bcc LO6. Sap1 (BPSL0097) is a Bp attachment factor that is expressed on the poles of the bacterial surface and required for complete pathogenesis. See Example 1. Within the Burkholderia species there are 627 orthologs of Sap1 (Burkholderia Ortholog Group BG002978) indicating a conserved function within the genus. Within the ortholog group, approximately 42% of the strains are within the Bcc, including B. cenocepacia K56-2 and B. cepacia LO6 isolated from CF patients. There is a high similarity between the Bp K96243, Bcc K56-2, and Bcc LO6 genetic loci of sap1. The surrounding genes are organized similarly with an acetyltransferase upstream (BSPL0096, RS04965, and RS04845) and a hypothetical protein downstream (BPSL0098, RS04975, and RS04855) of each sap1 homolog. There is conservation at the nucleotide level in this genetic region with 68% identity between Bp K96243 and both Bcc strains while there is 82.3% identity between Bcc strains. In addition, the Sap1 protein sequence is conserved between Bp K96243, Bcc K56-2, and Bcc LO6 strains. The Sap1 homologs in Bcc K56-2 (SEQ ID NO:8) and Bcc LO6 (SEQ ID NO:9) have 81.6% and 84.2% amino acid identity with Sap1 from Bp K96243 (SEQ ID NO:4), respectively, and 93% amino acid identity with each other.

While PSORTb3.0 analysis showed that Sap1 homologs have an unknown cellular localization (Winsor et al. (2008) Bioinformatics 24:2803-2804), the results presented in Example 1 indicate that Sap1 protein is exported and expressed on the bacterial surface. Sap1 export is likely through a standard Sec-dependent pathway indicated by the N-terminal signal peptide and predicted cleavage site between amino acids 22-23 (probability of 0.8729) in Bp K96243 Sap1 determined through SignalP 5.0 analysis (Almagro Armenteros et al. (2019) Nat. Biotechnol. 37:420-423). Bcc K56-2 and Bcc LO6 have similar predicted cleavage sites between amino acids 23-24 (probabilities of 0.9083 and 0.9444) indicating similar export. Approximately 46% of the amino acid changes between the Sap1 homologs is in the N-terminal signal peptide. Accordingly, when the signal peptide sequence is excluded, Bcc K56-2 and Bcc LO6 Sap1 homologs share 87% and 89.1% amino acid identity with Bp K96243 Sap1 and 95.7% amino acid identity with each other. Taken together, these observations indicate a conserved function of Sap1 across Burkholderia species.

Sap1 is Required for Biofilm Formation and Attachment to Host Cells. To investigate the role of Bcc Sap1 in attachment to non-living and living surfaces, deletional mutants of the sap1 gene were generated in Bcc strains K56-2 and LO6 using λ-RFD recombineering and corresponding complements were prepared using the mini-Tn7-based integration system. The Δsap1 mutants and complements showed no defects during in vitro growth when compared to the wild-type strains, K56-2 or LO6.

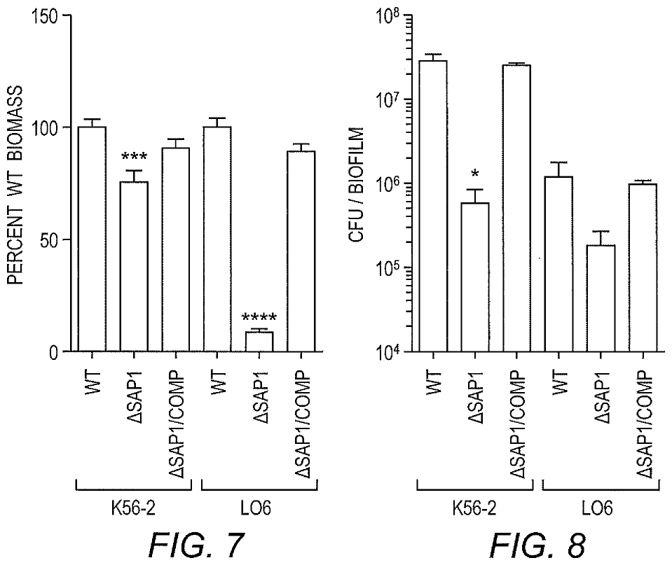

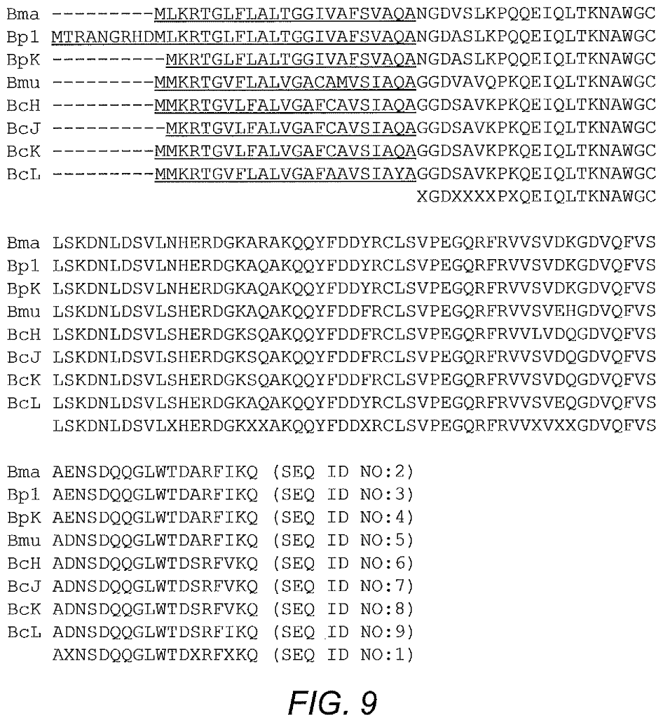

Since attachment is an important initial step in biofilm formation, a biofilm assay was used to determine whether the Δsap1 mutants were defective in biofilm formation. The K56-2 Δsap1 mutant was modestly reduced in its ability to form biofilm and the complement recovered this defect (FIG. 7). Within the biofilm architecture, reduced CFUs of the K56-2 Δsap1 mutant were found (FIG. 8). Similarly, the LO6 Δsap1 mutant showed a major decrease in biofilm formation and biofilm CFOs when compared to the wild-type LO6 strain, and these defects were complemented back to wild-type levels (FIG. 7 and FIG. 8). Both Δsap1 mutants showed statistically significant reductions in biofilm formation. The biofilm formation defects seen in both Δsap1 mutants indicated a conserved role of Sap1 in Bcc strains.

To further characterize the role of Bcc Sap1 on biological surfaces, the Δsap1 mutants were tested for their ability to attach to A549 human lung epithelial cells and RAW264.7 murine macrophage cells. The K56-2 Δsap1 mutant was able to attach to A549 and RAW264.7 cells at 13% and 22% of wild-type K56-2 level. Complementation of the K56-2 Δsap1 mutant rescued this defect indicating that Sap1 is involved in the attachment process. The LO6 Δsap1 mutant was also significantly defective in attachment to both cell lines. The LO6 Δsap1 mutant showed an attachment ability at 30% of wild-type LO6 to A549 cells and at 2% of wild-type LO6 to RAW264.7 cells. The complementation of the LO6 Δsap1 mutant restored its attachment ability to wild-type levels. The Sap1 protein is confirmed here to be essential for attachment of K56-2 and LO6 strains to different host cell types, indicating its significant role in this initial step of Bcc pathogenesis.

The Δsap1 Mutants are Less Competitive in D. melanogaster. Given the importance of the Sap1 protein in biofilm formation and host cell attachment, it was determined whether Sap1 is involved in in vivo fitness, colonization, and pathogenesis within invertebrate and vertebrate hosts. The D. melanogaster feeding model, a well-established invertebrate model useful in assessment of in vivo bacterial fitness, was used to determine if Sap1 is important for colonization of the fly crop (Sibley et al. (2008) PLoS Pathog. 4:e1000184; Castonguay-Vanier et al. (2011) PLoS ONE 5:e11467; Mulcahy et al. (2011) PLoS Pathog. 7:e1002299). Equal CFUs of each Δsap1 mutant and its corresponding complement strain were mixed for infection to determine the CI in D. melanogaster. The CI is the ratio of the mutant to its complement and is a metric representing the fitness of the Δsap1 mutant within a specific environment. A CI of less than one indicates that the mutant is less competitive than its complement strain. When grown together in LB medium, the K56-2 Δsap1 mutant and its complement strain had an average CI of 0.99 and 0.96 at two and four days post-infection (dpi), respectively, indicating no fitness difference in vitro. The K56-2 Δsap1 mutant and its complement strain had an average CI of 0.33 and 0.28 at two and four dpi in D. melanogaster, indicating a significant defect of the Δsap1 mutant in in vivo fitness compared to the complemented strain. This quantitative evidence was corroborated by qualitative fluorescent microscopy of fly crops showing that the gfp-tagged complement strain colonized at higher levels than the rfp-tagged Δsap1 mutant. A similar trend was observed with the LO6 strain. The LO6 Δsap1 mutant showed no defects in its ability to compete with its complement strain in vitro, with CIs of 1.01 and 1.05 at two and four dpi. At two dpi, the LO6 Δsap1 mutant and complement have a CI of 0.34, indicating that the complement out competed the mutant significantly. At four dpi, the CI was reduced to 0.17 further highlighting the defect of the LO6 Δsap1 mutant during in vivo colonization. This was confirmed qualitatively through visualization of the D. melanogaster crop showing a high proportion of the gfp-tagged complement strain compared to the rfp-tagged Δsap1 mutant. These results show that Sap1 is important for in vivo fitness of these two Bcc strains in the fly feeding model and facilitates colonization of the fly crop.

Sap1 is Required for Pathogenesis in vivo in C. elegans. It was subsequently determined whether defects in colonization mediated by Δsap1 are correlated with changes in Bcc virulence. To assess the importance of Sap1 in vivo, the K56-2 and LO6 Δsap1 mutants were tested in a C. elegans survival assay. C. elegans (n=45) were allowed to feed on lawns of the wild-type strains, Δsap1 mutants, or the complement strains and survival was monitored for 218 hours. The C. elegans feeding on the K56-2 wild-type strain had a gradual decrease in survival leading to 100% mortality at 192 hpi. The Δsap1 mutant showed a significant decrease in virulence, resulting in significantly less killing of C. elegans when compared to the wild-type K56-2 strain. Complementation restored virulence to wild-type levels. Similarly, the LO6 Δsap1 mutant was highly attenuated in C. elegans, and the complemented strain was restored to wild-type killing level. The wild-type LO6 strain was more virulent than the K56-2 strain and showed 100% mortality by 96 hpi, indicating some variation in pathogenesis between the two strains in this animal model. Taken together, the data indicate that Sap1 is a critical virulence factor for the pathogenesis of K56-2 and LO6 in vivo in C. elegans.

The LO6 Δsap1 Mutant is Attenuated in BALB/c Mice. Involvement of Sap1 in Bcc colonization and virulence in a more relevant vertebrate infection model (e.g., BALB/c mouse lung infection model) was subsequently determined. BALB/c mice were inoculated by intratracheal intubation to assay the CI of the Δsap1 mutant and its complement. In a pilot study, the K56-2 wild-type strain was not able to colonize the BALB/c lungs, and therefore was excluded from further examination. Inoculation was done with mixture of an equal amount of LO6 Δsap1 mutant and its complement, at a total inoculum of 7×10⁷ CFU/mouse. The CI at three dpi showed a five-fold decrease in fitness of the LO6 Δsap1 mutant with an average CI of 0.21. The CI was further reduced at day five-post infection to 0.1, translating to a 10-fold decrease in fitness. These data indicate that the complement was able to outcompete the Δsap1 mutant when colonizing the BALB/c lung.

Following the competitiveness assessment of the LO6 Δsap1 mutant, a survival study was then performed to determine if the LO6 Δsap1 mutant was attenuated. BALB/c mice were challenged by intratracheal intubation with an inoculum of ˜3×10⁸ CFU/mouse of the LO6 wild-type strain, the LO6 Δsap1 mutant, or its complement. Compared to the 7×10⁷ CFU/mouse in the CI study, the higher inoculum for the survival study was determined from a pilot study to induce mortality. Seventy-five percent of mice challenged with the LO6 Δsap1 mutant (n=4) survived the duration of the study. On the other hand, BALB/c mice challenged with the wild-type LO6 strain (n=5) or the Δsap1 complement (n=5) showed 20% and 40% survival, respectively. The lungs, livers, and spleens of the surviving mice were harvested to determine dissemination and bacterial burdens. Various levels of bacteria were recovered from all organs, except for the liver from one Δsapl mutant infected mouse. The lungs from the mice challenged with the LO6 wild-type and complement strain retained a bacterial load that was above or comparable to the initial inoculum. This indicated that LO6 was able to replicate and persist in the mouse lungs, while also disseminating to other organs. The LO6Δsap1 mutant colonized the lungs at a lower concentration than the LO6 wild-type and complement, indicating the involvement of Sap1 protein in Bcc attachment and initial colonization. However, even with the lower level of lung colonization, the LO6 Δsap1 mutant was able to disseminate to other organs at comparable levels to the wild-type and complement strains, suggesting that Sap1 is not critical for the subsequent dissemination process. Nonetheless, it is important to note that this reduction of the LO6 Δsap1 mutant in colonization in the mouse lungs led to lower virulence and fatality in BALB/c mice. Taken together, these data indicate that Sap1 is involved in Bcc colonization of BALB/c mouse lungs and required for complete pathogenesis.

### Example 4: Live-Attenuated Vaccines

### Materials and Methods

Bacterial Strains, Media and Culture Conditions. Bp strain 1026b was routinely grown in LB medium (Difco) or 1× M9 minimal medium supplemented with 20 mM glucose (MG) and with 0.3% glyphosate when appropriate.

Molecular Methods and Reagents. Molecular methods and reagents were used as described previously (Norris et al. (2011) Infect. Immun. 79:4010-4018; Kang et al. (2011) Nat. Protoc. 6:1085-1104; Norris et al. (2010) Appl. Environ. Microbiol. 76:7635-7640; Norris et al. (2009) Appl. Environ. Microbiol. 75:6062-6075. Mutants of genes BPSL0636, BPSS0015 and BPSS1818 were generated previously. Double mutant combinations were generated by using Flp recombinase to remove the glyphosate resistance gat-cassette, followed by PCR verification, and lambda red recombineering to generate the second deletion.

Animal Studies. BALB/c and C57BL/6 mice between four and six weeks of age were purchased from Charles River Laboratory. All vaccinations and challenges were administered via the intranasal (i.n.) inoculation route. Mice were anesthetized with a mixture of 100 mg ketamine and 10 mg xylazine/kg as described herein. Each mutant strain was used to vaccinate five mice per group with 4,500 CFU or 12,000 CFU for BALB/c and C57BL/6 mice, respectively. Primary vaccinations were carried out 30 or 60 days prior to challenge with wild-type Bp 1026b. Booster vaccinations were carried out 30 days prior to challenge. Vaccinated mice were challenged with 4,500 CFU (BALB/c) or 12,000 CFU (C57BL/6) of wild-type Bp 1026b. Animals were monitored for disease symptoms daily and euthanized at predetermined humane end points. Clinical scores are as follows: 1-questionable illness, 2-mild illness, 3-moderate but definitive illness, 4-severe illness-moribund (pre-determined humane end point), 5-found dead. Lungs, liver, and spleen of surviving mice were harvested, homogenized, serially diluted, and plated on LB and MG with glyphosate. Growth on LB represents total bacteria within each organ and MG represents any surviving mutant strain (gat+). Survival data was plotted using Prism software (GraphPad, La Jolla, CA) and statistical analysis carried out by Log-rank (Mantel-Cox) test.

### Results and Discussion

Virulence factors BPSL0636, BPSS0015, and BPSS1818 encode a protein of unknown function, a protein involved in autophagy evasion, and a protein involved in host tubulin rearrangement, respectively. As demonstrated in Example 1, a mutant of BPSL0636 shows a 29% decrease in intracellular replication and 56% decrease in cell-to-cell spread at 24 hours when compared to wild-type Bp. A BPSS0015 mutant is able to replicate at or above wild-type levels during early stages of infection, but has an 85% decrease in intracellular replication and 38% decrease in cell-to-cell spread at 24 hours post infection. By comparison, BPSS1818 is involved in host cytoskeletal structure modifications, leading to an 84% decrease in intracellular replication and 46% decrease in cell-to-cell spread of the BPSS1818 mutant. Further, when BALB/c mice are infected with strains carrying a single mutation of BPSL0636, BPSS0015, or BPSS1818, all mice survived for 60 days, while BALB/c mice infected with an equivalent dose of the wild-type Bp 1026b are unable to survive beyond day five. Taken together, these data show that these virulence factors are differentially expressed during intracellular infection, important for intracellular survival, and single mutants of these virulence factors are attenuated in BALB/c mice. Accordingly, knockout mutants of these genes were tested for use as live-attenuated vaccines against Bp infection.

These virulence factors are highly conserved in Bp strains as well as other Burkholderia pathogens, including Bm and the B. cepacia complex (Bcc), the causative agents of glanders and cepacia syndrome, respectively (Table 10).

TABLE 10

| | | Percent Amino Acid Identity | | |
|---|---|---|---|---|
| | | Compared to Bp K96243 | | |
| Genus species | Strain | BPSL0636 | BPSS0015 | BPSS1818 |
| B. pseudomallei | K96243 | 100 | 100 | 100 |
| B. pseudomallei | 1026b | 100 | 100 | 100 |
| B. pseudomallei | MSHR487 | 100 | 100 | 100 |
| B. pseudomallei | MSHR503 | 100 | 100 | 99.54 |
| B. pseudomallei | NAU44A6 | 99.27 | 99.35 | 99.54 |
| B. pseudomallei | MSHR840 | 100 | 99.35 | 98.62 |
| B. pseudomallei | MSHR1655 | 99.27 | 100 | 99.54 |
| B. pseudomallei | MSHR87 | 99.27 | 100 | 99.54 |
| B. mallei | ATCC23344 | 0 | 100 | 0 |
| B. mallei | Ivan | 100 | 100 | 0 |
| B. mallei | China 5 | 100 | 100 | 0 |
| B. cenocepacia | J2315 | 83.21 | 68.18 | 77.88 |
| B. cenocepacia | K56-2 | 83.21 | 68.18 | 77.88 |
| B. cepacia | LO6 | 83.21 | 68.83 | 79.26 |
| B. multivorans | ATCC 17616 | 83.94 | 65.58 | 77.42 |
| B. vietnamiensis | G4 | 83.21 | 69.48 | 78.34 |
| B. ubonensis | A21 | 82.48 | 66.23 | 78.34 |
| B. oklahomensis | C6786 | 91.97 | 87.01 | 95.39 |
| B. thailandensis | E264 | 95.62 | 94.07 | 97.24 |

Within the Burkholderia genus, BPSL0636, BPSS0015, and BPSS1818 have 652, 642, and 638 ortholog group members, respectively. Among a small set of Bp strains, conservation of all three proteins is observed with 98-100% amino acid identity (Table 10). BPSL0636 is present in Bm strains Ivan and China 5 at 100% identity and also showed strong conservation in Bcc and other pathogenic Burkholderia species (Table 10). BPSS1818 is absent in Bin but highly conserved in Bcc (77-80% amino acid identity) and other Burkholderia species (95-97% amino acid identity), indicating that it plays a role in the pathogenesis of multiple Burkholderia species (Table 10). The high level of conservation among a diverse group of pathogenic Burkholderia species indicates that these virulence factors have conserved function.

Vaccination with Virulence Factor Mutants Partially Protects Against Lethal Bp Challenge. To test the efficacies of these mutants as potential live-attenuated vaccines, BALB/c mice were used as a host in an infection model of melioidosis (Muller et al. (2012) Infect. Immun. 80:3247-3255). Using this model, unvaccinated mice infected with virulent wild-type Bp show signs of disease quickly, leading to 100% mortality within the first week post-infection (Norris et al. (2011) Infect. Immun. 79:4010-4018; Propst et al. (2010) Infect. Immun. 78:3136-3143). Groups of five mice were first vaccinated via the intranasal (i.n.) route with a single 4,500 CFU dose of each attenuated virulence factor mutant, as well as PBS as control, and monitored for 60 days. During these 60 days, vaccinated mice showed no significant sign of illness, comparable to PBS control mice. At day 60, mice were challenged (i.n.) with a lethal dose of wild-type Bp 1026b (4,500 CFU) and survival was monitored for 63 days. Within the first six days of challenge, control mice vaccinated with PBS showed signs of disease and were euthanized at predetermined humane end points. In contrast, the mice vaccinated with the BPSL0636 mutant showed 60% survival for the duration of the study, while 40% of the mice vaccinated with the BPSS0015 or BPSS1818 mutants survived the wild-type challenge. One mouse vaccinated with the BPSL0636 mutant showed a delay in mortality to day 22. All of the surviving mice were euthanized at the end of the study and their organs were harvested for bacterial burden determination. Of the surviving mice, some showed colonization of wild-type Bp in the lungs combined with dissemination to the liver and/or spleen —. A single mouse vaccinated with the BPSL0636 mutant strain showed sterile immunity with no signs of infection in the lungs, liver, or spleen. Although mice vaccinated with the BPSL0636 mutant showed the highest rate of survival out of all strains tested and one mouse with sterile immunity, one surviving mouse did show persistence of the vaccine strain after 123 days. Mice vaccinated with the BPSS0015 and BPSS1818 mutant strains cleared the vaccination strain but did have residual wild-type Bp 1026b infection in the lungs, livers, and spleens.

Virulence Factor Double Mutants are Cleared and Give Partial Protection Against Lethal Bp Challenge. Double mutants of these virulence factors were generated in a Bp 1026b background, using X-Red recombineering to determine if they could be completely cleared from the host and maintain protection as potential live-attenuated vaccines against lethal wild-type pp infection. Mutants of all combinations of these virulence factors were made to fully evaluate the protective effect the mutants have on BALB/c mice. Three double mutant strains, BPSL0636/BPSS0015, BPSL0636/BPSS1818, and BPSS0015/BPSS1818, were used to vaccinate (i.n.) groups of five BALB/c mice 60 days prior to wild-type Bp challenge. Vaccinated mice were challenged (i.n.) with 4,500 CFU of wild-type Bp 1026b and monitored for 63 days. Control mice vaccinated with PBS showed signs of murine melioidosis and 100% mortality within the first week of infection, while 20-60% of mutant vaccinated mice were able to survive until the study was terminated. All double mutant strains were completely cleared from surviving mice indicating that the combined mutations reduced the persistence of these strains in the host. Mice vaccinated with the BPSL0636/BPSS0015 mutant showed the highest survival at 60% and one mouse with an extended survival to 22 days post-infection. Of the surviving mice vaccinated with the BPSL0636/BPSS0015 mutant, one showed sterile immunity while the others showed infection in the lungs, liver, and spleen. Mice vaccinated with the BPSL0636/BPSS1818 mutant showed 20% survival and the surviving mouse had 2×10⁷ CFU wild-type Bp 1026b in the spleen but had cleared the infection from the lungs and liver. Mice vaccinated with the BPSS0015/BPSS1818 mutant showed 40% survival (2 out of 5 survived), one surviving mouse with sterile immunity, and another with a persistent infection of wild-type Bp in the lungs, liver and spleen. The BPSS0015/BPSS1818 mutant strain also extended the survival of another mouse to 38 days post-infection —. Creating mutants that were defective in two of the virulence factors allowed complete clearance of the mutant strains from the host, while maintaining various levels of protection against lethal Bp infection.

To further test these mutant strains as potential live-attenuated vaccines, the window between vaccination and challenge was shortened by 30 days to determine if this would impact their protective capabilities. As described above, groups of five BALB/c mice were vaccinated (i.n.) with BPSL0636/BPSS0015, BPSL0636/BPSS1818, or BPSS0015/BPSS18 double mutants and then challenged (i.n.) with wild-type Bp 30 days post-vaccination. As expected, BALB/c mice vaccinated with only PBS showed 100% mortality within the first 6 days post-infection. Interestingly, mice vaccinated with the BPSL0636/BPSS1818 mutant or the BPSS0015/BPSS1818 mutant also showed 100% mortality within the first week post-infection. However, BALB/c mice vaccinated with the BPSL0636/BPSS0015 double mutant showed 40% survival for the duration of the study and a mouse with an extended therapeutic window to day 62 post-infection. Both of the surviving mice cleared the BPSL0636/BPSS0015 double mutant. One surviving mouse showed complete clearance of wild-type Bp 1026b, while the other had infection in the lungs, liver and spleen. These data indicate that the BPSL0636/BPSS1818 and BPSS0015/BPSS1818 mutant strains are not as effective as live attenuated vaccines when challenged 30 days post-vaccination while the BPSL0636/BPSS0015 mutant was still able to provide protection.

The BPSL0636/BPSS0015 Mutant Protects BALB/c and C57BL/6 Mice Against Lethal Bp Challenge. To improve the BPSL0636/BPSS0015 mutant as a potential vaccine, a homologous booster immunization was added. Groups of five BALB/c mice were vaccinated (i.n.) with the BPSL0636/BPSS0015 mutant strain or PBS control and given an equivalent immunization 30 days later. Thirty-three days after the booster immunization, mice were challenged with a lethal dose of wild-type Bp 1026b (4,500 CPU) and monitored for 62 days. As expected, BALB/c mice vaccinated with PBS showed rapid disease progression with an average weight loss of 17% and worsening clinical symptoms post-challenge leading to 100% mortality two days post-challenge. In contrast, the BPSL0636/BPSS0015 mutant vaccinated mice lost an average of 7% body weight and only showed minor clinical symptoms post-challenge. Shortly after challenge, mice vaccinated with the BPSL0636/BPSS0015 mutant were able to recover lost weight and showed reduced clinical symptoms. One hundred percent of the BALB/c mice that were vaccinated and given a booster of the BPSL0636/BPSS0015 mutant strain were able to survive the entire length of the study (60 days) against a lethal Bp challenge. All surviving mice showed some level of persistence of wild-type Bp 1026b in the lungs, liver, and spleen but no detectable level of the BPSL0636/BPSS0015 mutant strain was present.

To validate the BPSL0636/BPSS0015 mutant strain as a live-attenuated vaccine, it was tested in another in vivo model. C57BL/6 mice have been widely used to study the pathogenesis of Bp, test attenuated vaccine strains, and are more resistant to Bp infection than BALB/c mice (Silva et al. (2013) Infect. Imrnun. 81:4626-4634; Khakhum et al. (2019) mSphere 4; Morici et al. (2019) Clin. Exp. Immunol. 196:178-188). Following the same schedule used for vaccine efficacy testing in BALB/c mice, C57BL/6 mice were vaccinated (i.n.) with the BPSL0636/BPSS0015 mutant strain and given a booster immunization 30 days later. BPSL0636/BPSS0015 mutant vaccinated mice and PBS vaccinated mice were challenged with a lethal dose of virulent Bp 1026b (12,000 CFU) and monitored for 62 days. As expected, C57BL/6 mice vaccinated with PBS showed an average of 14% body weight loss and rapidly increasing clinical symptoms within the first two days post-challenge. These factors led to 100% mortality of PBS vaccinated mice by day three post-challenge. All five C57BL/6 mice vaccinated with the BPSL0636/BPSS0015 mutant were protected from lethal challenge with virulent Bp for the duration of the study (60 days). Although all mice had some level of chronic wild-type Bp infection in the lungs, liver, and spleen, the BPSL0636/BPSS0015 mutant strain was cleared from the host, aligning with the results from the BALB/c challenge. No clinical symptoms were present in any of the BPSL0636/BPSS0015 mutant vaccinated C57BL/6 mice at the end of the study. Taken together, the protection provided by the BPSL0636/BPSS0015 mutant strain in two different mouse models extends the window of survival that could allow for antimicrobial treatment and supports the use of this mutant as a live-attenuated vaccine against melioidosis.

What is claimed is:

1. A composition comprising
(a) a recombinant Surface Attachment Protein 1 (Sap1) polypeptide having the amino acid sequence of SEQ ID NO:1,
(b) a recombinant Surface Attachment Protein 2 (Sap2) polypeptide having the amino acid sequence of SEQ ID NO:10, or
(c) a combination of (a) and (b),
wherein the recombinant Sap1 polypeptide and/or recombinant Sap2 polypeptide is tagged and/or in admixture with an adjuvant.

2. The composition of claim 1, wherein the Sap2 polypeptide further comprises a signal peptide.

3. A method of immunizing against a Burkholderia infection comprising administering to a subject in need thereof a composition of claim 1 thereby immunizing the subject against a Burkholderia infection.

4. A kit for diagnosing a Burkholderia infection comprising a recombinant Surface Attachment Protein immobilized on a substrate, the recombinant Surface Attachment Protein comprising:
(a) a recombinant Surface Attachment Protein 1 (Sap1) polypeptide having the amino acid sequence of SEQ ID NO:1,
(b) a recombinant Surface Attachment Protein 2 (Sap2) polypeptide having the amino acid sequence of SEQ ID NO:10, or
(c) a combination of (a) and (b).

5. A composition comprising a preservative; and
(a) an isolated Surface Attachment Protein 1 (Sap1) antibody having a variable domain comprising
(i) a CDR1 of SEQ ID NO:14,
(ii) a CDR2 of SEQ ID NO:18, and
(iii) a CDR3 of SEQ ID NO:22;
(b) an isolated Surface Attachment Protein 2 (Sap2) antibody comprising a variable domain comprising
(i) a CDR1 of SEQ ID NO:26,
(ii) a CDR2 of SEQ ID NO:29, and
(iii) a CDR3 of SEQ ID NO:32; or
(c) a combination of (a) and (b).

6. The composition of claim 5, wherein the variable domain of the Sap1 antibody comprises
(i) the CDR1 of SEQ ID NO:15, SEQ ID NO:16, or SEQ ID NO:17;
(ii) the CDR2 of SEQ ID NO:19, SEQ ID NO:20, or SEQ ID NO:21; and
(iii) the CDR3 of SEQ ID NO:23, SEQ ID NO:24, or SEQ ID NO:25.

7. The composition of claim 5, wherein the variable domain of the Sap2 antibody comprises
(i) the CORI of SEQ ID NO:27 or SEQ ID NO:28;
(ii) the CDR2 of SEQ ID NO:30 or SEQ ID NO:31; and
(iii) the CDR3 of SEQ ID NO:33 or SEQ ID NO:34.

8. A method of treating or attenuating a Burkholderia infection or Burkholderia-associated condition in a subject comprising administering to a subject in need of treatment an effective amount of a Surface Attachment Protein 1 (Sap1) antibody, a Surface Attachment Protein 2 (Sap2) antibody, or a combination thereof thereby treating or attenuating the Burkholderia infection or Burkholderia-associated condition in the subject.

9. The method of claim 8, wherein the antibody is a monoclonal antibody or nanobody.

10. The method of claim 8, wherein the Burkholderia-associated condition is melioidosis or pneumonia.

11. A live-attenuated Burkholderia pseudomallei comprising deletion of nucleic acids encoding the BPSL0636 polypeptide of SEQ ID NO:35.

12. The live-attenuated Burkholderia pseudomallei of claim 11, further comprising deletion of nucleic acids encoding the BPSS0015 polypeptide of SEQ ID NO:137.

13. A method of immunizing against a Burkholderia pseudomallei infection comprising administering to a subject in need thereof a live-attenuated Burkholderia pseudomallei of claim 11 thereby immunizing the subject against a Burkholderia pseudomallei infection.


SEQUENCE LISTING (137 sequences)

SEQ ID NO: 1 - protein, 92 aa, Artificial Sequence
  Synthetic polypeptide
  MISC_FEATURE (1)..(1): Xaa denotes Gly or Asn
  MISC_FEATURE (4)..(4): Xaa denotes Ser, Ala or Val
  MISC_FEATURE (5)..(5): Xaa denotes Ser or Ala
  MISC_FEATURE (6)..(6): Xaa denotes Val or Leu
  MISC_FEATURE (7)..(7): Xaa denotes Lys or Gln
  MISC_FEATURE (9)..(9): Xaa denotes Lys or Gln
  MISC_FEATURE (32)..(32): Xaa denotes Ser or Asn
  MISC_FEATURE (39)..(39): Xaa denotes Ser or Ala
  MISC_FEATURE (40)..(40): Xaa denotes Gln or Arg
  MISC_FEATURE (49)..(49): Xaa denotes Phe or Tyr
  MISC_FEATURE (64)..(64): Xaa denotes Ser or Leu
  MISC_FEATURE (66)..(66): Xaa denotes Glu or Asp
  MISC_FEATURE (67)..(67): Xaa denotes Gln, His or Lys
  MISC_FEATURE (76)..(76): Xaa denotes Glu or Asp
  MISC_FEATURE (87)..(87): Xaa denotes Ser or Ala
  MISC_FEATURE (90)..(90): Xaa denotes Ile or Val
XGDXXXXPXQEIQLTKNAWGCLSKDNLDSVLXHERDGKXXAKQQYFDDXRCLSVPEGQRFRVVXVXXGDVQFVSAXNSDQQGLWTDXRFXKQ

SEQ ID NO: 2 - protein, 115 aa, Burkholderia mallei
MLKRTGLFLALTGGIVAFSVAQANGDVSLKPQQEIQLTKNAWGCLSKDNLDSVLNHERDGKARAKQQYFDDYRCLSVPEGQRFRVVSVDKGDVQFVSAENSDQQGLWTDARFIKQ

SEQ ID NO: 3 - protein, 124 aa, Burkholderia pseudomallei
MTRANGRHDMLKRTGLFLALTGGIVAFSVAQANGDASLKPQQEIQLTKNAWGCLSKDNLDSVLNHERDGKAQAKQQYFDDYRCLSVPEGQRFRVVSVDKGDVQFVSAENSDQQGLWTDARFIKQ

SEQ ID NO: 4 - protein, 114 aa, Burkholderia pseudomallei
MKRTGLFLALTGGIVAFSVAQANGDASLKPQQEIQLTKNAWGCLSKDNLDSVLNHERDGKAQAKQQYFDDYRCLSVPEGQRFRVVSVDKGDVQFVSAENSDQQGLWTDARFIKQ

SEQ ID NO: 5 - protein, 115 aa, Burkholderia multivorans
MMKRTGVFLALVGACAMVSIAQAGGDVAVQPKQEIQLTKNAWGCLSKDNLDSVLSHERDGKAQAKQQYFDDFRCLSVPEGQRFRVVSVEHGDVQFVSADNSDQQGLWTDARFIKQ

SEQ ID NO: 6 - protein, 115 aa, Burkholderia cenocepacia
MMKRTGVLFALVGAFCAVSIAQAGGDSAVKPKQEIQLTKNAWGCLSKDNLDSVLSHERDGKSQAKQQYFDDFRCLSVPEGQRFRVVLVDQGDVQFVSADNSDQQGLWTDSRFVKQ

SEQ ID NO: 7 - protein, 114 aa, Burkholderia cenocepacia
MKRTGVLFALVGAFCAVSIAQAGGDSAVKPKQEIQLTKNAWGCLSKDNLDSVLSHERDGKSQAKQQYFDDFRCLSVPEGQRFRVVSVDQGDVQFVSADNSDQQGLWTDSRFVKQ

SEQ ID NO: 8 - protein, 115 aa, Burkholderia cenocepacia
MMKRTGVLFALVGAFCAVSIAQAGGDSAVKPKQEIQLTKNAWGCLSKDNLDSVLSHERDGKSQAKQQYFDDFRCLSVPEGQRFRVVSVDQGDVQFVSADNSDQQGLWTDSRFVKQ

SEQ ID NO: 9 - protein, 115 aa, Burkholderia cepacia
MMKRTGVFLALVGAFAAVSIAYAGGDSAVKPKQEIQLTKNAWGCLSKDNLDSVLSHERDGKAQAKQQYFDDYRCLSVPEGQRFRVVSVEQGDVQFVSADNSDQQGLWTDSRFIKQ

SEQ ID NO: 10 - protein, 375 aa, Artificial Sequence
  Synthetic polypeptide
  MISC_FEATURE (50)..(50): Xaa denotes Lys or Gln
  MISC_FEATURE (182)..(182): Xaa denotes Asn or Asp
  MISC_FEATURE (318)..(318): Xaa denotes Ala or Thr
AGACLNGSTIASTTRAPLVARQGSVFSSTLYDPAITSNNRTHNPVMLTVXVTNNGRPVAGCDVAWQPRGAGGASGWLFPASASTDANGIASAWWVAGSGAAQTAVASIRRFDGTTQGVAIGGSAQPHATRANSIHLNYEPASDWTAFRVDVTPEALAPTTYWEAIGWPGAYTGIQSIDGKQXGLVLFSVWDVNGKSPQIIAKGPGVDCTQFGGEGTGYKCAKRHAPVAGRTYRFMASIAPVAGQNQTDYSVWFTDTSTNARELIATLRYQKAVQSANYANSFVEDWATQGASCLGATQRAGQYGNVWALDRASAQWRXVKRASTSAVYTPDHNEVCSNYQFSVVNGRFRMSTGGHAVGQPLNLPNGPKSFPLTLP

SEQ ID NO: 11 - protein, 400 aa, Burkholderia pseudomallei
MRRIELKFNCIAATILAAVAADATAAGACLNGSTIASTTRAPLVARQGSVFSSTLYDPAITSNNRTHNPVMLTVQVTNNGRPVAGCDVAWQPRGAGGASGWLFPASASTDANGIASAWWVAGSGAAQTAVASIRRFDGTTQGVAIGGSAQPHATRANSIHLNYEPASDWTAFRVDVTPEALAPTTYWEAIGWPGAYTGIQSIDGKQNGLVLFSVWDVNGKSPQIIAKGPGVDCTQFGGEGTGYKCAKRHAPVAGRTYRFMASIAPVAGQNQTDYSVWFTDTSTNARELIATLRYQKAVQSANYANSFVEDWATQGASCLGATQRAGQYGNVWALDRASAQWRAVKRASTSAVYTPDHNEVCSNYQFSVVNGRFRMSTGGHAVGQPLNLPNGPKSFPLTLP

SEQ ID NO: 12 - protein, 395 aa, Burkholderia pseudomallei
MKFNCIAATILAAVAADATAAGACLNGSTIASTTRAPLVARQGSVFSSTLYDPAITSNNRTHNPVMLTVKVTNNGRPVAGCDVAWQPRGAGGASGWLFPASASTDANGIASAWWVAGSGAAQTAVASIRRFDGTTQGVAIGGSAQPHATRANSIHLNYEPASDWTAFRVDVTPEALAPTTYWEAIGWPGAYTGIQSIDGKQDGLVLFSVWDVNGKSPQIIAKGPGVDCTQFGGEGTGYKCAKRHAPVAGRTYRFMASIAPVAGQNQTDYSVWFTDTSTNARELIATLRYQKAVQSANYANSFVEDWATQGASCLGATQRAGQYGNVWALDRASAQWRTVKRASTSAVYTPDHNEVCSNYQFSVVNGRFRMSTGGHAVGQPLNLPNGPKSFPLTLP

SEQ ID NO: 13 - protein, 400 aa, Burkholderia mallei
MRRSELKFNCIAATILAAVAADATAAGACLNGSTIASTTRAPLVARQGSVFSSTLYDPAITSNNRTHNPVMLTVKVTNNGRPVAGCDVAWQPRGAGGASGWLFPASASTDANGIASAWWVAGSGAAQTAVASIRRFDGTTQGVAIGGSAQPHATRANSIHLNYEPASDWTAFRVDVTPEALAPTTYWEAIGWPGAYTGIQSIDGKQNGLVLFSVWDVNGKSPQIIAKGPGVDCTQFGGEGTGYKCAKRHAPVAGRTYRFMASIAPVAGQNQTDYSVWFTDTSTNARELIATLRYQKAVQSANYANSFVEDWATQGASCLGATQRAGQYGNVWALDRASAQWRTVKRASTSAVYTPDHNEVCSNYQFSVVNGRFRMSTGGHAVGQPLNLPNGPKSFPLTLP

SEQ ID NO: 14 - protein, 10 aa, Artificial Sequence
  Synthetic peptide
  MISC_FEATURE (1)..(1): Xaa denotes Gly or Arg
  MISC_FEATURE (4)..(4): Xaa denotes Ile or Phe
  MISC_FEATURE (6)..(6): Xaa denotes Ser or Arg
  MISC_FEATURE (9)..(9): Xaa denotes Met or Leu
XRTXSXYAXG

SEQ ID NO: 15 - protein, 10 aa, Artificial Sequence
  Synthetic peptide
GRTISSYAMG

SEQ ID NO: 16 - protein, 10 aa, Artificial Sequence
  Synthetic peptide
GRTFSRYAMG

SEQ ID NO: 17 - protein, 10 aa, Artificial Sequence
  Synthetic peptide
RRTFSSYALG

SEQ ID NO: 18 - protein, 17 aa, Artificial Sequence
  Synthetic peptide
  MISC_FEATURE (3)..(3): Xaa denotes Asn or Ser
  MISC_FEATURE (4)..(4): Xaa denotes Leu, Thr or Trp
  MISC_FEATURE (5)..(7): Xaa denotes Ser or Gly
  MISC_FEATURE (8)..(8): Xaa denotes Ser, Asn or Thr
  MISC_FEATURE (10)..(10): Xaa denotes Ser, Gly or His
AIXXXXXXTXYADSVSG

SEQ ID NO: 19 - protein, 17 aa, Artificial Sequence
  Synthetic peptide
AINLSSSSTSYADSVKG

SEQ ID NO: 20 - protein, 17 aa, Artificial Sequence
  Synthetic peptide
AISTSGGNTGYADSVKG

SEQ ID NO: 21 - protein, 17 aa, Artificial Sequence
  Synthetic peptide
AISWGGSTTHYADSVQG

SEQ ID NO: 22 - protein, 6 aa, Artificial Sequence
  Synthetic peptide
  MISC_FEATURE (1)..(1): Xaa denotes Ala, Ser or Thr
  MISC_FEATURE (2)..(2): Xaa denotes Asp or Asn
  MISC_FEATURE (3)..(3): Xaa denotes Gly or Phe
  MISC_FEATURE (4)..(4): Xaa denotes Ser or Ala
  MISC_FEATURE (5)..(5): Xaa denotes Asn, Ser or Tyr
XXXXXW

SEQ ID NO: 23 - protein, 6 aa, Artificial Sequence
  Synthetic peptide
ADGSNW

SEQ ID NO: 24 - protein, 15 aa, Artificial Sequence
  Synthetic peptide
RRSVGSMSASNFASW

SEQ ID NO: 25 - protein, 6 aa, Artificial Sequence
  Synthetic peptide
TNGAYW

SEQ ID NO: 26 - protein, 10 aa, Artificial Sequence
  Synthetic peptide
  MISC_FEATURE (3)..(3): Xaa denotes Arg or Ile
GSXFRFNAMG

SEQ ID NO: 27 - protein, 10 aa, Artificial Sequence
  Synthetic peptide
GSRFRFNAMG

SEQ ID NO: 28 - protein, 10 aa, Artificial Sequence
  Synthetic peptide
GSIFRFNAMG

SEQ ID NO: 29 - protein, 16 aa, Artificial Sequence
  Synthetic peptide
  MISC_FEATURE (1)..(1): Xaa denotes Ser or Asp
  MISC_FEATURE (3)..(3): Xaa denotes Thr or Ala
  MISC_FEATURE (6)..(6): Xaa denotes Asp ro Gly
  MISC_FEATURE (9)..(9): Xaa denotes Val or Asn
XIXSGXSTXYADSVKG

SEQ ID NO: 30 - protein, 16 aa, Artificial Sequence
  Synthetic peptide
SITSGDSTVYADSVKG

SEQ ID NO: 31 - protein, 16 aa, Artificial Sequence
  Synthetic peptide
DIASGGSTNYADSVKG

SEQ ID NO: 32 - protein, 15 aa, Artificial Sequence
  Synthetic peptide
  MISC_FEATURE (4)..(4): Xaa denotes His or Tyr
  MISC_FEATURE (6)..(6): Xaa denotes Arg or Tyr
  MISC_FEATURE (9)..(9): Xaa denotes Ala or Val
  MISC_FEATURE (10)..(10): Xaa denotes Phe or Tyr
  MISC_FEATURE (13)..(13): Xaa denotes Asp or Glu
QGSXRXPTXXEYXYW

SEQ ID NO: 33 - protein, 15 aa, Artificial Sequence
  Synthetic peptide
QGSHRYPTVFEYDYW

SEQ ID NO: 34 - protein, 15 aa, Artificial Sequence
  Synthetic peptide
QGSYRRPTAYEYEYW

SEQ ID NO: 35 - protein, 137 aa, Burkholderia pseudomallei
MSGQAAAAPVPSGIVVHAVSRVLELQYPSGECFRVPFELMRVYSPSAEVRGHGPGQETLQTGKREVTVTALEPVGNYALKPTFSDGHSTGIYSWALLYELATRQDALWREYFDKLKAAGVERDAPMPADSLPRGHHH

SEQ ID NO: 36 - DNA, 16 bp, Artificial Sequence
  Synthetic oligonucleotide
gcaacgcgatttctac

SEQ ID NO: 37 - DNA, 20 bp, Artificial Sequence
  Synthetic oligonucleotide
aattccacgcatatcaaggg

SEQ ID NO: 38 - DNA, 16 bp, Artificial Sequence
  Synthetic oligonucleotide
aaggcgaaccgataat

SEQ ID NO: 39 - DNA, 16 bp, Artificial Sequence
  Synthetic oligonucleotide
actgttctgggagcag

SEQ ID NO: 40 - DNA, 16 bp, Artificial Sequence
  Synthetic oligonucleotide
agagatcggcgagaag

SEQ ID NO: 41 - DNA, 17 bp, Artificial Sequence
  Synthetic oligonucleotide
aaagtgctcaatcaacg

SEQ ID NO: 42 - DNA, 20 bp, Artificial Sequence
  Synthetic oligonucleotide
aaacgactatcgttatgcgg

SEQ ID NO: 43 - DNA, 17 bp, Artificial Sequence
  Synthetic oligonucleotide
gaagaacgtgctgtaac

SEQ ID NO: 44 - DNA, 15 bp, Artificial Sequence
  Synthetic oligonucleotide
cggcgaatttcgacg

SEQ ID NO: 45 - DNA, 16 bp, Artificial Sequence
  Synthetic oligonucleotide
tgatgttcgcacgctc

SEQ ID NO: 46 - DNA, 16 bp, Artificial Sequence
  Synthetic oligonucleotide
ctcgagcaactgatcc

SEQ ID NO: 47 - DNA, 20 bp, Artificial Sequence
  Synthetic oligonucleotide
ctctacggcttcgacgaatg

SEQ ID NO: 48 - DNA, 16 bp, Artificial Sequence
  Synthetic oligonucleotide
gtgatcgccgagatag

SEQ ID NO: 49 - DNA, 16 bp, Artificial Sequence
  Synthetic oligonucleotide
tgacgatcgaaggcag

SEQ ID NO: 50 - DNA, 15 bp, Artificial Sequence
  Synthetic oligonucleotide
gcgacgaacgcatcc

SEQ ID NO: 51 - DNA, 18 bp, Artificial Sequence
  Synthetic oligonucleotide
gaatacgcgatcaatctg

SEQ ID NO: 52 - DNA, 20 bp, Artificial Sequence
  Synthetic oligonucleotide
actacgtgctcgaacagttc

SEQ ID NO: 53 - DNA, 16 bp, Artificial Sequence
  Synthetic oligonucleotide
tcggtgatctcgaaac

SEQ ID NO: 54 - DNA, 16 bp, Artificial Sequence
  Synthetic oligonucleotide
acatcgaccgctgggt

SEQ ID NO: 55 - DNA, 15 bp, Artificial Sequence
  Synthetic oligonucleotide
gccaggttcgcgatc

SEQ ID NO: 56 - DNA, 16 bp, Artificial Sequence
  Synthetic oligonucleotide
cgtgctgctctatttg

SEQ ID NO: 57 - DNA, 20 bp, Artificial Sequence
  Synthetic oligonucleotide
cgacgagcaggatgatgaac

SEQ ID NO: 58 - DNA, 16 bp, Artificial Sequence
  Synthetic oligonucleotide
gcgtaaacggaatcac

SEQ ID NO: 59 - DNA, 16 bp, Artificial Sequence
  Synthetic oligonucleotide
gatcatggtcggcatc

SEQ ID NO: 60 - DNA, 15 bp, Artificial Sequence
  Synthetic oligonucleotide
gtgccgatcgacgag

SEQ ID NO: 61 - DNA, 16 bp, Artificial Sequence
  Synthetic oligonucleotide
tcaggtcaagcacatt

SEQ ID NO: 62 - DNA, 20 bp, Artificial Sequence
  Synthetic oligonucleotide
cacgacctactccatcctaa

SEQ ID NO: 63 - DNA, 16 bp, Artificial Sequence
  Synthetic oligonucleotide
ccggacaggttgttat

SEQ ID NO: 64 - DNA, 16 bp, Artificial Sequence
  Synthetic oligonucleotide
gcagcaaaccatcctc

SEQ ID NO: 65 - DNA, 16 bp, Artificial Sequence
  Synthetic oligonucleotide
ccaagttcggtcgcag

SEQ ID NO: 66 - DNA, 40 bp, Artificial Sequence
  Synthetic oligonucleotide
ggatgcccagcagagctttgcgaatgggagacacgacatg

SEQ ID NO: 67 - DNA, 40 bp, Artificial Sequence
  Synthetic oligonucleotide
gcttgtacatcaggatgtggcgttactgcttgatgaagcg

SEQ ID NO: 68 - DNA, 40 bp, Artificial Sequence
  Synthetic oligonucleotide
ggatgcccagcagagctttgtgcacgaggaaatcgagcag

SEQ ID NO: 69 - DNA, 40 bp, Artificial Sequence
  Synthetic oligonucleotide
gcttgtacatcaggatgtggtcgttcagtggtggtggcct

SEQ ID NO: 70 - DNA, 40 bp, Artificial Sequence
  Synthetic oligonucleotide
ggatgcccagcagagctttgaacgaacgtcacccacggtc

SEQ ID NO: 71 - DNA, 40 bp, Artificial Sequence
  Synthetic oligonucleotide
gcttgtacatcaggatgtggcgcttcaacgcttcgcgaac

SEQ ID NO: 72 - DNA, 40 bp, Artificial Sequence
  Synthetic oligonucleotide
ggatgcccagcagagctttgttcccatcacatcgatggcg

SEQ ID NO: 73 - DNA, 40 bp, Artificial Sequence
  Synthetic oligonucleotide
gcttgtacatcaggatgtggtatctcacgcgccgttaacg

SEQ ID NO: 74 - DNA, 40 bp, Artificial Sequence
  Synthetic oligonucleotide
ggatgcccagcagagctttgtcggctaactcaatgggcag

SEQ ID NO: 75 - DNA, 40 bp, Artificial Sequence
  Synthetic oligonucleotide
gcttgtacatcaggatgtggtcacgacgagcagctcacct

SEQ ID NO: 76 - DNA, 40 bp, Artificial Sequence
  Synthetic oligonucleotide
ggatgcccagcagagctttgcaaacgcctaacgttctccg

SEQ ID NO: 77 - DNA, 40 bp, Artificial Sequence
  Synthetic oligonucleotide
gcttgtacatcaggatgtgggtcaggacagcacgagcttc

SEQ ID NO: 78 - DNA, 40 bp, Artificial Sequence
  Synthetic oligonucleotide
ggatgcccagcagagctttgctttgccgtttcccgtttcg

SEQ ID NO: 79 - DNA, 40 bp, Artificial Sequence
  Synthetic oligonucleotide
gcttgtacatcaggatgtgggccgtgctcaaagacgttgc

SEQ ID NO: 80 - DNA, 40 bp, Artificial Sequence
  Synthetic oligonucleotide
ggatgcccagcagagctttggacggtgagcgccgtggatg

SEQ ID NO: 81 - DNA, 40 bp, Artificial Sequence
  Synthetic oligonucleotide
gcttgtacatcaggatgtggtcgccgcgctagcgcgatct

SEQ ID NO: 82 - DNA, 40 bp, Artificial Sequence
  Synthetic oligonucleotide
ggatgcccagcagagctttgccgagacccacgcatgcaac

SEQ ID NO: 83 - DNA, 40 bp, Artificial Sequence
  Synthetic oligonucleotide
gcttgtacatcaggatgtgggacattcaacggccggatgc

SEQ ID NO: 84 - DNA, 40 bp, Artificial Sequence
  Synthetic oligonucleotide
ggatgcccagcagagctttgcgtttcggcgagtgaccatg

SEQ ID NO: 85 - DNA, 40 bp, Artificial Sequence
  Synthetic oligonucleotide
gcttgtacatcaggatgtggattcgcgctcagctcagcag

SEQ ID NO: 86 - DNA, 40 bp, Artificial Sequence
  Synthetic oligonucleotide
ggatgcccagcagagctttggcgacgtatacccgcgaatc

SEQ ID NO: 87 - DNA, 40 bp, Artificial Sequence
  Synthetic oligonucleotide
gcttgtacatcaggatgtggacgcatcagggcagcgtcag

SEQ ID NO: 88 - DNA, 65 bp, Artificial Sequence
  Synthetic oligonucleotide
atcccctgattccctttgtcaacagcaatggatatcagccgttgaaattcgccgctattgcggtc

SEQ ID NO: 89 - DNA, 101 bp, Artificial Sequence
  Synthetic oligonucleotide
gatgtgcttaaaaacttactcaatggaattagctcgagttaggtgtcacgcgtagtccgggacgtcgtacgggtagccgccctgcttgatgaagcgcgcgt

SEQ ID NO: 90 - DNA, 66 bp, Artificial Sequence
  Synthetic oligonucleotide
atcccctgattccctttgtcaacagcaatggatatcagccgttgattattcgatcgaatccgcttc

SEQ ID NO: 91 - DNA, 101 bp, Artificial Sequence
  Synthetic oligonucleotide
gatgtgcttaaaaacttactcaatggaattagctcgagttaggtgtcacgcgtagtccgggacgtcgtacgggtagccgccgggcagcgtcagcggaaacg

SEQ ID NO: 92 - DNA, 279 bp, Artificial Sequence
  Synthetic polynucleotide
atgaacggcgatgcgagcctgaaaccgcagcaagaaattcaactgaccaaaaatgcctggggctgcctgagcaaagataacctggactctgtgctgaatcatgaacgtgatggtaaagcacaggctaaacagcaatattttgatgactaccgctgtctgagcgttccggaaggccagcgttttcgcgttgttagtgtcgataaaggtgacgtccaattcgtgtccgcggaaaactcggatcaacaaggtctgtggacggacgcccgcttcatcaaacaa

SEQ ID NO: 93 - protein, 22 aa, Artificial Sequence
  Synthetic peptide
MKRTGLFLALTGGIVAFSVAQA

SEQ ID NO: 94 - protein, 96 aa, Artificial Sequence
  Synthetic polypeptide
MNGDASLKPQQEIQLTKNAWGCLSKDNLDSVLNHERDGKAQAKQQYFDDYRCLSVPEGQRFRVVSVDKGDVQFVSAENSDQQGLWTDARFIKQAAA

SEQ ID NO: 95 - DNA, 6746 bp, Artificial Sequence
  Synthetic polynucleotide
aactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagctatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagccgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcccggatctcgacgctctcccttatgcgactcctgcattaggaagcagcccagtagtaggttgaggccgttgagcaccgccgccgcaaggaatggtgcatgccgccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcggaattaattcccggtttaaaccggggatctcgatcccgcgaaattaatacgactcactataggggaattgtgagcggataacaattcccctctagaaataattttgtttaactttaagaaggagatatacatatgaacggcgatgcgagcctgaaaccgcagcaagaaattcaactgaccaaaaatgcctggggctgcctgagcaaagataacctggactctgtgctgaatcatgaacgtgatggtaaagcacaggctaaacagcaatattttgatgactaccgctgtctgagcgttccggaaggccagcgttttcgcgttgttagtgtcgataaaggtgacgtccaattcgtgtccgcggaaaactcggatcaacaaggtctgtggacggacgcccgcttcatcaaacaagccgccgcctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatgactgcaggaaggggatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 96 - protein, 20 aa, Artificial Sequence
  Synthetic peptide
MKFNCIAATILAAVAADATA

SEQ ID NO: 97 - DNA, 1245 bp, Artificial Sequence
  Synthetic polynucleotide
catggcgaatcgaaatgcccgatgaggaggagcgaattgaaattcaactgcatcgcagcaaccatcctggccgcggtcgccgccgacgcgacggcggccggcgcgtgcctgaacggctccacgatcgcctcgacgacgcgtgcgccgctcgtcgcgcgacagggctcggtgttctcgtcgacgctctatgatccggcgatcacgtcgaacaatcgcacgcacaatcccgtcatgctgacggtcaaggtgacgaacaacgggcggccggtcgccggctgcgacgtcgcctggcagccgcgcggcgcgggcggcgcgtcgggatggctcttcccggcgagcgcgtcgaccgacgccaacgggatcgcgtccgcgtggtgggtcgccggcagcggcgccgcgcagacggcggtggcgagcatccgccggttcgacggcacgacgcagggcgtcgcgatcggcggctcggcgcagccgcacgcgacgcgcgcgaattcgatccatctgaactacgagccggcgagcgactggaccgcgttcagggtcgacgtgacgcccgaagcgctcgcgccgaccacctattgggaggcgatcggctggccgggcgcgtacaccggcatccagtcgatcgacggcaagcaggatggactcgtgctgttctccgtgtgggacgtgaacggcaagtcgcctcaaatcatcgcgaaagggcccggcgtcgattgcacgcagttcggcggcgaaggcaccggctacaagtgcgccaagcgccatgcgcccgtcgcgggccgcacctaccgcttcatggcgtcgatcgcgccggtcgcaggacagaaccagaccgactattcggtctggttcaccgacacctcgaccaacgcgcgcgagctgatcgcgacgctgcgctaccagaaggccgtccagtcggcgaactacgcgaacagcttcgtcgaggattgggcgacgcagggcgcgtcgtgtctcggcgcgacgcagcgcgccgggcagtacggcaacgtatgggcgctcgacagggcgtccgcgcagtggcgcacggtcaagcgggcatcgacctcggccgtctacacgccggatcacaacgaagtctgctcgaactatcagttctcggtggtcaacggccgcttccggatgagcacgggcggccatgcggtcggacagccgctcaatctgccgaacgggccgaagtcgtttccgctgacgctgcccggcggccaccaccaccaccaccac

SEQ ID NO: 98 - protein, 403 aa, Artificial Sequence
  Synthetic polypeptide
MKFNCIAATILAAVAADATAAGACLNGSTIASTTRAPLVARQGSVFSSTLYDPAITSNNRTHNPVMLTVKVTNNGRPVAGCDVAWQPRGAGGASGWLFPASASTDANGIASAWWVAGSGAAQTAVASIRRFDGTTQGVAIGGSAQPHATRANSIHLNYEPASDWTAFRVDVTPEALAPTTYWEAIGWPGAYTGIQSIDGKQDGLVLFSVWDVNGKSPQIIAKGPGVDCTQFGGEGTGYKCAKRHAPVAGRTYRFMASIAPVAGQNQTDYSVWFTDTSTNARELIATLRYQKAVQSANYANSFVEDWATQGASCLGATQRAGQYGNVWALDRASAQWRTVKRASTSAVYTPDHNEVCSNYQFSVVNGRFRMSTGGHAVGQPLNLPNGPKSFPLTLPGGHHHHHH

SEQ ID NO: 99 - DNA, 8005 bp, Artificial Sequence
  Synthetic polynucleotide
tcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagtcgaccaataactctacggggtctgacgctcagtggaacgaaatcgatgagctcgcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctagcctcgggcatccaagctagctaagcgcgttacgccgtgggtcgatgtttgatgttatggaacagcaacgatgttacgcagcagggtagtcgccctaaaacaaagttaggcagccgttgtgctggtgctttctagtagttgttgtggggtaggcagtcagagctcgatttgcttgtcgccataatagattcacaagaaggattcgacatgggtcaaagtagcgatgaagccaacgctcccgttgcagggcagtttgcgcttcccctgagtgccacctttggcttaggggatcgcgtacgcaagaaatctggtgccgcttggcagggtcaagtcgtcggttggtattgcacaaaactcactcctgaaggctatgcggtcgagtccgaatcccacccaggctcagtgcaaatttatcctgtggctgcacttgaacgtgtggcctaagcgatatcttaggatctcccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggaggaattcaccatggcgaatcgaaatgcccgatgaggaggagcgaattgaaattcaactgcatcgcagcaaccatcctggccgcggtcgccgccgacgcgacggcggccggcgcgtgcctgaacggctccacgatcgcctcgacgacgcgtgcgccgctcgtcgcgcgacagggctcggtgttctcgtcgacgctctatgatccggcgatcacgtcgaacaatcgcacgcacaatcccgtcatgctgacggtcaaggtgacgaacaacgggcggccggtcgccggctgcgacgtcgcctggcagccgcgcggcgcgggcggcgcgtcgggatggctcttcccggcgagcgcgtcgaccgacgccaacgggatcgcgtccgcgtggtgggtcgccggcagcggcgccgcgcagacggcggtggcgagcatccgccggttcgacggcacgacgcagggcgtcgcgatcggcggctcggcgcagccgcacgcgacgcgcgcgaattcgatccatctgaactacgagccggcgagcgactggaccgcgttcagggtcgacgtgacgcccgaagcgctcgcgccgaccacctattgggaggcgatcggctggccgggcgcgtacaccggcatccagtcgatcgacggcaagcaggatggactcgtgctgttctccgtgtgggacgtgaacggcaagtcgcctcaaatcatcgcgaaagggcccggcgtcgattgcacgcagttcggcggcgaaggcaccggctacaagtgcgccaagcgccatgcgcccgtcgcgggccgcacctaccgcttcatggcgtcgatcgcgccggtcgcaggacagaaccagaccgactattcggtctggttcaccgacacctcgaccaacgcgcgcgagctgatcgcgacgctgcgctaccagaaggccgtccagtcggcgaactacgcgaacagcttcgtcgaggattgggcgacgcagggcgcgtcgtgtctcggcgcgacgcagcgcgccgggcagtacggcaacgtatgggcgctcgacagggcgtccgcgcagtggcgcacggtcaagcgggcatcgacctcggccgtctacacgccggatcacaacgaagtctgctcgaactatcagttctcggtggtcaacggccgcttccggatgagcacgggcggccatgcggtcggacagccgctcaatctgccgaacgggccgaagtcgtttccgctgacgctgcccggcggccaccaccaccaccaccactgatctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttccactcactacagcagagccatttaaacaacatcccctccccctttccaccgcgtcagacgcccgtagcagcccgctacgggctttttcatgccctgccctagcgtccaagcctcacggccgcgctcggcctctctggcggccttctggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagcgagaaatcaccatgagtgacgactgaatccggtgagaatggcaaaagctaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcctcgtgatacgcctatttttataggttaatg

SEQ ID NO: 100 - protein, 134 aa, Artificial Sequence
  Synthetic polypeptide
MKYLLPTAAAGLLLLAAQPAMAQVQLQESGGGLVQAGGSLRLSCEATGRTISSYAMGWFRQAPGKEREFVAAINLSSSSTSYADSVKGRFTISRDNAKNTVYLQMNSLKPEDTAVYTCADGSNWGQGTQVTVSS

SEQ ID NO: 101 - protein, 146 aa, Artificial Sequence
  Synthetic polypeptide
MKYLLPTAAAGLLLLAAQPAMAQVQLQESGGGLVQAGGSLRLSCAASGRTFSRYAMGWFRQAPGKEREFVAAISTSGGNTGYADSVKGRFTISRDNAKNTVYYLEMTSLKPEDTAVYYCNARRSVGSMSASNFASWGQGTQVTVSS

SEQ ID NO: 102 - protein, 135 aa, Artificial Sequence
  Synthetic polypeptide
MKYLLPTAAAGLLLLAAQPAMAQVQLQESGGGLVQAGGSLSLSCTASRRTFSSYALGWFRQAPGKEREFVAAISWGGSTTHYADSVQGRFAISRDNAKNTVYYLQMNNLKPEDTAVYYCTNGAYWGQGTQVTVSS

SEQ ID NO: 103 - protein, 146 aa, Artificial Sequence
  Synthetic polypeptide
MKYLLPTAAAGLLLLAAQPAMAQVQLQESGGGLVQAGGSLRLSCAASGSRFRFNAMGWYRQAPGKQRELVASITSGDSTVYADSVKGRFTISRHNANNTAYAYLQMNNLKAEDTAVYYCRHQGSHRYPTVFEYDYWGQGTQVTVSS

SEQ ID NO: 104 - protein, 125 aa, Artificial Sequence
  Synthetic polypeptide
MAQVQLQESGGGSVQAGGSLRLSCAASGSIFRFNAMGWYRQAPGKQREFVADIASGGSTNYADSVKGRFAISRDNAKNTVYYLQMNNLKAEDTAVYYCRHQGSYRRPTAYEYEYWGQGTQVTVSS

SEQ ID NO: 105 - DNA, 22 bp, Artificial Sequence
  Synthetic oligonucleotide
attagcttacgacgctacaccc

SEQ ID NO: 106 - DNA, 60 bp, Artificial Sequence
  Synthetic oligonucleotide
gatccacagcgcaacggcccggcatgcgccgggccgtgatcggttcagctggcacgacag

SEQ ID NO: 107 - DNA, 60 bp, Artificial Sequence
  Synthetic oligonucleotide
aaacacatagctaacccagcctggtgcccgcaagcctggagacagggcgattaagttggg

SEQ ID NO: 108 - DNA, 15 bp, Artificial Sequence
  Synthetic oligonucleotide
tgcgcgatccacagc

SEQ ID NO: 109 - DNA, 23 bp, Artificial Sequence
  Synthetic oligonucleotide
tcacctttaaacacatagctaac

SEQ ID NO: 110 - DNA, 17 bp, Artificial Sequence
  Synthetic oligonucleotide
gcattcggttcggtcgg

SEQ ID NO: 111 - DNA, 21 bp, Artificial Sequence
  Synthetic oligonucleotide
tgtgcttgctatcgtttaccg

SEQ ID NO: 112 - DNA, 21 bp, Artificial Sequence
  Synthetic oligonucleotide
gtgtgaaaccacttcgtcttg

SEQ ID NO: 113 - DNA, 22 bp, Artificial Sequence
  Synthetic oligonucleotide
gaagatcgtgctcggcgaaatg

SEQ ID NO: 114 - DNA, 22 bp, Artificial Sequence
  Synthetic oligonucleotide
ttcctgcgttcggtgccagtcg

SEQ ID NO: 115 - DNA, 20 bp, Artificial Sequence
  Synthetic oligonucleotide
ccgagctgctgaagaacacc

SEQ ID NO: 116 - DNA, 24 bp, Artificial Sequence
  Synthetic oligonucleotide
tatatggatccgcgggaatggacg

SEQ ID NO: 117 - DNA, 27 bp, Artificial Sequence
  Synthetic oligonucleotide
atatctgcagtggaatcgtttcggatg

SEQ ID NO: 118 - DNA, 33 bp, Artificial Sequence
  Synthetic oligonucleotide
atatctgcagcgataaattcatatcgtttcggc

SEQ ID NO: 119 - DNA, 28 bp, Artificial Sequence
  Synthetic oligonucleotide
tatatggatccgcgcgttactgcttgat

SEQ ID NO: 120 - DNA, 26 bp, Artificial Sequence
  Synthetic oligonucleotide
atataagcttggaatcgtttcggatg

SEQ ID NO: 121 - DNA, 25 bp, Artificial Sequence
  Synthetic oligonucleotide
tatatactagtcgcgggaatggacg

SEQ ID NO: 122 - DNA, 33 bp, Artificial Sequence
  Synthetic oligonucleotide
atataagcttcgataaattcatatcgtttcggc

SEQ ID NO: 123 - DNA, 29 bp, Artificial Sequence
  Synthetic oligonucleotide
tatatactagtcgcgcgttactgcttgat

SEQ ID NO: 124 - DNA, 24 bp, Artificial Sequence
  Synthetic oligonucleotide
tatatggtaccgcgggaatggacg

SEQ ID NO: 125 - DNA, 27 bp, Artificial Sequence
  Synthetic oligonucleotide
atatggtacctggaatcgtttcggatg

SEQ ID NO: 126 - DNA, 26 bp, Artificial Sequence
  Synthetic oligonucleotide
tatatggtaccgcgcgttactgcttg

SEQ ID NO: 127 - DNA, 32 bp, Artificial Sequence
  Synthetic oligonucleotide
atatggtaccgataaattcatatcgtttcggc

SEQ ID NO: 128 - DNA, 19 bp, Artificial Sequence
  Synthetic oligonucleotide
ggtaccgacgtcgacaagc

SEQ ID NO: 129 - DNA, 20 bp, Artificial Sequence
  Synthetic oligonucleotide
cgacaagccgaggaatctgg

SEQ ID NO: 130 - DNA, 19 bp, Artificial Sequence
  Synthetic oligonucleotide
gctgctcgcgtatcacacc

SEQ ID NO: 131 - protein, 8 aa, Artificial Sequence
  Synthetic peptide
WSHPQFEK

SEQ ID NO: 132 - protein, 10 aa, Artificial Sequence
  Synthetic peptide
EQKLISEEDL

SEQ ID NO: 133 - protein, 8 aa, Artificial Sequence
  Synthetic peptide
DYKDDDDK

SEQ ID NO: 134 - protein, 6 aa, Artificial Sequence
  Synthetic peptide
HHHHHH

SEQ ID NO: 135 - protein, 9 aa, Artificial Sequence
  Synthetic peptide
YPYDVPDYA

SEQ ID NO: 136 - protein, 6 aa, Artificial Sequence
  Synthetic peptide
CCPGCC

SEQ ID NO: 137 - protein, 154 aa, Burkholderia pseudomallei.
MPIFVVLFALAALIWGAIHMFEAIAARFGDAVAIGAATVAAVAIAAAIARSVRRRRDIAPNAREDGWTHVLQRAWGDLRISATKGLLWLSQDGADGRYTLTDLSGCRAETLDGRWYLVVAVRDDRRAEWKLPMDDKRDALRWARVLTLAKRQRL